SEQUENCE LISTING (102 sequences)

SEQ ID NO: 1 - protein, 213 aa, Artificial Sequence
  Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 224 aa, Artificial Sequence
  Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 3 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 213 aa, Artificial Sequence
  Synthetic polypeptide
QIQLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 213 aa, Artificial Sequence
  Synthetic polypeptide
QIVLSQSPSILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 213 aa, Artificial Sequence
  Synthetic polypeptide
QIVLSQSPASLSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 213 aa, Artificial Sequence
  Synthetic polypeptide
QIVLSQSPAILSASPGEKVTMTCRASSSVSYIHWFQQKPGSSPKPWIYATSNLASGVPSRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 213 aa, Artificial Sequence
  Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNADQSGNSQESVTEQDSKDSTYSLSSTLTLSKADYEKHKVYACEVTHQGLSSPVTKSFNRGEC

SEQ ID NO: 9 - protein, 224 aa, Artificial Sequence
  Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVSQSSGLYSLSSVVTVPSSSLGTQTYICNVNHKPSNTKVDKKVEPKSC

SEQ ID NO: 10 - protein, 213 aa, Artificial Sequence
  Synthetic polypeptide
QIVLSQSPSSLSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 213 aa, Artificial Sequence
  Synthetic polypeptide
QIQLSQSPSILSASPGEKVTMTCRASSSVSYIHWFQQKPGSSPKPWIYATSNLASGVPSRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 213 aa, Artificial Sequence
  Synthetic polypeptide
QIQLSQSPSSLSASPGEKVTMTCRASSSVSYIHWFQQKPGSSPKPWIYATSNLASGVPSRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 213 aa, Artificial Sequence
  Synthetic polypeptide
QIQLSQSPSILSASPGEKVTMTCRASSSVSYIHWFQQKPGSSPKPWIYATSNLASGVPSRFSGSGSGTSYSLTISRVEAEDAATYYCQQWTSNPPTFGGGTKLEIKRTVAAPSVFIFPPSDEQLKSGTASVVCLLNNFYPREAKVQWKVDNADQSGNSQESVTEQDSKDSTYSLSSTLTLSKADYEKHKVYACEVTHQGLSSPVTKSFNRGEC

SEQ ID NO: 14 - protein, 213 aa, Artificial Sequence
  Synthetic polypeptide
QIQLSQSPSSLSASPGEKVTMTCRASSSVSYIHWFQQKPGSSPKPWIYATSNLASGVPSRFSGSGSGTSYSLTISRVEAEDAATYYCQQWTSNPPTFGGGTKLEIKRTVAAPSVFIFPPSDEQLKSGTASVVCLLNNFYPREAKVQWKVDNADQSGNSQESVTEQDSKDSTYSLSSTLTLSKADYEKHKVYACEVTHQGLSSPVTKSFNRGEC

SEQ ID NO: 15 - protein, 227 aa, Artificial Sequence
  Synthetic polypeptide
DKTHTCPPCPAPELKGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 227 aa, Artificial Sequence
  Synthetic polypeptide
DKTHTCPPCPAPELLGGPSVFLFPPKPKDTLMKSRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGKEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVKHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 213 aa, Artificial Sequence
  Synthetic polypeptide
QIXLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 213 aa, Artificial Sequence
  Synthetic polypeptide
QIVLSQSPXILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 213 aa, Artificial Sequence
  Synthetic polypeptide
QIVLSQSPAXLSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 213 aa, Artificial Sequence
  Synthetic polypeptide
QIVLSQSPAILSASPGEKVTMTCRASSSVSYIHWFQQKPGSSPKPWIYATSNLASGVPXRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 213 aa, Artificial Sequence
  Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNAXQSGNSQESVTEQDSKDSTYSLSSTLTLSKADYEKHKVYACEVTHQGLSSPVTKSFNRGEC

SEQ ID NO: 24 - protein, 224 aa, Artificial Sequence
  Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVXQSSGLYSLSSVVTVPSSSLGTQTYICNVNHKPSNTKVDKKVEPKSC

SEQ ID NO: 25 - protein, 213 aa, Artificial Sequence
  Synthetic polypeptide
QIVLSQSPXXLSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 213 aa, Artificial Sequence
  Synthetic polypeptide
QIXLSQSPXILSASPGEKVTMTCRASSSVSYIHWFQQKPGSSPKPWIYATSNLASGVPXRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 213 aa, Artificial Sequence
  Synthetic polypeptide
QIXLSQSPXXLSASPGEKVTMTCRASSSVSYIHWFQQKPGSSPKPWIYATSNLASGVPXRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 213 aa, Artificial Sequence
  Synthetic polypeptide
QIXLSQSPXILSASPGEKVTMTCRASSSVSYIHWFQQKPGSSPKPWIYATSNLASGVPXRFSGSGSGTSYSLTISRVEAEDAATYYCQQWTSNPPTFGGGTKLEIKRTVAAPSVFIFPPSDEQLKSGTASVVCLLNNFYPREAKVQWKVDNAXQSGNSQESVTEQDSKDSTYSLSSTLTLSKADYEKHKVYACEVTHQGLSSPVTKSFNRGEC

SEQ ID NO: 29 - protein, 213 aa, Artificial Sequence
  Synthetic polypeptide
QIXLSQSPXXLSASPGEKVTMTCRASSSVSYIHWFQQKPGSSPKPWIYATSNLASGVPXRFSGSGSGTSYSLTISRVEAEDAATYYCQQWTSNPPTFGGGTKLEIKRTVAAPSVFIFPPSDEQLKSGTASVVCLLNNFYPREAKVQWKVDNAXQSGNSQESVTEQDSKDSTYSLSSTLTLSKADYEKHKVYACEVTHQGLSSPVTKSFNRGEC

SEQ ID NO: 30 - protein, 227 aa, Artificial Sequence
  Synthetic polypeptide
DKTHTCPPCPAPELXGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 227 aa, Artificial Sequence
  Synthetic polypeptide
DKTHTCPPCPAPELLGGPSVFLFPPKPKDTLMXSRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGXEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 227 aa, Artificial Sequence
  Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVXHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 214 aa, Artificial Sequence
  Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 221 aa, Artificial Sequence
  Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKS

SEQ ID NO: 36 - protein, 231 aa, Artificial Sequence
  Synthetic polypeptide
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 214 aa, Artificial Sequence
  Synthetic polypeptide
DIQLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 214 aa, Artificial Sequence
  Synthetic polypeptide
DILLTQSPSILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 214 aa, Artificial Sequence
  Synthetic polypeptide
DILLTQSPVSLSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 214 aa, Artificial Sequence
  Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNADQSGNSQESVTEQDSKDSTYSLSSTLTLSKADYEKHKVYACEVTHQGLSSPVTKSFNRGEC

SEQ ID NO: 41 - protein, 221 aa, Artificial Sequence
  Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVSQSSGLYSLSSVVTVPSSSLGTQTYICNVNHKPSNTKVDKRVEPKS

SEQ ID NO: 42 - protein, 214 aa, Artificial Sequence; identical to SEQ ID NO: 34
  Synthetic polypeptide

SEQ ID NO: 43 - protein, 214 aa, Artificial Sequence; identical to SEQ ID NO: 34
  Synthetic polypeptide

SEQ ID NO: 44 - protein, 214 aa, Artificial Sequence; identical to SEQ ID NO: 34
  Synthetic polypeptide

SEQ ID NO: 45 - protein, 214 aa, Artificial Sequence; identical to SEQ ID NO: 34
  Synthetic polypeptide

SEQ ID NO: 46 - protein, 214 aa, Artificial Sequence; identical to SEQ ID NO: 34
  Synthetic polypeptide

SEQ ID NO: 47 - protein, 231 aa, Artificial Sequence
  Synthetic polypeptide
PKSCDKTHTCPPCPAPELKGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 231 aa, Artificial Sequence
  Synthetic polypeptide
PKSCDKTHTCPPCPAPELLGGPSVFLFPPKPKDTLMKSRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 231 aa, Artificial Sequence
  Synthetic polypeptide
PKSCDKTHTCPPCPAPELLGGPSVFLFPPKPKDTLMISRTPEVTCVVVDVSHEDPEVKFNWYVDGKEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 231 aa, Artificial Sequence
  Synthetic polypeptide
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVKHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 214 aa, Artificial Sequence
  Synthetic polypeptide
DIXLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 214 aa, Artificial Sequence
  Synthetic polypeptide
DILLTQSPXILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 214 aa, Artificial Sequence
  Synthetic polypeptide
DILLTQSPVXLSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 214 aa, Artificial Sequence
  Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNAXQSGNSQESVTEQDSKDSTYSLSSTLTLSKADYEKHKVYACEVTHQGLSSPVTKSFNRGEC

SEQ ID NO: 55 - protein, 221 aa, Artificial Sequence
  Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVXQSSGLYSLSSVVTVPSSSLGTQTYICNVNHKPSNTKVDKRVEPKS

SEQ ID NO: 56 - protein, 214 aa, Artificial Sequence
  Synthetic polypeptide
DILLTQSPXXLSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 214 aa, Artificial Sequence
  Synthetic polypeptide
DIXLTQSPXILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 214 aa, Artificial Sequence
  Synthetic polypeptide
DIXLTQSPXXLSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 214 aa, Artificial Sequence
  Synthetic polypeptide
DIXLTQSPXILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNAXQSGNSQESVTEQDSKDSTYSLSSTLTLSKADYEKHKVYACEVTHQGLSSPVTKSFNRGEC

SEQ ID NO: 60 - protein, 214 aa, Artificial Sequence
  Synthetic polypeptide
DIXLTQSPXXLSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNAXQSGNSQESVTEQDSKDSTYSLSSTLTLSKADYEKHKVYACEVTHQGLSSPVTKSFNRGEC

SEQ ID NO: 61 - protein, 231 aa, Artificial Sequence
  Synthetic polypeptide
PKSCDKTHTCPPCPAPELXGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 231 aa, Artificial Sequence
  Synthetic polypeptide
PKSCDKTHTCPPCPAPELLGGPSVFLFPPKPKDTLMXSRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 231 aa, Artificial Sequence
  Synthetic polypeptide
PKSCDKTHTCPPCPAPELLGGPSVFLFPPKPKDTLMISRTPEVTCVVVDVSHEDPEVKFNWYVDGXEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 231 aa, Artificial Sequence
  Synthetic polypeptide
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVXHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 297 aa, Artificial Sequence
  Synthetic polypeptide
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 66 - protein, 1210 aa, Artificial Sequence [sequence omitted]

SEQ ID NO: 67 - protein, 722 aa, Artificial Sequence
  Synthetic polypeptide
GTCGACATGGATTTTCAGGTGCAGATTATCAGCTTCCTGCTAATCAGTGCTTCAGTCATAATGTCCAGAGGACAAATTGTTCTCTCCCAGTCTCCAGCAATCCTGTCTGCATCTCCAGGGGAGAAGGTCACAATGACTTGCAGGGCCAGCTCAAGTGTAAGTTACATCCACTGGTTCCAGCAGAAGCCAGGATCCTCCCCCAAACCCTGGATTTATGCCACATCCAACCTGGCTTCTGGAGTCCCTGTTCGCTTCAGTGGCAGTGGGTCTGGGACTTCTTACTCTCTCACAATCAGCAGAGTGGAGGCTGAAGATGCTGCCACTTATTACTGCCAGCAGTGGACTAGTAACCCACCCACGTTCGGAGGGGGGACCAAGCTGGAAATCAAACGTACGGTGGCTGCACCATCTGTCTTCATCTTCCCGCCATCTGATGAGCAGTTGAAATCTGGAACTGCCTCTGTTGTGTGCCTGCTGAATAACTTCTATCCCAGAGAGGCCAAAGTACAGTGGAAGGTGGATAACGCCCTCCAATCGGGTAACTCCCAGGAGAGTGTCACAGAGCAGGACAGCAAGGACAGCACCTACAGCCTCAGCAGCACCCTGACGCTGAGCAAAGCAGACTACGAGAAACACAAAGTCTACGCCTGCGAAGTCACCCATCAGGGCCTGAGCTCGCCCGTCACAAAGAGCTTCAACAGGGGAGAGTGTTGAGCGGCCGC

SEQ ID NO: 68 - DNA, 31 bp, Artificial Sequence
  Synthetic polynucleotide
ccagaggacaaattcaactctcccagtctcc

SEQ ID NO: 69 - DNA, 25 bp, Artificial Sequence
  Synthetic polynucleotide
cccagtctccatcaatcctgtctgc

SEQ ID NO: 70 - DNA, 25 bp, Artificial Sequence
  Synthetic polynucleotide
agtctccagcaagcctgtctgcatc

SEQ ID NO: 71 - DNA, 25 bp, Artificial Sequence
  Synthetic polynucleotide
ctggagtccctagtcgcttcagtgg

SEQ ID NO: 72 - DNA, 27 bp, Artificial Sequence
  Synthetic polynucleotide
gtggataacgccgaccaatcgggtaac

SEQ ID NO: 73 - DNA, 1427 bp, Artificial Sequence
  Synthetic polynucleotide
gtcgacatgggttggagcctcatcttgctcttccttgtcgctgttgctacgcgtgtcctgtcccaggtacaactgcagcagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgcagacaaatcctccagcacagcctacatgcagctcagcagcctgacatctgaggactctgcggtctattactgtgcaagatcgacttactacggcggtgactggtacttcaatgtctggggcgcagggaccacggtcaccgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagcggccgc

SEQ ID NO: 74 - DNA, 27 bp, Artificial Sequence
  Synthetic polynucleotide
cttcccggctgtctcacagtcctcagg

SEQ ID NO: 75 - DNA, 25 bp, Artificial Sequence
  Synthetic polynucleotide
gcaagatcgacttcctacggcggtg

SEQ ID NO: 76 - DNA, 725 bp, Artificial Sequence
  Synthetic polynucleotide
gtcgacatggattttcaggtgcagattatcagcttcctgctaatcagtgcttcagtcataatgtccagaggagacatcctgttgactcagagccctgtgatcctgtccgtgtcccctggcgagagggtctccttctcttgtcgagcgagccagtccatcggcaccaacatccactggtatcagcagaggaccaacgggtcccccaggctccttattaagtatgctagcgagagcatcagcggcatcccctccaggttctccggcagcggtagcggaaccgacttcaccctttctatcaactccgtggagagcgaggacatcgcggactattactgccagcagaataacaactggccaaccaccttcggcgcaggcactaagctggagctgaagcgcaccgtggcggctcccagcgtgttcatcttccccccgagcgacgagcagctgaaaagcggtactgcaagcgtcgtttgcctcctgaacaacttctaccccagggaggccaaggtgcagtggaaagtggacaacgccctgcagagcgggaacagccaagagtcagtgaccgagcaggactccaaggacagcacctacagcctcagctcaacgctgaccctgagcaaggccgattacgagaagcacaaggtgtacgcctgtgaggtgacccatcagggcctgtcttctcccgtcaccaaaagcttcaacagaggcgagtgctaagcggccgc

SEQ ID NO: 77 - DNA, 24 bp, Artificial Sequence
  Synthetic polynucleotide
ggagacatccagttgactcagagc

SEQ ID NO: 78 - DNA, 24 bp, Artificial Sequence
  Synthetic polynucleotide
gctctgagtcaactggatgtctcc

SEQ ID NO: 79 - DNA, 29 bp, Artificial Sequence
  Synthetic polynucleotide
actcagagcccttcgatcctgtccgtgtc

SEQ ID NO: 80 - DNA, 29 bp, Artificial Sequence
  Synthetic polynucleotide
gacacggacaggatcgaagggctctgagt

SEQ ID NO: 81 - DNA, 33 bp, Artificial Sequence
  Synthetic polynucleotide
gttgactcagagccctgtgagcctgtccgtgtc

SEQ ID NO: 82 - DNA, 33 bp, Artificial Sequence
  Synthetic polynucleotide
gacacggacaggctcacagggctctgagtcaac

SEQ ID NO: 83 - DNA, 31 bp, Artificial Sequence
  Synthetic polynucleotide
gttgactcagagcccttcgagcctgtccgtg

SEQ ID NO: 84 - DNA, 31 bp, Artificial Sequence
  Synthetic polynucleotide
cacggacaggctcgaagggctctgagtcaac

SEQ ID NO: 85 - DNA, 28 bp, Artificial Sequence
  Synthetic polynucleotide
tggacaacgccgatcagagcgggaacag

SEQ ID NO: 86 - DNA, 28 bp, Artificial Sequence
  Synthetic polynucleotide
ctgttcccgctctgatcggcgttgtcca

SEQ ID NO: 87 - DNA, 1433 bp, Artificial Sequence
  Synthetic polynucleotide
gtcgacatgggttggagcctcatcttgctcttccttgtcgctgttgctacgcgtgtcctgtcccaggtgcagctgaagcagagcggcccggggctcgtccagccctcgcagagcctgagcatcacctgcacggtgagcggcttcagcctgaccaactacggggtgcactgggtccggcagtcgcccggcaaggggctcgagtggctgggcgtgatctggagcggcgggaacaccgactacaacacccccttcacgagccgcctgagcatcaacaaggacaacagcaagtcgcaggtgttcttcaagatgaacagcctccagagcaacgacaccgccatctactactgcgcgcgggccctgacctactacgactacgagttcgcctactggggccaggggaccctggtcacggtgagcgccgcgagcaccaagggcccgagcgtgttccccctcgccccctcgagcaagagcaccagcggcgggaccgccgccctgggctgcctggtcaaggactacttccccgagccggtgacggtgagctggaactcgggggccctcaccagcggcgtccacaccttccccgcggtgctgcagagcagcgggctgtacagcctcagctcggtggtcaccgtgcccagcagcagcctgggcacgcagacctacatctgcaacgtgaaccacaagcccagcaacaccaaggtcgacaagcgcgtggagccgaagtcgcccaagagctgcgacaagacccacacgtgcccgccctgccccgcccccgagctgctcggcgggcccagcgtgttcctgttcccgcccaagcccaaggacaccctgatgatcagccggacccccgaggtcacctgcgtggtggtcgacgtgagccacgaggatccggaggtgaagttcaactggtacgtcgacggcgtggaggtgcacaacgccaagacgaagccccgcgaggagcagtacaacagcacctaccgggtcgtgtcggtgctcaccgtcctgcaccaggactggctgaacgggaaggagtacaagtgcaaggtgagcaacaaggccctccccgcgcccatcgagaagaccatcagcaaggccaagggccagccgcgcgagccccaggtgtacacgctgccccccagccgggacgagctgaccaagaaccaggtcagcctcacctgcctggtgaaggggttctacccgtcggacatcgccgtggagtgggagagcaacggccagcccgagaacaactacaagaccacgcccccggtcctggacagcgacggcagcttcttcctctacagcaagctgaccgtggacaagagccgctggcagcaggggaacgtgttctcgtgcagcgtcatgcacgaggccctgcacaaccactacacccagaagagcctcagcctgagccccggcaagtgataagcggccgc

SEQ ID NO: 88 - protein, 224 aa, Artificial Sequence
  Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTXYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 89 - protein, 224 aa, Artificial Sequence
  Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTHYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 90 - protein, 213 aa, Artificial Sequence
  Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKKYACEVTHQGLSSPVTKSFNRGEC

SEQ ID NO: 91 - protein, 213 aa, Artificial Sequence
  Synthetic polypeptide
QIVLSQSPAKLSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 213 aa, Artificial Sequence
  Synthetic polypeptide
QIVLSQSPAILSASPGEKVTMTCRASSSVSYIHWFQQKPGSSPKPWIYATSNLASGVPDRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 213 aa, Artificial Sequence
  Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTKAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 224 aa, Artificial Sequence
  Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVKQSSGLYSLSSVVTVPSSSLGTQTYICNVNHKPSNTKVDKKVEPKSC

SEQ ID NO: 95 - protein, 220 aa, Artificial Sequence
  Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARXTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKS

SEQ ID NO: 96 - protein, 221 aa, Artificial Sequence
  Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTXVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKS

SEQ ID NO: 97 - protein, 214 aa, Artificial Sequence
  Synthetic polypeptide
DIKLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 214 aa, Artificial Sequence
  Synthetic polypeptide
DILLTQSPDILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 214 aa, Artificial Sequence
  Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNAKQSGNSQESVTEQDSKDSTYSLSSTLTLSKADYEKHKVYACEVTHQGLSSPVTKSFNRGEC

SEQ ID NO: 100 - protein, 221 aa, Artificial Sequence
  Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARAKTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKS

SEQ ID NO: 101 - protein, 221 aa, Artificial Sequence
  Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTKVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKS

SEQ ID NO: 102 - protein, 221 aa, Artificial Sequence
  Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVKQSSGLYSLSSVVTVPSSSLGTQTYICNVNHKPSNTKVDKRVEPKS

(19) United States
(12) Patent Application Publication
(10) Pub. No.: US 2015/0239977 A1
(43) Pub. Date: Aug. 27, 2015

(54) CD20- AND EGFR-BINDING PROTEINS WITH ENHANCED STABILITY

(71) Applicant: MASSACHUSETTS INSTITUTE OF TECHNOLOGY, Cambridge, MA (US)

(72) Inventors: Bernhardt Levy Trout, Lexington, MA (US); Curtiss Paul Schneider, Melrose, MA (US); Neeraj Jagdish Agrawal, Thousand Oaks, CA (US)

(73) Assignee: Massachusetts Institute of Technology, Cambridge, MA (US)

(21) Appl. No.: 14/431,830

(22) PCT Filed: Sep. 27, 2013
(86) PCT No.: PCT/US13/62114

§ 371 Date: Mar. 27, 2015

Related U.S. Application Data
(60) Provisional application No. 61/706,242, filed on Sep. 27, 2012.
(60) Provisional application No. 61/830,832, filed on Jun. 4, 2013.

(51) Int. Cl. C07K 16/28; A61J 1/06

(52) U.S. Cl. CPC C07K 16/2887; C07K 16/2863; A61J 1/065; C07K 2317/51; C07K 2317/515; C07K 2317/52

(57) ABSTRACT

The invention provides CD20-binding proteins and EGFR-binding proteins having a reduced tendency to aggregate. Compositions and methods of use are also provided.

### RELATED APPLICATIONS

This application claims the benefit under 35 U.S.C. §119(e) of U.S. provisional application numbers 61/830,832, filed Jun. 4, 2013, and 61/706,242, filed Sep. 27, 2012, which are incorporated by reference herein.

### FIELD OF THE INVENTION

Aspects of the invention relate to proteins that are less prone to aggregation as compared to existing proteins and that may be suitable for preparing highly concentrated protein compositions.

### BACKGROUND OF INVENTION

Many therapeutic proteins such as, for example, antibodies require administration by injection or infusion via the intravenous route. The amount of protein that can be administered intravenously is limited by solubility and stability of the protein in a suitable liquid composition and by the volume of the infusion fluid. An alternative administration pathway is subcutaneous injection. This injection pathway requires a high protein concentration in the final solution to be injected (Shire et al., Journal of Pharmaceutical Science, 2004; 93(6): 1390-1402; Roskos et al., Drug Development Research, 2004; 61(3): 108-120). Achieving the high protein concentration necessary for subcutaneous delivery can be problematic due to protein aggregation. Aggregation is the result of intermolecular interactions and, thus, is enhanced by high protein concentrations. The presence of protein aggregates in an injected solution, even in small doses, poses a threat of an immune response that can reduce the efficacy of the protein over time and, more importantly, has the potential to elicit adverse reactions (Frokjaer, S. et al., Nature Reviews Drug Discovery, 2005; 4(4): 298-306; Wang, W., et al., International Journal of Pharmaceutics, 2005; 289: 1-30; Manning, M. C., et al., Pharmaceutical Research, 2010; 27(4): 544-575; Cromwell, M. E. M., et al., 2006; Rosenberg, A. S., AAPS J., 2006; 8(3): E501-E507).

### SUMMARY OF INVENTION

The invention provides, inter alia, proteins that are less prone to aggregation as compared to existing proteins. The invention is based, in part, on the surprising discovery that specific mutations at particular amino acids in CD20-binding proteins and in EGFR-binding proteins can reduce the tendency of the proteins to aggregate in solution.

Thus, in various aspects and embodiments of the invention, provided herein are CD20-binding proteins comprising a light chain domain having an amino acid sequence of SEQ ID NO:1 modified to include at least one amino acid substitution selected from a neutral polar amino acid at V₃, a neutral polar amino acid at A₉, a neutral polar amino acid at I₁₀, a positive polar amino acid at I₁₀, a neutral polar amino acid at V₅₉, a negative polar amino acid at V₅₉, a positive polar amino acid at V₁₀₉, and a negative polar amino acid at L₁₅₃; and/or a heavy chain domain having an amino acid sequence of SEQ ID NO:2 modified to include at least one amino acid substitution selected from a neutral polar amino acid at Y₁₀₁, a neutral polar amino acid at L₁₇₈, and a positive polar amino acid at L₁₇₈; and/or an Fc domain having an amino acid sequence of SEQ ID NO:3 modified to include at least one amino acid substitution selected from a positive polar amino acid at position L₁₅, a positive polar amino acid at position I₃₃, a positive polar amino acid at position V₆₂, and a positive polar amino acid at position L₈₉. Also provided herein are compositions comprising the CD20-binding proteins and methods of use.

Non-limiting examples of CD20-binding protein amino acid substitutions contemplated by the invention are presented in Table I.

TABLE I
Examples of CD20-binding protein amino acid substitutions.

| Full length, wild type | |
|---|---|
| sequence | Contemplated S = substitutions |
| light chain domain | V3 (SEQ ID NO: 19) - substitution |
| (SEQ ID NO: 1) | to a neutral polar amino acid |
| | V3Q (SEQ ID NO: 4) |
| | A9 (SEQ ID NO: 20) - substitution |
| | to a neutral polar amino acid |
| | A9S (SEQ ID NO: 5) |
| | I10 (SEQ ID NO: 21) - substitution |
| | to a neutral polar amino acid or a |
| | positive polar amino acid |
| | I10S (SEQ ID NO: 6) |
| | I10K (SEQ ID NO: 91) |
| | V59 (SEQ ID NO: 22) - substitution |
| | to a neutral polar amino acid or a |
| | negative polar amino acid |
| | V59S (SEQ ID NO: 7) |
| | V59D (SEQ ID NO: 92) |
| | V109 (SEQ ID NO: 90) - substitution |
| | to a positive polar amino acid |
| | V109K (SEQ ID NO: 93) |
| | L153 (SEQ ID NO: 23) - substitution |
| | to a negative polar amino acid |
| | L153D (SEQ ID NO: 8) |
| Combinations | A9 and I10 (SEQ ID NO: 25) |
| | A9S and I10S (SEQ ID NO: 10) |
| | V3, A9 and V59 (SEQ ID NO: 26) |
| | V3Q, A9S and V59S (SEQ ID NO: 11) |
| | V3, A9, I10 and V59 (SEQ ID NO: 27) |
| | V3Q, A9S, I10S and V59S |
| | (SEQ ID NO: 12) |
| | V3, A9, V59 and L153 (SEQ ID NO: 28) |
| | V3Q, A9S, V59S and L153D |
| | (SEQ ID NO: 13) |
| | V3, A9, I10, V59 and L153 |
| | (SEQ ID NO: 29) |
| | V3Q, A9S, I10S, V59S and L153D |
| | (SEQ ID NO: 14) |
| Fab heavy chain domain | Y101 (SEQ ID NO: 88) - substitution |
| (SEQ ID NO: 2) | to a neutral polar amino acid |
| | Y101H (SEQ ID NO: 89) |
| | L178 (SEQ ID NO: 24)- substitution |
| | to a neutral polar amino acid or a |
| | positive polar amino acid |
| | L178S (SEQ ID NO: 9) |
| | L178K (SEQ ID NO: 94) |
| Fc domain | L15 (SEQ ID NO: 30) - substitution |
| (SEQ ID NO: 3) | to a positive polar amino acid |
| | L15K (SEQ ID NO: 15) |
| | I33 (SEQ ID NO: 31) - substitution |
| | to a positive polar amino acid |
| | I33K (SEQ ID NO: 16) |
| | V62 (SEQ ID NO: 32) - substitution |
| | to a positive polar amino acid |
| | V62K (SEQ ID NO: 17) |
| | L89 (SEQ ID NO: 33) - substitution |
| | to a positive polar amino acid |
| | L89K (SEQ ID NO: 18) |

In some embodiments, the CD20-binding proteins comprise a light chain domain having an amino acid sequence of SEQ ID NO:1 modified to include at least two, at least three, at least four, at least five, or at least six amino acid substitutions selected from a neutral polar amino acid at V₃, a neutral polar amino acid at A₉, a neutral polar amino acid at I₁₀, a positive polar amino acid at I₁₀, a neutral polar amino acid at V₅₉, a negative polar amino acid at V₅₉, a positive polar amino acid at V₁₀₉, and a negative polar amino acid at L₁₅₃; and/or a heavy chain domain having an amino acid sequence of SEQ ID NO:2, or a heavy chain domain having an amino acid sequence of SEQ ID NO:2 modified to include at least two amino acid substitutions selected from a neutral polar amino acid at Y₁₀₁, a neutral polar amino acid at L₁₇₈, and a positive polar amino acid at L₁₇₈.

In some embodiments, the neutral polar amino acid at V₃, A₉, I₁₀ and/or V₅₉ of SEQ ID NO:1 and/or at Y₁₀₁ and/or L₁₇₈ of SEQ ID NO:2 may be selected from asparagine (N), cysteine (C), glutamine (Q), histidine (H), serine (S), threonine (T) and tyrosine (Y). In some embodiments, the negative polar amino acid at V₅₉ and/or L₁₅₃ of SEQ ID NO:1 may be aspartic acid (D) or glutamic acid (E). In some embodiments, the positive polar amino acid at I₁₀ and/or V₁₀₉ of SEQ ID NO:1, at L₁₇₈ of SEQ ID NO:2, and/or at L₁₅, I₃₃, V₆₂ and/or L₈₉ of SEQ ID NO:3 may be R or K.

In some embodiments, the CD20-binding proteins comprise a light chain domain having an amino acid sequence of SEQ ID NO:1, a heavy chain domain having an amino acid sequence of SEQ ID NO:2, a human IgG Fc domain, or an Fc domain having an amino acid sequence of SEQ ID NO:3.

In some embodiments, the CD20-binding proteins comprise a light chain domain having an amino acid sequence of SEQ ID NO:19, SEQ ID NO:20, SEQ ID NO:21, SEQ ID NO:22, SEQ ID NO:23, or SEQ ID NO:90, where position 3 of SEQ ID NO:19, position 9 of SEQ ID NO:20, position 10 of SEQ ID NO: 21 and/or position 59 of SEQ ID NO:22 is optionally modified to a neutral polar amino acid, where position 153 of SEQ ID NO:23 and/or position 59 of SEQ ID NO:22 is optionally modified to a negative polar amino acid, and where position 10 of SEQ ID NO:21 and/or position 109 of SEQ ID NO:90 is optionally modified to a positive polar amino acid.

In some embodiments, the CD20-binding proteins comprise a light chain domain having an amino acid sequence of SEQ ID NO:25, where positions 9 and 10 are optionally modified to a neutral polar amino acid. In some embodiments, the CD20-binding proteins comprise a light chain domain having an amino acid sequence of SEQ ID NO:26, where positions 3, 9 and 59 are optionally modified to a neutral polar amino acid. In some embodiments, the CD20-binding proteins comprise a light chain domain having an amino acid sequence of SEQ ID NO:27, where positions 3, 9, 10 and 59 are optionally modified to a neutral polar amino acid. In some embodiments, the CD20-binding proteins comprise a light chain domain having an amino acid sequence of SEQ ID NO:28, where positions 3, 9 and 59 are optionally modified to a neutral polar amino acid and position 153 is optionally modified to a negative polar amino acid. In some embodiments, the CD20-binding proteins comprise a light chain domain having an amino acid sequence of SEQ ID NO:29. where positions 3, 9, 10 and 59 are optionally modified to a neutral polar amino acid and position 153 is optionally modified to a negative polar amino acid.

In some embodiments, the CD20-binding proteins comprise a heavy chain domain having an amino acid sequence of SEQ ID NO:88, where position 101 is optionally modified to a neutral amino acid. In some embodiments, the CD20-binding proteins comprise a heavy chain domain having an amino acid sequence of SEQ ID NO:24, where position 178 is optionally modified to a neutral polar amino acid or a positive polar amino acid.

In some embodiments, the CD20-binding proteins comprise an Fc chain domain having an amino acid sequence of SEQ ID NO:30, SEQ ID NO:31, SEQ ID NO:32 or SEQ ID NO:33, where position 15 of SEQ ID NO:30, position 33 of SEQ ID NO:31, position 62 of SEQ ID NO:32 and position 89 of SEQ ID NO:33 is optionally modified to a positive polar amino acid.

In some aspects of the invention, provided herein are CD20-binding proteins comprising a light chain domain having an amino acid sequence of SEQ ID NO:1 modified to include at least one amino acid substitution selected from V₃Q, A₉S, I₁₀S, I₁₀K, V₅₉S, V₅₉D V₁₀₉K and L₁₅₃D; and/or a heavy chain domain having an amino acid sequence of SEQ ID NO:2 modified to include at least one amino acid substitution selected from Y₁₀₁H, L₁₇₈S and L₁₇₈K. In some embodiments, the CD20-binding proteins comprise a light chain domain having an amino acid sequence of SEQ ID NO:1 modified to include at least one amino acid substitution selected from the group consisting of V₃K, A₉K, I₁₀K, V₅₉K, V₁₀₉K and L₁₅₃K; and/or a heavy chain domain having an amino acid sequence of SEQ ID NO:2 modified to include at least one amino acid substitution selected from the group consisting of Y₁₀₁H, L₁₇₈K and L₁₇₈S.

In some embodiments, the CD20-binding proteins comprise a light chain domain having an amino acid sequence of SEQ ID NO:1 modified to include at least two, at least three, at least four, at least five, or at least six amino acid substitutions selected from the group consisting of V₃Q, A₉S, I₁₀S, I₁₀K, V₅₉S, V₅₉D, V₁₀₉K and L₁₅₃D. In some embodiments, the CD20-binding proteins comprise a light chain domain having an amino acid sequence of SEQ ID NO:1 modified to include at least two, at least three, at least four, at least five, or at least six amino acid substitutions selected from the group consisting of V₃K, A₉K, I₁₀K, V₅₉K, V₁₀₉K and L₁₅₃K. In some embodiments, the CD20-binding proteins comprise a heavy chain domain having an amino acid sequence of SEQ ID NO:2 modified to include an amino acid substitution Y₁₀₁H and an amino acid substitution selected from L₁₇₈S and L₁₇₈K.

In some embodiments, the CD20-binding proteins comprise a light chain domain having an amino acid sequence of SEQ ID NO:1 modified to include amino acid substitutions selected from (a) A₉S and I₁₀S, (b) V₃Q, A₉S and V₅₉S, (c) V₃Q, A₉S, I₁₀S and V₅₉S, (d) V₃Q, A₉S, V₅₉S and L₁₅₃D, and (e) V₃Q, A₉S, I₁₀S, V₅₉S and L₁₅₃D. In some embodiments, the CD20-binding proteins comprise a light chain domain having an amino acid sequence of SEQ ID NO:4, SEQ ID NO:5, SEQ ID NO:6, SEQ ID NO:7, SEQ ID NO:8, SEQ ID NO:91, SEQ ID NO:92, SEQ ID NO:93, SEQ ID NO:10, SEQ ID NO:11, SEQ ID NO:12, SEQ ID NO:13, SEQ ID NO:14, SEQ ID NO:19, SEQ ID NO:20, SEQ ID NO:21, SEQ ID NO:22, SEQ ID NO:90, SEQ ID NO:23, SEQ ID NO:25, SEQ ID NO:26, SEQ ID NO:27, SEQ ID NO:28, or SEQ ID NO:29.

In some embodiments, the CD20-binding proteins comprise a heavy chain domain having an amino acid sequence of SEQ ID NO:88, SEQ ID NO:24, SEQ ID NO: 89, SEQ ID NO:9 or SEQ ID NO:94.

In some embodiments, the CD20-binding proteins comprise a light chain domain having an amino acid sequence of SEQ ID NO:1 modified to include at least one amino acid substitution selected from a neutral polar amino acid at V₃, a neutral polar amino acid at A₉, a neutral polar amino acid at I₁₀, a positive polar amino acid at I₁₀, a neutral polar amino acid at V₅₉, a negative polar amino acid at V₅₉, a positive polar amino acid at V₁₀₉, and a negative polar amino acid at L₁₅₃; and a heavy chain domain having an amino acid sequence of SEQ ID NO:2. In some embodiments, the neutral polar amino acid at V₃, A₉, I₁₀ and/or V₅₉ of SEQ ID NO:1 and/or at Y₁₀₁ and/or L₁₇₈ of SEQ ID NO:2 may be selected from N, C, Q, H, S, T and Y. In some embodiments, the negative polar amino acid at V₅₉ and/or L₁₅₃ of SEQ ID NO:1 may be D or E. In some embodiments, the positive polar amino acid at I₁ and/or V₁₀₉ of SEQ ID NO:1, at L₁₇₈ of SEQ ID NO:2, and/or at L₁₅, I₃₃, V₆₂ and/or L₈₉ of SEQ ID NO:3 may be R or K. In some embodiments, the CD20-binding proteins comprise a light chain domain having an amino acid sequence of SEQ ID NO:1, and a heavy chain domain having an amino acid sequence of SEQ ID NO:2 modified to include at least one amino acid substitution selected from a neutral polar amino acid at Y₁₀₁, a neutral polar amino acid at L₁₇₈, and a positive polar amino acid at L₁₇₈. In some embodiments, the neutral polar amino acid at Y₁₀₁ and/or L₁₇₈ of SEQ ID NO:2 may be selected from N, C, Q, H, S, T and Y. In some embodiments, the positive polar amino acid at L₁₇₈ may be R or K.

In some embodiments, the CD20-binding proteins comprise a human IgG1 Fc domain. In some embodiments, the CD20-binding proteins comprise an Fc domain having an amino acid sequence of SEQ ID NO:3. In some embodiments, the Fc domain having the amino acid sequence of SEQ ID NO:3 is modified to include at least one amino acid substitution selected from L₁₅, I₃₃, V₆₂ and L₈₉. In some embodiments, the Fc domain having the amino acid sequence of SEQ ID NO:3 is modified to include at least one amino acid substitution selected from a positive polar amino acid at L₁₅K, I₃₃K, V₆₂K and L₈₉K.

In some embodiments, the CD20-binding proteins comprise an Fc domain having an amino acid sequence of SEQ ID NO:15, SEQ ID NO:16, SEQ ID NO:17, SEQ ID NO:18, SEQ ID NO:30, SEQ ID NO:31, SEQ ID NO:32 or SEQ ID NO:33.

In some aspects of the invention, provided herein are CD20-binding proteins comprising an Fc domain having an amino acid sequence of SEQ ID NO:3 modified to include at least one amino acid substitution selected from a positive polar amino acid at position L₁₅, a positive polar amino acid at position I₃₃, a positive polar amino acid at position V₆₂, and a positive polar amino acid at position L₈₉. In some embodiments, the positive polar amino acid may be R or K. In some embodiments, the CD20-binding proteins comprise an Fc domain having an amino acid sequence of SEQ ID NO:3 modified to include at least one amino acid substitution selected from L₁₅K, I₃₃K, V₆₂K and L₈₉K.

In some embodiments, the CD20-binding proteins comprise an Fc domain having an amino acid sequence of SEQ ID NO:3 modified to include at least one amino acid substitution selected from a positive polar amino acid at position L₁₅, a positive polar amino acid at position I₃₃, a positive polar amino acid at position V₆₂, and a positive polar amino acid at position L₈₉; a light chain domain having an amino acid sequence of SEQ ID NO:1; and a heavy chain domain having an amino acid sequence of SEQ ID NO:2.

In some embodiments, the CD20-binding proteins comprise an Fc domain having an amino acid sequence of SEQ ID NO:3 modified to include at least one amino acid substitution selected from a positive polar amino acid at position L₁₅, a positive polar amino acid at position I₃₃, a positive polar amino acid at position V₆₂, and a positive polar amino acid at position L₈₉; a light chain domain having an amino acid sequence of SEQ ID NO:1 modified to include at least one amino acid substitution selected from the group consisting of a neutral polar amino acid at V₃, a neutral polar amino acid at A₉, a neutral polar amino acid at I₁₀, a positive polar amino acid at I₁₀, a neutral polar amino acid at V₅₉, a negative polar amino acid at V₅₉, a positive polar amino acid at V₁₀₉, and a negative polar amino acid at L₁₅₃; and a heavy chain domain having an amino acid sequence of SEQ ID NO:2.

In some embodiments, the CD20-binding proteins comprise an Fc domain having an amino acid sequence of SEQ ID NO:3 modified to include at least one amino acid substitution selected from a positive polar amino acid at position L₁₅, a positive polar amino acid at position I₃₃, a positive polar amino acid at position V₆₂, and a positive polar amino acid at position L₈₉; a light chain domain having an amino acid sequence of SEQ ID NO:1; and heavy chain domain having an amino acid sequence of SEQ ID NO:2 modified to include at least one amino acid substitution selected from the group consisting of an neutral polar amino acid at Y₁₀₁, a neutral polar amino acid at L₁₇₈, and a positive polar amino acid at L₁₇₈.

In some embodiments, the CD20-binding proteins are in the form of monoclonal antibodies such as, for example, chimeric monoclonal antibodies or humanized monoclonal antibodies. In some embodiments, the CD20-binding proteins are in the form of antigen-binding antibody fragments such as, for example, Fab antibody fragments. In some embodiments, the CD20-binding proteins are in the form of antibody-like proteins such as, for example, fusion proteins, single-chain Fv antibody fragments, and/or minibodies. In some embodiments, the CD20-binding proteins are conjugated to a therapeutic or diagnostic agent such as a toxin and/or a radioisotope.

In some embodiments, the CD20-binding proteins are lyophilized. In some embodiments, the CD20-binding proteins are in solution.

In some aspects of the invention, provided herein are compositions comprising any of the CD20-binding proteins of the invention. In some embodiments, the compositions comprise CD20-binding proteins comprising: a light chain domain having an amino acid sequence of SEQ ID NO:1 modified to include at least one amino acid substitution selected from a neutral polar amino acid at V₃, a neutral polar amino acid at A₉, a neutral polar amino acid at I₁₀, a positive polar amino acid at I₁₀, a neutral polar amino acid at V₅₉, a negative polar amino acid at V₅₉, a positive polar amino acid at V₁₀₉, and a negative polar amino acid at L₁₅₃; and/or a heavy chain domain having an amino acid sequence of SEQ ID NO:2 modified to include at least one amino acid substitution selected from a neutral polar amino acid at Y₁₀₁, a neutral polar amino acid at L₁₇₈, and a positive polar amino acid at L₁₇₈.

In some embodiments, the compositions comprise CD20-binding proteins comprising a light chain domain having an amino acid sequence of SEQ ID NO:1 modified to include at least one amino acid substitution selected from V₃Q, A₉S, I₁₀S, I₁₀K, V₅₉S, V₅₉D, V₁₀₉K and L₁₅₃D; and/or a heavy chain domain having an amino acid sequence of SEQ ID NO:2 modified to include at least one amino acid substitution selected from a neutral polar amino acid at Y₁₀₁, a neutral polar amino acid at L₁₇₈, and a positive polar amino acid at L₁₇₈.

In some embodiments, the compositions comprise CD20-binding proteins comprising an Fc domain having an amino acid sequence of SEQ ID NO:3 modified to include at least one amino acid substitution selected from a positive polar amino acid at position L₁₅, a positive polar amino acid at position I₃₃, a positive polar amino acid at position V₆₂, and a positive polar amino acid at position L₈₉. In some embodiments, the compositions comprise CD20-binding proteins comprising an Fc domain having an amino acid sequence of SEQ ID NO:3 modified to include at least one amino acid substitution selected from L₁₅K, I₃₃K, V₆₂K and L₈₉K.

In some embodiments, the compositions comprise the CD20-binding proteins at a concentration of about 20 mg/ml to about 350 mg/ml. In some embodiments, the concentration of the CD20-binding proteins is about 100 mg/ml to about 250 mg/ml. In some embodiments, the concentration of the CD20-binding proteins is about 120 mg/ml to about 150 mg/ml. In some embodiments, the concentration of the CD20-binding proteins is about 120 mg/ml, about 130 mg/ml or about 140 mg/ml. In some embodiments, the concentration of the CD20-binding proteins is about 120 mg/ml or is 120 mg/ml.

In some embodiments, the compositions are liquid. In some embodiments, the compositions are formulated for subcutaneous injection and/or other non-intravenous delivery routes such as, for example, intramuscular injection.

In some embodiments, the compositions further comprise at least one buffer, at least one stabilizer and/or at least one surfactant. In some embodiments, the buffer is present at a concentration of about 1 mM to about 100 mM. In some embodiments, the buffer provides a pH of 5.5±2.0. In some embodiments, the stabilizer is present at a concentration of about 100 mM to about 500 mM. In some embodiments, a secondary stabilizer is present. In some embodiments, the secondary stabilizer is arginine, ornithine, lysine, histidine, glutamic acid, aspartic acid, isoleucine, leucine, alanine, phenylalanine, tyrosine, tryptophan, methionine, serine or proline (e.g., as a hydrochloride). In some embodiments, methionine is present at a concentration of about 5 mM to about 25 mM. In some embodiments, the surfactant is a nonionic surfactant. In some embodiments, the surfactant is present at a concentration of about 0.01% to about 0.1%. In some embodiments, the compositions further comprise at least one pharmaceutically acceptable carrier, excipient, and/or diluent. In some embodiments, the compositions further comprise additional agents (e.g., therapeutic agents). In some embodiments, the compositions are sterile.

In some embodiments, the compositions comprise the CD20-binding proteins, histidine HCl, trehalose dehydrate, methionine and/or polysorbate 80. In some embodiments, the compositions comprise about 100 mg/ml to about 150 mg/ml of the CD20-binding proteins, about 10 mM to about 30 mM histidine HCl, about 200 mM to about 220 mM trehalose dehydrate, about 5 mM to about 15 mM methionine and/or about 0.04% to about 0.08% polysorbate 80. In some embodiments, the compositions comprise about 120 mg/ml of the CD20-binding proteins, about 20 mM histidine HCl, about 210 mM trehalose dehydrate, about 10 mM methionine and/or about 0.06% polysorbate 80.

In some aspects of the invention, provided herein are methods of treating a condition in a subject in need thereof, comprising administering to the subject any of the CD20-binding proteins and/or compositions described herein. In some embodiments, the methods comprise administering to the subject a therapeutically effective amount of any of the CD20-binding proteins and/or compositions provided herein. In some embodiments, the condition is cancer or a non-malignant condition. In some embodiments, the condition involves CD20-expressing cells. In some embodiments, the cancer is a human cancer such as, for example, B cell lymphomas and leukemias, and other cancers expressing or overespressing CD20. In some embodiments, the non-malignant condition is an autoimmune condition such as, for example, rheumatoid arthritis.

In some aspects of the invention, provided herein are uses of the CD20-binding proteins for the preparation of a medicament for treating a condition amenable to treatment with a CD20-binding protein.

In some aspects of the invention, provided herein are nucleic acids encoding the CD20-binding proteins, vectors comprising the nucleic acids, expression cassettes comprising the nucleic acids, and host cells comprising the nucleic acids and/or vectors and/or expression cassettes. In some embodiments, the nucleic acids are isolated.

In some aspects of the invention, provided herein are methods of producing a CD20-binding protein (e.g., an anti-CD20 antibody), the methods comprising culturing any of the host cells described herein to produce the CD20-binding protein. In certain embodiments, the methods include an additional step of isolating the CD20-binding protein.

In some aspects of the invention, provided herein are kits comprising a container and any of the CD20-binding proteins and/or compositions contained therein. In some embodiments, the kits further comprise instructions for using the kits. In some embodiments, the kits further comprise a package insert or label indicating that the kits can be used to treat cancer or a non-malignant condition such as, for example, one characterized by the overexpression of CD20. In some embodiments, the kits comprise injection components such as, for example, a syringe or a syringe filled with the CD20-binding proteins or a composition containing the CD20-binding proteins. In some embodiments, the volume of the composition in the kits is about 2 ml. In some embodiments, the volume of the composition is less than 2 ml. In some embodiments, the concentration of the CD20-binding proteins in about 2 ml or less of a composition is about 100 mg/ml to about 150 mg/ml. In some embodiments, the concentration of the CD20-binding proteins is about 120 mg/ml or is 120 mg/ml.

In various other aspects the invention, provided herein are epidermal growth factor receptor (EGFR)-binding proteins comprising a light chain domain having an amino acid sequence of SEQ ID NO:34 modified to include at least one amino acid substitution selected from a neutral polar amino acid at L₃, a positive polar amino acid at L₃, a neutral polar amino acid at V₉, a negative polar amino acid at V₉, a neutral polar amino acid at I₁₀, a negative polar amino acid at L₁₅₄, and a positive polar amino acid at L₁₅₄; and/or a heavy chain domain having an amino acid sequence of SEQ ID NO:35 modified to include at least one amino acid substitution selected from a positive polar amino acid at L₉₉, a positive polar amino acid at L₁₁₄, a neutral polar amino acid at L₁₇₆, and a positive polar amino acid at L₁₇₆. Also provided herein are compositions comprising the EGFR-binding proteins and methods of use. In some embodiments, the EGFR-binding proteins comprise a light chain domain having an amino acid sequence of SEQ ID NO:34 modified to include at least two, at least three, or at least four amino acid substitutions selected from a neutral polar amino acid at L₃, a positive polar amino acid at L₃, a neutral polar amino acid at V₉, a negative polar amino acid at V₉, a neutral polar amino acid at I₁₀, a negative polar amino acid at L₁₅₄, and a positive polar amino acid at L₁₅₄; and/or a heavy chain domain having an amino acid sequence of SEQ ID NO:35, or a heavy chain domain having an amino acid sequence of SEQ ID NO:35 modified to include at least two or at least three amino acid substitutions selected from a positive polar amino acid at L₉₉, a positive polar amino acid at L₁₁₄, a neutral polar amino acid at L₁₇₆, and a positive polar amino acid at L₁₇₆. Non-limiting examples of EGFR-binding protein amino acid substitutions contemplated by the invention are presented in Table II.

TABLE II
Examples of EGFR-binding protein amino acid substitutions.

| Full length, wild type | |
|---|---|
| sequence | Contemplated substitutions |
| light chain domain | L3(SEQ ID NO: 51) - substitution |
| (SEQ ID NO: 34) | to a neutral polar amino acid or a |
| | positive polar amino acid |
| | L3Q (SEQ ID NO: 37) |
| | L3K(SEQ ID NO: 97) |
| | V9 (SEQ ID NO: 52) - substitution |
| | to a neutral polar amino acid or a |
| | negative polar amino acid |
| | V9S (SEQ ID NO: 38) |
| | V9D (SEQ ID NO: 98) |
| | I10 (SEQ ID NO: 53) - substitution |
| | to a neutral polar amino acid |
| | I10S (SEQ ID NO: 39) |
| | L154 (SEQ ID NO: 54) - substitution |
| | to a negative polar amino acid or |
| | a positive polar amino acid |
| | L154D (SEQ ID NO: 40) |
| | L154K (SEQ ID NO: 99) |
| Combinations | V9 and I10 (SEQ ID NO: 56) |
| | V9S and I10S (SEQ ID NO: 42) |
| | L3 and V9 (SEQ ID NO: 57) |
| | L3Q and V9S (SEQ ID NO: 43) |
| | L3, V9 and I10 (SEQ ID NO: 58) |
| | L3Q, V9S and I10S |
| | (SEQ ID NO: 44) |
| | L3, V9 and L154 (SEQ ID NO: 59) |
| | L3Q, V9S and L154D (SEQ ID NO: 45) |
| | L3, V9, I10 and L154 ( |
| | SEQ ID NO: 60) |
| | L3Q, V9S, I10S and L154D |
| | (SEQ ID NO: 46) |
| Fab heavy chain domain | L99 (SEQ ID NO: 95) - substitution |
| (SEQ ID NO: 35) | to a positive polar amino acid |
| | L99K (SEQ ID NO: 100) |
| | L114 (SEQ ID NO: 96) - substitution |
| | to a positive polar amino acid |
| | L114K (SEQ ID NO: 101) |
| | L176 (SEQ ID NO: 55) - substitution |
| | to a neutral polar amino acid or a |
| | positive polar amino acid |
| | L176S (SEQ ID NO: 41) |
| | L176K (SEQ ID NO: 102) |
| Fc domain | L19 (SEQ ID NO: 61) - substitution |
| (SEQ ID NO: 36) | to a positive polar amino acid |
| | L19K (SEQ ID NO: 47) |
| | 137 (SEQ ID NO: 62) - substitution |
| | to a positive polar amino acid |
| | I37K (SEQ ID NO: 48) |
| | V66 (SEQ ID NO: 63) - substitution |
| | to a positive polar amino acid |
| | V66K (SEQ ID NO: 49) |
| | L93 (SEQ ID NO: 64) - substitution |
| | to a positive polar amino acid |
| | L93K (SEQ ID NO: 50) |

In some embodiments, the neutral polar amino acid at L₃, V₉ and/or I₁₀ of SEQ ID NO:34 and/or at L₁₇₆ of SEQ ID NO:35 may be selected from (N), cysteine (C), glutamine (Q), histidine (H), serine (S), threonine (T) and tyrosine (Y). In some embodiments, the negative polar amino acid at L₁₅₄ of SEQ ID NO:34 may be aspartic acid (D) or glutamic acid (E). In some embodiments, the positive polar amino acid at L₃ and/or L₁₅₄ of SEQ ID NO:34, at L₉₉, L₁₁₄ and/or L₁₇₆ of SEQ ID NO:35, and/or at L₁₉, I₃₇, V₆₆ and/or L₉₃ of SEQ ID NO:36 may be arginine (R) or lysine (K).

In some embodiments, the EGFR-binding proteins comprise a light chain domain having an amino acid sequence of SEQ ID NO:34, a heavy chain domain having an amino acid sequence of SEQ ID NO:35, a human IgG Fc domain, or an Fc domain having an amino acid sequence of SEQ ID NO:36.

In some embodiments, the EGFR-binding proteins comprise a light chain domain having an amino acid sequence of SEQ ID NO:51, wherein position 3 is optionally modified to a neutral polar or a positive polar amino acid. In some embodiments, the EGFR-binding proteins comprise a light chain domain having an amino acid sequence of SEQ ID NO:52, wherein position 9 is optionally modified to a neutral polar or a negative polar amino acid. In some embodiments, the EGFR-binding proteins comprise a light chain domain having an amino acid sequence of SEQ ID NO:53, wherein position 10 is optionally modified to a neutral polar amino acid. In some embodiments, the EGFR-binding proteins comprise a light chain domain having an amino acid sequence of SEQ ID NO:54, wherein position 154 is optionally modified to a negative polar or a positive polar amino acid.

In some embodiments, the EGFR-binding proteins comprise a light chain domain having an amino acid sequence of SEQ ID NO:56, where positions 9 and 10 are optionally modified to a neutral polar amino acid. In some embodiments, the EGFR-binding proteins comprise a light chain domain having an amino acid sequence of SEQ ID NO:57, where positions 3 and 9 are optionally modified to a neutral polar amino acid. In some embodiments, the EGFR-binding proteins comprise a light chain domain having an amino acid sequence of SEQ ID NO:58, where positions 3, 9 and 10 are optionally modified to a neutral polar amino acid. In some embodiments, the EGFR-binding proteins comprise a light chain domain having an amino acid sequence of SEQ ID NO:59, where positions 3 and 9 are optionally modified to a neutral polar amino acid and position 154 is optionally modified to a negative polar amino acid. In some embodiments, the EGFR-binding proteins comprise a light chain domain having an amino acid sequence of SEQ ID NO:60, where positions 3, 9 and 10 are optionally modified to a neutral polar amino acid and position 154 is optionally modified to a negative polar amino acid.

In some embodiments, the EGFR-binding proteins comprise a heavy chain domain having an amino acid sequence of SEQ ID NO:95, where position 99 is optionally modified to positive polar amino acid. In some embodiments, the EGFR-binding proteins comprise a heavy chain domain having an amino acid sequence of SEQ ID NO:96, where position 114 is optionally modified to positive polar amino acid. In some embodiments, the EGFR-binding proteins comprise a heavy chain domain having an amino acid sequence of SEQ ID NO:55, where position 176 is optionally modified to a neutral polar or a positive polar amino acid.

In some embodiments, the EGFR-binding proteins comprise an Fc domain having an amino acid sequence of SEQ ID NO:61, where position 19 is optionally modified to positive polar amino acid. In some embodiments, the EGFR-binding proteins comprise an Fc domain having an amino acid sequence of SEQ ID NO:62, where position 37 is optionally modified to positive polar amino acid. In some embodiments, the EGFR-binding proteins comprise an Fc domain having an amino acid sequence of SEQ ID NO:63, where position 66 is optionally modified to positive polar amino acid. In some embodiments, the EGFR-binding proteins comprise an Fc domain having an amino acid sequence of SEQ ID NO:64, where position 93 is optionally modified to positive polar amino acid.

In some aspects of the invention, provided herein are EGFR-binding proteins comprising a light chain domain having an amino acid sequence of SEQ ID NO:34 modified to include at least one amino acid substitution selected from L₃Q, L₃K, V₉S, V₉D, I₁₀S, L₁₅₄D, L₁₅₄K; and/or a heavy chain domain having an amino acid sequence of SEQ ID NO:35 modified to include at least one amino acid substitution selected from L₉₉K, L₁₁₄K, L₁₇₆5 and L₁₇₆K. In some embodiments, the EGFR-binding proteins comprise a light chain domain having an amino acid sequence of SEQ ID NO:34 modified to include at least one amino acid substitution selected from L₃K, V₉K, I₁₀K and L₁₅₄K; and/or a heavy chain domain having an amino acid sequence of SEQ ID NO:35 modified to include at least one amino acid substitution selected from L₉₉K, L_{114K} and L₁₇₆K.

In some embodiments, the EGFR-binding proteins comprise a light chain domain having an amino acid sequence of SEQ ID NO:34 modified to include at least two, at least three, or at least four amino acid substitutions selected from L₃Q, L₃K, V₉S, V₉D, I₁₀S, L₁₅₄D and L₁₅₄K. In some embodiments, the EGFR-binding proteins comprise a light chain domain having an amino acid sequence of SEQ ID NO:34 modified to include at least two, at least three, or at least four amino acid substitutions selected from the group consisting of L₃K, V₉K, I₁₀K and L₁₅₄K.

In some embodiments, the EGFR-binding proteins comprises a light chain domain having an amino acid sequence of SEQ ID NO:34 modified to include amino acid substitutions selected from (a) V₉S and I₁₀S, (b) L₃Q and V₉S, (c) L₃Q, V₉S, and I₁₀S (d) L₃, V₉ and L₁₅₄, and (e) L₃Q, V₉S, I₁₀S and L₁₅₄D.

In some embodiments, the EGFR-binding proteins comprise a light chain domain having an amino acid sequence of SEQ ID NO:37, SEQ ID NO:97, SEQ ID NO:38, SEQ ID NO:98, SEQ ID NO:39, SEQ ID NO:40, SEQ ID NO:99, SEQ ID NO:42, SEQ ID NO:43, SEQ ID NO:44, SEQ ID NO:45, SEQ ID NO:46, SEQ ID NO:51, SEQ ID NO:52, SEQ ID NO:53, SEQ ID NO:54, SEQ ID NO:56, SEQ ID NO:57, SEQ ID NO:58, SEQ ID NO:59, or SEQ ID NO:60.

In some embodiments, the EGFR-binding proteins comprise a light chain domain having an amino acid sequence of SEQ ID NO:34 modified to include at least one amino acid substitution selected from the group consisting of a neutral polar amino acid at L₃, a positive polar amino acid at L₃, a neutral polar amino acid at V₉, a negative polar amino acid at V₉, a neutral polar amino acid at I₁₀, a negative polar amino acid at L₁₅₄, and a positive polar amino acid at L₁₅₄, and a heavy chain domain having an amino acid sequence of SEQ ID NO:35. In some embodiments, the neutral polar amino acid at L₃, V₉ and/or I₁₀ of SEQ ID NO:34 may be selected from N, C, Q, H, S, T and Y. In some embodiments, the negative polar amino acid at L₁₅₄ of SEQ ID NO:34 may be D or E.

In some embodiments, the EGFR-binding proteins comprise a light chain domain having an amino acid sequence of SEQ ID NO:34, and a heavy chain domain having an amino acid sequence of SEQ ID NO:35 modified to include at least one amino acid substitution selected from a positive polar amino acid at L₉₉, a positive polar amino acid at L₁₁₄, a neutral polar amino acid at L₁₇₆, and a positive polar amino acid at L₁₇₆. In some embodiments, the neutral polar amino acid at L₁₇₆ of SEQ ID NO:35 may be selected from N, C, Q, H, S, T and Y. In some embodiments, the positive polar amino acid at L₉₉, L₁₁₄ and/or L₁₇₆ may be R or K.

In some embodiments, the EGFR-binding proteins further comprise a human IgG1 Fc domain. In some embodiments, the EGFR-binding proteins further comprise an Fc domain having an amino acid sequence of SEQ ID NO:36. In some embodiments, the Fc domain having the amino acid sequence of SEQ ID NO:36 is modified to include at least one amino acid substitution selected from a positive polar amino acid L₁₉, I₃₇, V₆₆ and L₉₃. In some embodiments, the Fc domain having the amino acid sequence of SEQ ID NO:36 is modified to include at least one amino acid substitution selected from L₁₉K, I₃₇K, V₆₆K and L₉₃K.

In some aspects of the invention, provided herein are EGFR-binding proteins comprising an Fc domain having an amino acid sequence of SEQ ID NO:36 modified to include at least one amino acid substitution selected from a positive polar amino acid at position L₁₉, a positive polar amino acid at position I₃₇, a positive polar amino acid at position V₆₆, and a positive polar amino acid at position L₉₃. In some embodiments, the positive polar amino acid is arginine (R) or lysine (K). In some embodiments, the EGFR-binding proteins comprise an Fc domain having an amino acid sequence of SEQ ID NO:36 modified to include at least one amino acid substitution selected from L₁₉K, I₃₇K, V₆₆K and L₉₃K.

In some embodiments, the EGFR-binding proteins comprise an Fc domain having an amino acid sequence of SEQ ID NO:47, SEQ ID NO:48, SEQ ID NO:49, SEQ ID NO:50, SEQ ID NO:61, SEQ ID NO:62, SEQ ID NO:63 and SEQ ID NO:64.

In some embodiments, the EGFR-binding proteins comprise an Fc domain having an amino acid sequence of SEQ ID NO:36 modified to include at least one amino acid substitution selected from a positive polar amino acid at position L₁₉, a positive polar amino acid at position I₃₇, a positive polar amino acid at position V₆₆, and a positive polar amino acid at position L₉₃; a light chain domain having an amino acid sequence of SEQ ID NO:34; and a heavy chain domain having an amino acid sequence of SEQ ID NO:35.

In some embodiments, the EGFR-binding proteins comprise an Fc domain having an amino acid sequence of SEQ ID NO:36 modified to include at least one amino acid substitution selected from a positive polar amino acid at position L₁₉, a positive polar amino acid at position I₃₇, a positive polar amino acid at position V₆₆, and a positive polar amino acid at position L₉₃; a light chain domain having an amino acid sequence of SEQ ID NO:34 modified to include at least one amino acid substitution selected from a neutral polar amino acid at L₃, a positive polar amino acid at L₃, a neutral polar amino acid at V₉, a negative polar amino acid at V₉, a neutral polar amino acid at I₁₀, and a negative polar amino acid at L₁₅₄, and a positive polar amino acid at L₁₅₄; and a heavy chain domain having an amino acid sequence of SEQ ID NO:35.

In some embodiments, the EGFR-binding proteins comprise an Fc domain having an amino acid sequence of SEQ ID NO:36 modified to include at least one amino acid substitution selected from a positive polar amino acid at position L₁₉, a positive polar amino acid at position I₃₇, a positive polar amino acid at position V₆₆, and a positive polar amino acid at position L₉₃; a light chain domain having an amino acid sequence of SEQ ID NO:34; and heavy chain domain having an amino acid sequence of SEQ ID NO:35 modified to include at least one amino acid substitution selected from a positive polar amino acid at L₉₉, a positive polar amino acid at L₁₁₄, a neutral polar amino acid at L₁₇₆, and a positive polar amino acid at L₁₇₆.

In some embodiments, the EGFR-binding proteins are in the form of monoclonal antibodies such as, for example, chimeric monoclonal antibodies or humanized monoclonal antibodies. In some embodiments, the EGFR-binding proteins are in the form of antigen-binding antibody fragments such as, for example, Fab antibody fragments. In some embodiments, the EGFR-binding proteins are in the form of antibody-like proteins such as, for example, fusion proteins, single-chain Fv antibody fragments, and/or minibodies. In some embodiments, the EGFR-binding proteins are conjugated to a toxin and/or a radioisotope.

In some embodiments, the EGFR-binding proteins are lyophilized. In some embodiments, the EGFR-binding proteins are in solution.

In some aspects of the invention, provided herein are compositions comprising any of the EGFR-binding proteins of the invention. In some embodiments, the compositions comprise EGFR-binding proteins comprising a light chain domain having an amino acid sequence of SEQ ID NO:34 modified to include at least one amino acid substitution selected from a neutral polar amino acid at L₃, a positive polar amino acid at L₃, a neutral polar amino acid at V₉, a negative polar amino acid at V₉, a neutral polar amino acid at I₁₀, a negative polar amino acid at L₁₅₄, and a positive polar amino acid at L₁₅₄; and/or a heavy chain domain having an amino acid sequence of SEQ ID NO:35 modified to include at least one amino acid substitution selected from a positive polar amino acid at L₉₉, a positive polar amino acid at L₁₁₄, a neutral polar amino acid at L₁₇₆, and a positive polar amino acid at L₁₇₆.

In some embodiments, the compositions comprise EGFR-binding proteins comprising a light chain domain having an amino acid sequence of SEQ ID NO:34 modified to include at least one amino acid substitution selected from L₃Q, L₃K, V₉S, V₉D, I₁₀S, L₁₅₄D and L₁₅₄K; and/or a heavy chain domain having an amino acid sequence of SEQ ID NO:35 modified to include at least one amino acid substitution selected from L₉₉K, L₁₁₄K, L₁₇₆5 and L₁₇₆K.

In some embodiments, the compositions comprise EGFR-binding proteins comprising an Fc domain having an amino acid sequence of SEQ ID NO:36 modified to include at least one amino acid substitution selected from a positive polar amino acid at position L₁₉, a positive polar amino acid at position I₃₇, a positive polar amino acid at position V₆₆, and a positive polar amino acid at position L₉₃. In some embodiments, the compositions comprise EGFR-binding proteins comprising an Fc domain having an amino acid sequence of SEQ ID NO:36 modified to include at least one amino acid substitution selected from L₁₉K, I₃₇K, V₆₆K and L₉₃K.

In some embodiments, the compositions comprise the EGFR-binding proteins at a concentration of about 20 mg/ml to about 350 mg/ml. In some embodiments, the concentration of the EGFR-binding proteins is about 100 mg/ml to about 250 mg/ml. In some embodiments, the concentration of the EGFR-binding proteins is about 120 mg/ml to about 150 mg/ml. In some embodiments, the concentration of the EGFR-binding proteins is about 120 mg/ml, about 130 mg/ml or about 140 mg/ml. In some embodiments, the concentration of the EGFR-binding proteins is about 120 mg/ml or is 120 mg/ml.

In some embodiments, the compositions are liquid. In some embodiments, the compositions are formulated for subcutaneous injection and/or other non-intravenous delivery routes such as, for example, intramuscular injection.

In some embodiments, the compositions further comprise at least one buffer, at least one stabilizer and/or at least one surfactant. In some embodiments, the buffer is present at a concentration of about 1 mM to about 100 mM. In some embodiments, the buffer provides a pH of 5.5±2.0. In some embodiments, the stabilizer is present at a concentration of about 100 mM to about 500 mM. In some embodiments, a secondary stabilizer is present. In some embodiments, the secondary stabilizer is arginine, ornithine, lysine, histidine, glutamic acid, aspartic acid, isoleucine, leucine, alanine, phenylalanine, tyrosine, tryptophan, methionine, serine or proline (e.g., as a hydrochloride). In some embodiments, methionine is present at a concentration of about 5 mM to about 25 mM. In some embodiments, the surfactant is a nonionic surfactant. In some embodiments, the surfactant is present at a concentration of about 0.01% to about 0.1%. In some embodiments, the compositions further comprise at least one pharmaceutically acceptable carrier, excipient, and/or diluent. In some embodiments, the compositions further comprise additional agents (e.g., therapeutic agents). In some embodiments, the compositions are sterile.

In some embodiments, the compositions comprise the EGFR-binding proteins, histidine HCl, trehalose dehydrate, methionine and/or polysorbate 80. In some embodiments, the compositions comprise about 100 mg/ml to about 150 mg/ml of the EGFR-binding proteins, about 10 mM to about 30 mM histidine HCl, about 200 mM to about 220 mM trehalose dehydrate, about 5 mM to about 15 mM methionine and/or about 0.04% to about 0.08% polysorbate 80. In some embodiments, the compositions comprise about 120 mg/ml of the EGFR-binding proteins, about 20 mM histidine HCl, about 210 mM trehalose dehydrate, about 10 mM methionine and/or about 0.06% polysorbate 80.

In some aspects of the invention, provided herein are methods of treating a condition in a subject in need thereof, comprising administering to the subject any of the EGFR-binding proteins and/or compositions described herein. In some embodiments, the methods comprise administering to the subject a therapeutically effective amount of any of the EGFR-binding proteins and/or compositions provided herein. In some embodiments, the condition is cancer or a non-malignant condition. In some embodiments, the condition involves EGFR-expressing cells. In some embodiments, the cancer is a human cancer such as, for example, brain tumor, tumor of the urogenital tract, tumor of the lymphatic system, stomach tumor, laryngeal tumor, monocytic leukemia, lung adenocarcinoma, small-cell lung carcinoma, pancreatic cancer, glioblastoma, breast carcinoma, and/or other cancers expressing or overexpressing EGFR. In some embodiments, the non-malignant condition is a non-malignant tumor.

In some aspects of the invention, provided herein are uses of the EGFR-binding proteins for the preparation of a medicament for treating a condition amenable to treatment with an EGFR-binding protein.

In some aspects of the invention, provided herein are nucleic acids encoding the EGFR-binding proteins, vectors comprising the nucleic acids, expression cassettes comprising the nucleic acids, and host cells comprising the nucleic acids and/or vectors and/or expression cassettes. In some embodiments, the nucleic acids are isolated.

In some aspects of the invention, provided herein are methods of producing an EGFR-binding protein (e.g., an anti-EGFR antibody), the methods comprising culturing any of the host cells described herein to produce the EGFR-binding protein. In certain embodiments, the methods include an additional step of isolating the EGFR-binding protein.

In some aspects of the invention, provided herein are kits comprising a container and any of the EGFR-binding proteins and/or compositions contained therein. In some embodiments, the kits further comprise instructions for using the kits. In some embodiments, the kits further comprise a package insert or label indicating that the kits can be used to treat cancer or a non-malignant condition, for example, one characterized by the overexpression of EGFR. In some embodiments, the kits comprise injection components such as, for example, a syringe or a syringe filled with the EGFR-binding proteins or a composition containing the EGFR-binding proteins. In some embodiments, the volume of the composition in the kits is about 2 ml. In some embodiments, the volume of the composition is less than 2 ml. In some embodiments, the concentration of the EGFR-binding proteins in about 2 ml or less of a composition is about 100 mg/ml to about 150 mg/ml. In some embodiments, the concentration of the EGFR-binding proteins is about 120 mg/ml or is 120 mg/ml.

These and other aspects and embodiments of the invention will be described in greater detail herein.

### BRIEF DESCRIPTION OF DRAWINGS

FIG. 1 is a graph showing monomer loss for select CD20-binding proteins of the invention. Aggregation conditions were 10 mg/ml protein, 20 mM histidine HCl, pH 6.5, 60° C. Monomer concentration was monitored using size exclusion high-performance liquid chromatography (HPLC).

FIG. 2 is a graph showing monomer loss for select CD20-binding proteins of the invention. Aggregation conditions were 20 mg/ml protein, 20 mM histidine HCl, pH 6.5, 58° C. Monomer concentration was monitored using size exclusion HPLC.

FIG. 3 is a graph showing monomer loss for select CD20-binding proteins of the invention. Aggregation conditions were 20 mg/ml protein, 20 mM histidine HCl, pH 6.5, 58° C. Monomer concentration was monitored using size exclusion HPLC.

FIG. 4 is a graph showing results from repeat aggregation experiments for select CD20-binding proteins of the invention, demonstrating reproducibility. Aggregation conditions were 20 mg/ml protein, 20 mM histidine HCl, pH 6.5, 58° C. Monomer concentration was monitored using size exclusion HPLC.

FIG. 5 is a graph showing monomer loss for select CD20-binding proteins of the invention. Aggregation conditions were 20 mg/ml protein, 20 mM histidine HCl, pH 6.5, 58° C. Monomer concentration was monitored using size exclusion HPLC.

FIG. 6 is a graph showing raw and analyzed data from an immunofluorescence assay showing total binding, specific binding and nonspecific binding of “wild type” (unmodified) rituximab (trade name RITUXAN®) using the Raji cell line.

FIG. 7 is a graph of raw and analyzed data from an immunofluorescence assay in a linear plot.

FIG. 8 is a graph of immunofluorescence assay results showing specific binding of select CD20-binding proteins of the invention using the Raji cell line. Results showed that control antibody (rituximab) and the select CD20-binding proteins having stabilizing mutations outside of the complementarity determining region (CDR) (e.g., M1/M2/M3/M4/M7) bind to the target antigen with the same level of affinity as published values. The mutation in the CDR (M9) diminishes binding.

### BRIEF DESCRIPTION OF SEQUENCE LISTING

SEQ ID NO:1 is the amino acid sequence of a light chain domain of rituximab.

SEQ ID NO:2 is the amino acid sequence of an Fab heavy chain domain of rituximab.

SEQ ID NO:3 is the amino acid sequence of an Fc domain of rituximab.

SEQ ID NO:4 is the amino acid sequence of a light chain domain of rituximab modified at position V₃Q.

SEQ ID NO:5 is the amino acid sequence of a light chain domain of rituximab modified at position A₉S.

SEQ ID NO:6 is the amino acid sequence of a light chain domain of rituximab modified at position I₁₀S.

SEQ ID NO:7 is the amino acid sequence of a light chain domain of rituximab modified at position V₅₉S.

SEQ ID NO:8 is the amino acid sequence of a light chain domain of rituximab modified at position L₁₅₃D.

SEQ ID NO:9 is the amino acid sequence of an Fab heavy chain domain of rituximab modified at position L₁₇₈S.

SEQ ID NO:10 is the amino acid sequence of a light chain domain of rituximab modified at positions A₉S and I₁₀S.

SEQ ID NO:11 is the amino acid sequence of a light chain domain of rituximab modified at positions V₃Q, A₉S and V₅₉S.

SEQ ID NO:12 is the amino acid sequence of a light chain domain of rituximab modified at positions V₃Q, A₉S, I₁₀S and V₅₉S.

SEQ ID NO:13 is the amino acid sequence of a light chain domain of rituximab modified at positions V₃Q, A₉S, V₅₉S and L₁₅₃D.

SEQ ID NO:14 is the amino acid sequence of a light chain domain of rituximab modified at positions V₃Q, A₉S, I₁₀S, V₅₉S and L₁₅₃D.

SEQ ID NO:15 is the amino acid sequence of an Fc domain of rituximab modified at position L₁₅K.

SEQ ID NO:16 is the amino acid sequence of an Fc domain of rituximab modified at position I₃₃K.

SEQ ID NO:17 is the amino acid sequence of an Fc domain of rituximab modified at position V₆₂K.

SEQ ID NO:18 is the amino acid sequence of an Fc domain of rituximab modified at position L₈₉K.

SEQ ID NO:19 is the amino acid sequence of a light chain domain of rituximab modified at position V₃.

SEQ ID NO:20 is the amino acid sequence of a light chain domain of rituximab modified at position A₉.

SEQ ID NO:21 is the amino acid sequence of a light chain domain of rituximab modified at position I₁₀.

SEQ ID NO:22 is the amino acid sequence of a light chain domain of rituximab modified at position V₅₉.

SEQ ID NO:23 is the amino acid sequence of a light chain domain of rituximab modified at position L₁₅₃.

SEQ ID NO:24 is the amino acid sequence of an Fab heavy chain domain of rituximab modified at position L₁₇₈.

SEQ ID NO:25 is the amino acid sequence of a light chain domain of rituximab modified at positions A₉ and I₁₀.

SEQ ID NO:26 is the amino acid sequence of a light chain domain of rituximab modified at positions V₃, A₉ and V₅₉.

SEQ ID NO:27 is the amino acid sequence of a light chain domain of rituximab modified at positions V₃, A₉, I₁₀ and V₅₉.

SEQ ID NO:28 is the amino acid sequence of a light chain domain of rituximab modified at positions V₃, A₉, V₅₉ and L₁₅₃.

SEQ ID NO:29 is the amino acid sequence of a light chain domain of rituximab modified at positions V₃, A₉, I₁₀, V₅₉ and L₁₅₃.

SEQ ID NO:30 is the amino acid sequence of an Fc domain of rituximab modified at position L₁₅.

SEQ ID NO:31 is the amino acid sequence of an Fc domain of rituximab modified at position I₃₃.

SEQ ID NO: 32 is the amino acid sequence of an Fc domain of rituximab modified at position V₆₂.

SEQ ID NO:33 is the amino acid sequence of an Fc domain of rituximab modified at position L₈₉.

SEQ ID NO:34 is the amino acid sequence of a light chain domain of cetuximab.

SEQ ID NO:35 is the amino acid sequence of an Fab heavy chain domain of cetuximab.

SEQ ID NO:36 is the amino acid sequence of an Fc domain of cetuximab.

SEQ ID NO:37 is the amino acid sequence of a light chain domain of cetuximab modified at position L₃Q.

SEQ ID NO:38 is the amino acid sequence of a light chain domain of cetuximab modified at position V₉S.

SEQ ID NO:39 is the amino acid sequence of a light chain domain of cetuximab modified at position I₁₀S.

SEQ ID NO:40 is the amino acid sequence of a light chain domain of cetuximab modified at position L₁₅₄D.

SEQ ID NO:41 is the amino acid sequence of an Fab heavy chain domain of cetuximab modified at position L₁₇₆S.

SEQ ID NO:42 is the amino acid sequence of a light chain domain of cetuximab modified at positions V₉S and I₁₀S.

SEQ ID NO:43 is the amino acid sequence of a light chain domain of cetuximab modified at positions L₃Q and V₉S.

SEQ ID NO:44 is the amino acid sequence of a light chain domain of cetuximab modified at positions L₃Q, V₉S and I₁₀S.

SEQ ID NO:45 is the amino acid sequence of a light chain domain of cetuximab modified at positions L₃Q, V₉S and L₁₅₄D.

SEQ ID NO:46 is the amino acid sequence of a light chain domain of cetuximab modified at positions L₃Q, V₉S, I₁₀S and L₁₅₄D.

SEQ ID NO:47 is the amino acid sequence of an Fc domain of cetuximab modified at position L₁₉K.

SEQ ID NO:48 is the amino acid sequence of an Fc domain of cetuximab modified at position I₃₇K.

SEQ ID NO:49 is the amino acid sequence of an Fc domain of cetuximab modified at position V₆₆K.

SEQ ID NO:50 is the amino acid sequence of an Fc domain of cetuximab modified at position L₉₃K.

SEQ ID NO:51 is the amino acid sequence of a light chain domain of cetuximab modified at position L₃.

SEQ ID NO:52 is the amino acid sequence of a light chain domain of cetuximab modified at position V₉.

SEQ ID NO:53 is the amino acid sequence of a light chain domain of cetuximab modified at position I₁₀.

SEQ ID NO:54 is the amino acid sequence of a light chain domain of cetuximab modified at position L₁₅₄.

SEQ ID NO:55 is the amino acid sequence of an Fab heavy chain domain of cetuximab modified at position L₁₇₆.

SEQ ID NO:56 is the amino acid sequence of a light chain domain of cetuximab modified at positions V₉ and I₁₀.

SEQ ID NO:57 is the amino acid sequence of a light chain domain of cetuximab modified at positions L₃ and V₉.

SEQ ID NO:58 is the amino acid sequence of a light chain domain of cetuximab modified at positions L₃, V₉ and I₁₀.

SEQ ID NO:59 is the amino acid sequence of a light chain domain of cetuximab modified at positions L₃, V₉ and L₁₅₄.

SEQ ID NO:60 is the amino acid sequence of a light chain domain of cetuximab modified at positions L₃, V₉, I₁₀ and L₁₅₄.

SEQ ID NO:61 is the amino acid sequence of an Fc domain of cetuximab modified at position L₁₉.

SEQ ID NO:62 is the amino acid sequence of an Fc domain of cetuximab modified at position I₃₇.

SEQ ID NO:63 is the amino acid sequence of an Fc domain of cetuximab modified at position V₆₆.

SEQ ID NO:64 is the amino acid sequence of an Fc domain of cetuximab modified at position L₉₃.

SEQ ID NO:65 is the amino acid sequence of human B-lymphocyte antigen CD20 (GenBank Protein ID CAA31046.1).

SEQ ID NO:66 is the amino acid sequence of human epidermal growth factor receptor (GenBank Protein ID CAA25240.1).

SEQ ID NO:67 is the nucleotide sequence of a light chain domain of rituximab, including the restrictions sites (underlined and italicized) used for subcloning into the GWIZ expression vector. The underlined sequence represents the leader sequence that includes the start codon and is removed during posttranslational modification. The italicized sequences represent the codons for targeted mutations.

SEQ ID NO:68 is the forward primer oligonucleotide sequence used to introduce the V₃Q mutation in the light chain domain of rituximab. The codon containing the mutation is italicized.

SEQ ID NO:69 is the forward primer oligonucleotide sequence used to introduce the A₉S mutation in the light chain domain of rituximab. The codon containing the mutation is italicized.

SEQ ID NO:70 is the forward primer oligonucleotide sequence used to introduce the I₁₀S mutation in the light chain domain of rituximab. The codon containing the mutation is italicized.

SEQ ID NO:71 is the forward primer oligonucleotide sequence used to introduce the V₅₉S mutation in the light chain domain of rituximab. The codon containing the mutation is italicized.

SEQ ID NO:72 is the forward primer oligonucleotide sequence used to introduce the L₁₅₃D mutation in the light chain domain of rituximab. The codon containing the mutation is italicized.

SEQ ID NO:73 is the oligonucleotide nucleotide sequence of a Fab heavy chain domain of rituximab, including the restrictions sites (underlined and italicized) used for subcloning into the GWIZ expression vector. The underlined sequence represents the leader sequence that includes the start codon and is removed during posttranslational modification. The italicized sequences represent the codons for targeted mutations.

SEQ ID NO:74 is the forward primer oligonucleotide sequence used to introduce the L₁₇₈S mutation in the Fab heavy chain domain of rituximab. The codon containing the mutation is italicized.

SEQ ID NO:75 is the forward primer oligonucleotide sequence used to introduce the Y₁₀₁S mutation in the Fab heavy chain domain of rituximab. The codon containing the mutation is italicized.

SEQ ID NO:76 is the nucleotide sequence of a light chain domain of cetuximab, including the restrictions sites (underlined and italicized) used for subcloning into the GWIZ expression vector. The underlined sequence represents the leader sequence that includes the start codon and is removed during posttranslational modification. The italicized sequences represent the codons for targeted mutations.

SEQ ID NO:77 is the forward primer oligonucleotide sequence used to introduce the L₃Q mutation in the light chain domain of cetuximab. The codon containing the mutation is italicized.

SEQ ID NO:78 is the reverse primer oligonucleotide sequence used to introduce the L₃Q mutation in the light chain domain of cetuximab. The codon containing the mutation is italicized.

SEQ ID NO:79 is the forward primer oligonucleotide sequence used to introduce the V₉S mutation in the light chain domain of cetuximab. The codon containing the mutation is italicized.

SEQ ID NO:80 is the reverse primer oligonucleotide sequence used to introduce the V₉S mutation in the light chain domain of cetuximab. The codon containing the mutation is italicized.

SEQ ID NO:81 is the forward primer oligonucleotide sequence used to introduce the I₁₀S mutation in the light chain domain of cetuximab. The codon containing the mutation is italicized.

SEQ ID NO:82 is the reverse primer oligonucleotide sequence used to introduce the I_{R}S mutation in the light chain domain of cetuximab. The codon containing the mutation is italicized.

SEQ ID NO:83 is the forward primer oligonucleotide sequence used to introduce the V₉S and I₁₀S mutations in the light chain domain of cetuximab. The codons containing the mutations are italicized.

SEQ ID NO:84 is the reverse primer oligonucleotide sequence used to introduce the V₉S and I₁₀S mutations in the light chain domain of cetuximab. The codons containing the mutations are italicized.

SEQ ID NO:85 is the forward primer oligonucleotide sequence used to introduce the L₁₅₄D mutation in the light chain domain of cetuximab. The codon containing the mutation is italicized.

SEQ ID NO:86 is the reverse primer oligonucleotide sequence used to introduce the L₁₅₄D mutation in the light chain domain of cetuximab. The codon containing the mutation is italicized.

SEQ ID NO:87 is the nucleotide sequence of a Fab heavy chain domain of cetuximab, including the restrictions sites (underlined and italicized) used for subcloning into the GWIZ expression vector. The underlined sequence represents the leader sequence that includes the start codon and is removed during posttranslational modification.

SEQ ID NO:88 is the amino acid sequence of an Fab heavy chain domain of rituximab modified at position Y₁₀₁.

SEQ ID NO:89 is the amino acid sequence of an Fab heavy chain domain of rituximab modified at position Y₁₀₁H.

SEQ ID NO:90 is the amino acid sequence of a light chain domain of rituximab modified at position V₁₀₉.

SEQ ID NO:91 is the amino acid sequence of a light chain domain of rituximab modified at position I₁₀K.

SEQ ID NO:92 is the amino acid sequence of a light chain domain of rituximab modified at position V₅₉D.

SEQ ID NO:93 is the amino acid sequence of a light chain domain of rituximab modified at position V₁₀₉K.

SEQ ID NO:94 is the amino acid sequence of an Fab heavy chain domain of rituximab modified at position L₁₇₈K.

SEQ ID NO:95 is the amino acid sequence of an Fab heavy chain domain of cetuximab modified at position L₉₉.

SEQ ID NO:96 is the amino acid sequence of an Fab heavy chain domain of cetuximab modified at position L₁₁₄.

SEQ ID NO:97 is the amino acid sequence of a light chain domain of cetuximab modified at position L₃K.

SEQ ID NO:98 is the amino acid sequence of a light chain domain of cetuximab modified at position V₉D.

SEQ ID NO:99 is the amino acid sequence of a light chain domain of cetuximab modified at position L₁₅₄K.

SEQ ID NO:100 is the amino acid sequence of an Fab heavy chain domain of cetuximab modified at position L₉₉K.

SEQ ID NO:101 is the amino acid sequence of an Fab heavy chain domain of cetuximab modified at position L₁₁₄K.

SEQ ID NO:102 is the amino acid sequence of an Fab heavy chain domain of cetuximab modified at position L₁₇₆K.

### DETAILED DESCRIPTION OF INVENTION

Compositions containing proteins (e.g., monoclonal antibodies) are, in many instances, injected or infused via the intravenous route of administration. There is a desire to provide compositions for subcutaneous injection, which can be administered outside of the clinical setting and without a medical practitioner's assistance. Viscoelastic resistance to hydraulic conductance in the subcutaneous tissue, backpressure generated upon injection, and perceptions of pain all limit subcutaneous injection volumes to approximately 2 ml. Therefore, protein compositions for subcutaneous injection must contain highly concentrated, stable proteins. The preparation of highly concentrated protein compositions is challenging because, at high concentrations, many proteins begin to aggregate causing protein degradation and, in some cases, immunogenicity of the protein. Immunogenic reaction against aggregates of protein may lead to the production of neutralizing antibodies, which can render the protein ineffective over time.

The invention provides, inter alia, CD20-binding proteins and epidermal growth factor receptor (EGFR)-binding proteins that have a reduced tendency to aggregate (e.g., are more stable) as compared to existing proteins. The invention also provides compositions comprising the proteins and methods of use.

The exposure of hydrophobic residues to an aqueous solvent is a thermodynamically unfavorable condition and processes such as aggregation, which minimize exposure of these residues, are favored (Roberts, C. J. J. Phys. Chem., 2003; 107(5): 1194-1207). This aggregation leads to protein degradation. The present invention is premised, in part, on the identification of CD20-binding proteins and EGFR-binding proteins having a reduced tendency to aggregate, for example, when present in compositions (e.g., liquid composition). Surprisingly, prior to the invention, the inventors found that they were not able to predict what amino acid modifications or what combinations of amino acid modifications reduce the tendency of CD20-binding proteins and EGFR-binding proteins to aggregate. A simple amino acid substitution in an existing protein from a hydrophobic residue to any hydrophilic residue did not necessarily reduce the tendency of the protein to aggregate. Rather, the inventors found that myriad factors affect protein aggregation, including the number of amino acid substitutions in the protein, the location of such substitutions, side-chain polarity, side-chain charge, and proximity of side chains relative to one another, and that only certain substitutions actually resulted in reduced aggregation.

### Proteins

The invention provides, inter alia, CD20-binding proteins and EGFR-binding proteins that are less prone to aggregation as compared to existing proteins.

### CD20-Binding Proteins

In some aspects of the invention, provided herein are proteins that selectively bind to B lymphocyte antigen CD20 (i.e., CD20 antigen) and are referred to as “CD20-binding proteins.” A protein (e.g., anti-CD20 antibody) selectively binds to an antigen (e.g., CD20) if the protein binds or is capable of binding to the antigen with a greater affinity than the affinity with which the protein might bind to other proteins (e.g., proteins other than CD20). Such binding may be measured or determined by standard protein-protein interaction assays (e.g., antibody-antigen or ligand-receptor assays) such as, for example, competitive assays, saturation assays, or standard immunoassays including, without limitation, enzyme-linked immunosorbent assays, radioimmunoassays and radio-immuno-filter binding assays.

CD20 protein is a non-glycosylated, transmembrane phosphoprotein with a molecular weight of approximately 35 kD. It is found on the surface of greater than 90% of B cells from peripheral blood and/or lymphoid organs and is present on both normal B cells and malignant B cells. CD20 is expressed during early pre-B cell development and remains expressed until plasma cell differentiation. In some embodiments, the CD20 antigen may be human CD20 antigen. An example of an amino acid sequence of a human CD20 antigen is represented as GenBank Protein ID CAA31046.1 (SEQ ID NO:65).

In some embodiments, the CD20-binding proteins of the invention may comprise a light chain domain having an amino acid sequence of SEQ ID NO:1, or a light chain domain having an amino acid sequence of SEQ ID NO:1 modified to include at least one amino acid substitution at the valine at position 3 (V₃), the alanine at position 9 (A₉), the isoleucine at position 10 (I₁₀), the valine at position 59 (V₅₉), the valine at position 109 (V₁₀₉) and/or the leucine at position 153 (L₁₅₃). Valine, alanine, isoleucine and leucine are neutral nonpolar amino acids. In some embodiments, the invention contemplates substituting neutral nonpolar amino acids with neutral polar amino acids, negative polar amino acids, or positive polar amino acids. In some embodiments, V₃, A₉, I₁₀ and/or V₅₉ may be substituted for a neutral polar amino. In some embodiments, V₅₉ and/or L₁₅₃ may be substituted for a negative polar amino acid. In some embodiments, I₁₀ and/or V₁₀₉ may be substituted for a positive polar amino acid.

The amino acids, used according to the invention, may be naturally occurring or synthetic. Naturally occurring neutral polar amino acids include asparagine (N), cysteine (C), glutamine (Q), histidine (H), serine (S), threonine (T), and tyrosine (Y). Naturally occurring positively charged polar amino acids (i.e., positive polar amino acids) include arginine (R) and lysine (K). Naturally occurring negatively charged polar amino acids (i.e., negative polar amino acids) include aspartic acid (D) and glutamic acid (E).

At least one amino acid substitution in the sequence of SEQ ID NO:1 may be selected from: V₃Q (valine to glutamine at position 3), A₉S (alanine to serine at position 9), I₁₀S (isoleucine to serine at position 10), I₁₀K (isoleucine to lysine at position 10), V₅₉5 (valine to serine at position 59), V₅₉D (valine to aspartic acid at position 59), V₁₀₉K (valine to lysine at position 109), and L₁₅₃D (leucine to aspartic acid at position 153). In some embodiments, the CD20-binding proteins may have at least two, at least three, at least four, at least five, or at least six amino acid substitutions. In some embodiments, the CD20-binding proteins may comprise a light chain domain having an amino acid sequence of SEQ ID NO:1 modified to include amino acid substitutions at A₉ and I₁₀. The amino acid substitutions may be A₉S and I₁₀S. In other embodiments, the CD20-binding proteins may comprise a light chain domain having an amino acid sequence of SEQ ID NO:1 modified to include amino acid substitutions at V₃, A₉ and V₅₉. The amino acid substitutions may be V₃Q, A₉S and V₅₉S. In yet other embodiments, the CD20-binding proteins may comprise a light chain domain having an amino acid sequence of SEQ ID NO:1 modified to include amino acid substitutions at V₃, A₉, I₁₀ and V₅₉. The amino acid substitutions may be V₃Q, A₉S, I₁₀S and V₅₉S. In still other embodiments, the CD20-binding proteins may comprise a light chain domain having an amino acid sequence of SEQ ID NO:1 modified to include amino acid substitutions at V₃, A₉, V₅₉ and L₁₅₃. The amino acid substitutions may be V₃Q, A₉S, V₅₉5 and L₁₅₃D. In further embodiments, the CD20-binding proteins may comprise a light chain domain having an amino acid sequence of SEQ ID NO:1 modified to include amino acid substitutions at V₃, A₉, I₁₀, V₅₉ and L₁₅₃. The amino acid substitutions may be V₃Q, A₉S, I₁₀S, V₅₉5 and L₁₅₃D.

In some embodiments, the CD20-binding proteins may comprise a heavy chain domain having an amino acid sequence of SEQ ID NO:2, or a heavy chain domain having an amino acid sequence of SEQ ID NO:2 modified to include at least one amino acid substitution at a position selected from the tyrosine at position 101 (Y₁₀₁) and the leucine at position 178 (L₁₇₈). In some embodiments, L₁₇₈ may be substituted with a neutral polar amino acid. In some embodiments, the neutral polar amino acid may be serine. Thus, in some embodiments, the amino acid substitution may be L₁₇₈S. In some embodiments, L₁₇₈ may be substituted with a positive polar amino acid. In some embodiments, the positive polar amino acid may be lysine. Thus, in some embodiments, the amino acid substitution may be L₁₇₈K. In some embodiments, Y₁₀₁ may be substituted with a neutral polar amino acid. In some embodiments, the neutral polar amino acid may be histidine. Thus, in some embodiments, the amino acid substitution may be Y₁₀₁H.

In some embodiments, the CD20-binding proteins may comprise a human IgG Fc domain. In some embodiments, the CD20-binding proteins may comprise an Fc domain having an amino acid sequence of SEQ ID NO:3, or an Fc domain having an amino acid sequence of SEQ ID NO:3 modified to include at least one amino acid substitution at the leucine at position 15 (L₁₅), the isoleucine at position 33 (I₃₃), the valine at position 62 (V₆₂), and/or the leucine at position 89 (L₈₉). At least one amino acid substitution may be selected from L₁₅K, I₃₃K, V₆₂K and L₈₉K.

In some embodiments, the CD20-binding proteins may comprise any of the modified light chain domains described herein (e.g., SEQ ID NOs. 4-8, 10-14, 19-23, 25-29, 90-93) or a light chain domain having an amino acid sequence of SEQ ID NO:1; and/or any of the modified heavy chain domains described herein (e.g., SEQ ID NOs. 9, 24, 88, 89, 94) or a heavy chain domain having an amino acid sequence of SEQ ID NO:2; and/or a human IgG1 Fc domain, any of the modified Fc domains described herein (e.g., SEQ ID NOs. 15-18 and 30-33), or an Fc domain having an amino acid sequence of SEQ ID NO:3.

In certain embodiments, the neutral nonpolar amino acid at V₃, A₉, I₁₀, V₅₉, V₁₀₉ and/or L₁₅₃ of SEQ ID NO:1, and/or at Y₁₀₁ and/or L₁₇₈ of SEQ ID NO:2, and/or at L₁₅, I₃₃, V₆₂ and/or L₈₉ of SEQ ID NO:3 may be substituted for a charged (e.g., positive or negative) or neutral polar amino acid. In some embodiments, the amino acid substitution at L₁₅₃ of SEQ ID NO:1 is not lysine (K). In some embodiments, the amino acid substitution at L₁₇₈ of SEQ ID NO:2 is not K.

EGFR-Binding Proteins

In other aspects of the invention, provided herein are proteins that may selectively bind to epidermal growth factor receptor (EGFR) and are referred to as “EGFR-binding proteins.” The EGFR is a transmembrane glycoprotein that binds to epidermal growth factor and has a molecular weight of approximately 135 kD. Binding of the EGFR to a ligand induces receptor dimerization and tyrosine autophosphorylation, which leads to cell proliferation. In some embodiments, the EGFR may be human EGFR. An example of an amino acid sequence of a human EGFR is represented as GenBank Protein ID CAA25240.1 (SEQ ID NO:66).

In some embodiments, the EGFR-binding proteins of the invention may comprise a light chain domain having an amino acid sequence of SEQ ID NO:34, or a light chain domain having an amino acid sequence of SEQ ID NO:34 modified to include at least one amino acid substitution at the leucine at position 3 (L₃), the valine at position 9 (V₉), the isoleucine at position 10 (I₁₀), and/or the leucine at position 154 (L₁₅₄). Leucine, valine and isoleucine are neutral nonpolar amino acids. In some embodiments, the invention contemplates substituting neutral nonpolar amino acids with neutral polar amino acids, negative polar amino acids, or positive polar amino acids. In some embodiments, L₃, V₉ and/or I₁₀ may be substituted for a neutral polar amino. In some embodiments, V₉ and/or L₁₅₄ may be substituted for a negative polar amino acid. In some embodiments, L₃ and/or L₁₅₄ may be substituted for a positive polar amino acid.

At least one amino acid substitution in the sequence of SEQ ID NO:34 may be selected from L₃Q (leucine to glutamine at position 3), L₃K (leucine to lysine at position 3), V₉S (valine to serine at position 9), V₉D (valine to aspartic acid at position 9), I₁₀S (isoleucine to serine at position 10), L₁₅₄K (leucine to lysine at position 154), and L₁₅₄D (leucine to aspartic acid at position 154). In some embodiments, the EGFR-binding proteins may have at least two, at least three, at least four, or at least five amino acid substitutions. In some embodiments, the EGFR-binding proteins may comprise a light chain domain having an amino acid sequence of SEQ ID NO:34 modified to include amino acid substitutions at V₉ and I₁₀. The amino acid substitutions may be V₉S and I₁₀S. In other embodiments, the EGFR-binding proteins may comprise a light chain domain having an amino acid sequence of SEQ ID NO:34 modified to include amino acid substitutions at L₃ and V₉. The amino acid substitutions may be L₃Q and V₉S. In yet other embodiments, the EGFR-binding proteins may comprise a light chain domain having an amino acid sequence of SEQ ID NO:34 modified to include amino acid substitutions at L₃, V₉, and I₁₀. The amino acid substitutions may be L₃Q, V₉S, and I₁₀S. In still other embodiments, the EGFR-binding proteins may comprise a light chain domain having an amino acid sequence of SEQ ID NO:34 modified to include amino acid substitutions at A₃, V₉, and L₁₅₄. The amino acid substitutions may be L₃Q, V₉S, and L₁₅₄D. In further embodiments, the EGFR-binding proteins may comprise a light chain domain having an amino acid sequence of SEQ ID NO:34 modified to include amino acid substitutions at L₃, V₉, I₁₀ and L₁₅₄. The amino acid substitutions may be L₃Q, V₉S, I₁₀S and L₁₅₄D.

In some embodiments, the EGFR-binding proteins may comprise a heavy chain domain having an amino acid sequence of SEQ ID NO:35, or a heavy chain domain having an amino acid sequence of SEQ ID NO:35 modified to include at least one amino acid substitution selected from the leucine at position 99 (L₉₉), the leucine at position 114 (L₁₁₄), and the leucine at position 176 (L₁₇₆). In some embodiments, L₉₉ may be substituted with a positive polar amino acid. In some embodiments, the positive polar amino acid may be lysine. Thus, in some embodiments, the amino acid substitution may be L₉₉K. In some embodiments, L₁₁₄ may be substituted with a positive polar amino acid. In some embodiments, the positive polar amino acid may be lysine. Thus, in some embodiments, the amino acid substitution may be L₁₁₄K. In some embodiments, L₁₇₆ may be substituted with a neutral polar amino acid. In some embodiments, the neutral polar amino acid may be serine. Thus, in some embodiments, the amino acid substitution may be L₁₇₆S. In some embodiments, L₁₇₆ may be substituted with a positive polar amino acid. In some embodiments, the positive polar amino acid may be lysine. Thus, in some embodiments, the amino acid substitution may be L₁₇₆K.

In some embodiments, the EGFR-binding proteins may comprise a human IgG Fc domain. In some embodiments, the EGFR-binding proteins may comprise an Fc domain having an amino acid sequence of SEQ ID NO:36, or an Fc domain having an amino acid sequence of SEQ ID NO:36 modified to include at least one amino acid substitution at the leucine at position 19 (L₁₉), at the isoleucine at position 37 (I₃₇), the valine at position 66 (V₆₆), and/or the leucine at position 93 (L₉₃).

In some embodiments, the EGFR-binding proteins may comprise any of the modified light chain domains described herein (e.g., SEQ ID NOs. 37-40, 42-46, 51-54, 56-60, 97-99) or a light chain domain having an amino acid sequence of SEQ ID NO:34; and/or any of the modified heavy chain domains described herein (e.g., SEQ ID NOs. 41, 55, 95, 96, 100-102) or a heavy chain domain having an amino acid sequence of SEQ ID NO:35; and/or a human IgG1 Fc domain, any of the modified Fc domains described herein (e.g., SEQ ID NOs. 47-50 and 61-64), or an Fc domain having an amino acid sequence of SEQ ID NO:36.

In certain embodiments, the neutral nonpolar amino acid at L₃, V₉, I₁₀ and/or L₁₅₄ of SEQ ID NO:34, or at L₁₇₆ of SEQ ID NO:35, or at L₁₉K, I₃₇K, V₆₆K and L₉₃K of SEQ ID NO:36 may be substituted for a charged or neutral polar amino acid. In some embodiments, amino acid substitution at L₁₅₄ of SEQ ID NO:34 is not lysine (K). In some embodiments, the amino acid substitution at L₁₇₆ of SEQ ID NO:35 is not K.

Antibodies

In some aspects of the invention, the CD20-binding proteins and EGFR-binding proteins of the invention may be antibodies. In some embodiments, the proteins are monoclonal antibodies such as, for example, chimeric, human or humanized monoclonal antibodies. In some embodiments, the anti-CD20 antibodies and/or the anti-EGFR antibodies of the invention may be chimeric monoclonal antibodies.

As used herein, the term “antibody” refers to a whole antibody. In some embodiments, the proteins provided herein may be antigen-binding fragments of an antibody, or single antibody chains. An antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three subdomains, C_{H1}, C_{H2} and C_{H3}. Each light chain is comprised of a light chain variable region (abbreviated herein as V_{L}) and a light chain constant region. The light chain constant region is comprised of one subdomain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

Antibodies and proteins provided herein may be described in terms of proteolytic fragments including without limitation Fv, Fab, Fab′ and F(ab′)₂ fragments. Such fragments may be prepared by standard methods (see, e.g., Coligan et al. Current Protocols in Immunology, John Wiley & Sons, 1991-1997, incorporated herein by reference). An antibody may comprise at least three proteolytic fragments (i.e., fragments produced by cleavage with papain): two Fab fragments, each containing a light chain domain and a heavy chain domain (designated herein as a “Fab heavy chain domain”) and one Fc fragment containing two Fc domains. Each light chain domain contains a V_{L} and a C_{L} subdomain, each Fab heavy chain domain contains a V_{H} and a C_{H1} subdomain, and each Fc domain contains a C_{H2} and C_{H3} subdomain.

As used herein, the term “monoclonal antibody” may refer to an antibody obtained from a single clonal population of immunoglobulins that bind to the same epitope of an antigen. Monoclonal antibodies have the same Ig gene rearrangement and thus demonstrate identical binding specificity. Methods for preparing monoclonal antibodies, as described herein, are known in the art. Monoclonal antibodies can be prepared by a variety of methods. For example, monoclonal antibodies may be made by a hybridoma method (see, e.g., Kohler et al., Nature, 1975, 256: 495, incorporated herein by reference), or may be made by recombinant DNA methods (see, e.g., U.S. Pat. No. 4,816,567, incorporated herein by reference). The monoclonal antibodies may also be isolated from phage antibody libraries. (See e.g., Clarkson et al., Nature, 1991, 352: 624-628 and Marks et al., J. Mol. Biol., 1991, 222: 581-597, incorporated herein by reference).

Human monoclonal antibodies may be made by any of the methods known in the art, including those disclosed in U.S. Pat. No. 5,567,610, U.S. Pat. No. 5,565,354, U.S. Pat. No. 5,571,893, Kozber, J. Immunol., 1984, 133: 3001, Brodeur, et al., Monoclonal Antibody Production Techniques and Applications, p. 51-63 (Marcel Dekker, Inc., new York, 1987), and Boerner et al., J. Immunol., 1991, 147: 86-95. Human antibodies may be obtained by recovering antibody-producing lymphocytes from the blood or other tissues of humans producing antibody to an antigen of interest (e.g., CD20 or EGFR). These lymphocytes can be treated to produce cells that grow independently in the laboratory under appropriate culture conditions. The cell cultures can be screened for production of antibodies to the antigen of interest and then cloned. Clonal cultures can be used to produce human monoclonal antibodies to CD20 and/or EGFR, or the genetic elements encoding the variable portions of the heavy and light chain of the antibodies can be cloned and inserted into nucleic acid vectors for production of antibodies of different types. In addition to the conventional methods for preparing human monoclonal antibodies, such antibodies may also be prepared by immunizing transgenic animals that are capable of producing human antibodies (e.g., Jakobovits, et al., PNAS USA, 1993, 90: 2551, Jakobovits, et al., Nature, 1993, 362: 255-258, Bruggermann, et al., Year in Immunol., 1993, 7:33 and U.S. Pat. No. 5,569,825).

As used herein, “humanized monoclonal antibody” may refer to monoclonal antibodies having at least human constant regions and an antigen-binding region, such as one, two or three CDRs, from a non-human species. Humanized antibodies specifically recognize antigens of interest, but will not evoke an immune response in humans against the antibody itself. As an example, murine CDRs may be grafted into the framework region of a human antibody to prepare the humanized antibody (e.g., L. Riechmann, et al., Nature, 1988, 332, 323, and M. S. Neuberger et al., Nature, 1985, 314, 268). Alternatively, humanized monoclonal antibodies may be constructed by replacing the non-CDR regions of non-human antibodies with similar regions of human antibodies while retaining the epitopic specificity of the original antibodies. For example, non-human CDRs and optionally some of the framework regions may be covalently joined to human FR and/or Fc/pFc′ regions to produce functional antibodies.

As used herein, the term “chimeric antibody” may refer to a monoclonal antibody comprising a variable region from one source (e.g., species) and at least a portion of a constant region derived from a different source. In some embodiments, chimeric antibodies are prepared by recombinant DNA techniques. In some embodiments, the chimeric antibodies comprise a murine variable region and a human constant region. Such chimeric antibodies may, in some embodiments, be the product of expressed immunoglobulin genes comprising DNA segments encoding murine immunoglobulin variable regions and DNA segments encoding human immunoglobulin constant regions. Methods for producing chimeric antibodies involve conventional recombinant DNA and gene transfection techniques (see, e.g., Morrison, S. L., et al., Proc. Natl. Acad. Sci. USA, 1984, 81: 6851-6855; U.S. Pat. No. 5,202,238; and U.S. Pat. No. 5,204,244).

Antigen-binding antibody fragments are also encompassed by the invention. Only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions of the antibody, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. An isolated F(ab′)2 fragment is referred to as a bivalent monoclonal fragment because of its two antigen binding sites. Similarly, an antibody from which the Fc region has been enzymatically cleaved, or which has been produced without the Fc region, designated an Fab fragment, retains one of the antigen binding sites of an intact antibody molecule. Further, Fab fragments consist of a covalently bound antibody light chain and a portion of the antibody heavy chain denoted Fd (heavy chain variable region, referred to herein as Fab heavy chain domain). The Fd fragments are the major determinant of antibody specificity (a single Fd fragment may be associated with up to ten different light chains without altering antibody specificity) and Fd fragments retain epitope-binding ability in isolation.

The terms Fab, Fc, pFc′, F(ab′)2 and Fv are employed with either standard immunological meanings (Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford)). Well-known functionally active antibody fragments include but are not limited to F(ab′)2, Fab, Fv and Fd fragments of antibodies. These fragments, which lack the Fc fragment of intact antibody, clear more rapidly from the circulation, and may have less non-specific tissue binding than an intact antibody (Wahl et al., J. Nucl. Med. 24:316-325 (1983)). For example, single-chain antibodies may be constructed in accordance with the methods described in U.S. Pat. No. 4,946,778. Such single-chain antibodies include the variable regions of the light and heavy chains joined by a flexible linker moiety. Methods for obtaining a single domain antibody (“Fd”) which comprises an isolated variable heavy chain single domain, also have been reported (see, e.g., Ward, et al., Nature, 1989, 341:644-646, disclosing a method of screening to identify an antibody heavy chain variable region (V_{H} single domain antibody) with sufficient affinity for its target epitope to bind thereto in isolated form). Methods for making recombinant Fv fragments based on known antibody heavy chain and light chain variable region sequences are known in the art and have been described, (see, e.g., Moore et al., U.S. Pat. No. 4,462,334). Other references describing the use and generation of antibody fragments include, e.g., Fab fragments (Tijssen, Practice and Theory of Enzyme Immunoassays (Elsevieer, Amsterdam, 1985)), Fv fragments (Hochman, et al., Biochemistry, 1973, 12: 1130; Sharon, et al., Biochemistry, 1976, 15: 1591; Ehrilch, et al., U.S. Pat. No. 4,355,023) and portions of antibody molecules (Audilore-Hargreaves, U.S. Pat. No. 4,470,925). Thus, antibody fragments may be constructed from intact antibodies without destroying the specificity of the antibodies for the CD20 or EGFR epitope.

In some embodiments, the CD20-binding proteins and the EGFR-binding proteins of the invention may be recombinant forms of antibodies. In some embodiments, the proteins may be stabilized Fv fragments having single chain Fv forms (e.g., scFv) and comprising a peptide linker joining the variable heavy chain and variable light chain domains. In other embodiments, the proteins may be Fv fragments stabilized by inter-chain disulfide linkage (e.g., dsFv). In some embodiments, the proteins may contain additional cysteine residues engineered to facilitate the conjoining of the variable heavy chain and variable light chain domains. In further embodiments, the proteins may be minibodies or single variable domain antibodies (“dAbs”). Minibodies may be genetically engineered antigen-binding constructs having structures reminiscent of antibodies (e.g., having Fab and/or Fc regions with a reduced number of variable and/or constant domains). In other embodiments, the proteins may include dimerization domains (e.g. “leucine zippers”) or other chemical modifications.

In some embodiments, the antibodies (e.g., anti-CD20 antibodies and/or anti-EGFR antibodies) may exhibit an affinity for their target that is similar to (or greater than) the affinity exhibited by existing antibodies that bind to the same target. As an example, anti-CD20 antibodies of the invention may recognize and bind to CD20 antigen on cells with an affinity at least comparable to that of rituximab. As another example, anti-EGFR antibodies of the invention may recognize and bind to EGFR on cells with an affinity at least comparable to that of cetuximab.

Conjugates

In some aspects, CD20-binding proteins and the EGFR-binding proteins of the invention may be a conjugate. As used herein, the term “conjugate” may refer two or more compounds which are linked together so that at least some of the properties from each compound are retained in the conjugate. Linking may be achieved by a covalent or non-covalent bond. In some embodiments, the compounds of the conjugate are linked via a covalent bond. The different compounds of a conjugate may be directly bound to each other via one or more covalent bonds between atoms of the compounds. Alternatively, the compounds may be bound to each other via a linker molecule wherein the linker is covalently attached to atoms of the compounds. If the conjugate is composed of more than two compounds, then these compounds may be, for example, linked in a chain conformation, one compound attached to the next compound, or several compounds each may be attached to one central compound.

Conjugates may comprise additional agents that are useful in therapy, diagnosis, prognosis and/or monitoring of a condition such as, for example, cancer. Examples of additional agents include, without limitation, antibodies or fragments of antibodies, enzymes, interaction domains, stabilizing domains, signaling sequences, detectable labels, fluorescent dyes, toxins, catalytic antibodies, cytolytic components, immunomodulators, immunoeffectors, MHC class I or class II antigens, chelators for radioactive labeling, radioisotopes, liposomes, transmembrane domains, viruses, and cells. In some embodiments, the additional agents are radionuclides or a cytotoxic agents capable of killing cells (e.g., cancer cells), such as chemotherapeutic agents. Examples of other agents that may be used include alkylating agents such as cisplatin, anti-metabolites, plant alkaloids and terpenoids, vinca alkaloids, podophyllotoxin, taxanes such as taxol, topoisomerase inhibitors such as irinotecan and topotecan, and/or antineoplastics such as doxorubicin.

### Nucleic Acids

In some aspects, provided herein are nucleic acids that encode the CD20-binding proteins and the EGFR-binding proteins of the invention. As used herein, the term “nucleic acid” may refer to single-stranded and double-stranded nucleic acids and ribonucleic acids as well as deoxyribonucleic acids. In some embodiments, the nucleic acids are double-stranded DNA such as cDNA or single-stranded RNA such as mRNA. Nucleic acids may comprise naturally occurring and/or synthetic nucleotides and can be naturally or synthetically modified, for example by methylation, 5′- and/or 3′-capping. The sequence of the nucleic acids may have any nucleotide sequence suitable for encoding the proteins of the invention. The nucleic acids may be one consecutive nucleic acid molecule or they may be composed of several nucleic acid molecules, each coding for a different part of the protein of the invention. In some embodiments, the nucleic acid sequences may be at least partially adapted to a specific codon usage, for example, human codon usage, of the host cells or organisms in which the nucleic acids are to be expressed. The nucleic acids may be double-stranded or single-stranded DNA or RNA.

If the antibodies of the invention are single chain constructs, the nucleic acids encoding them may be single nucleic acid molecules containing a coding region which codes for the entire antibody. If the antibodies are composed of more than one amino acid chain, the nucleic acids may be, for example, single nucleic acid molecules containing several coding regions each coding for one of the amino acid chains of the antibodies. In some embodiments, the coding regions may be separated by regulatory elements such as IRES elements in order to generate separate amino acid chains. In some embodiments, the nucleic acids may be composed of several nucleic acid molecules wherein each nucleic acid molecule may comprise one or more coding regions, each coding for one of the amino acid chains of the antibodies. The nucleic acids may also comprise additional nucleic acid sequences or other modifications which, for example, may code for other proteins, may influence the transcription and/or translation of the coding region(s), may influence the stability or other physical or chemical properties of the nucleic acid, or may have no function at all.

### Vectors and Expression Cassettes

In some aspects, provided herein are vectors and/or expression cassettes comprising the proteins of the invention.

As used herein, the term “vector” may refer to any intermediary vehicle for a nucleic acid which enables the nucleic acid, for example, to be introduced into prokaryotic and/or eukaryotic cells and, in some instances, to be integrated into a genome. Vectors of this kind may be replicated and/or expressed in the cells. Vectors may comprise plasmids, phagemids, bacteriophages or viral genomes. The term “plasmid,” as used herein, may refer to a construct of extrachromosomal genetic material, for example, a circular DNA duplex, which can replicate independently of chromosomal DNA.

As used herein, the term “expression cassette” may refer to nucleic acid constructs which are capable of enabling and regulating the expression of coding nucleic acid sequences introduced therein. Expression cassettes may comprise promoters, ribosome binding sites, enhancers and/or other control elements which regulate transcription of a gene or translation of an mRNA. The exact structure of expression cassettes may vary as a function of the species or cell type, but generally comprises 5′-untranscribed and 5′- and 3′-untranslated sequences which are involved in initiation of transcription and translation, respectively, such as TATA box, capping sequence, CAAT sequence, and the like. More specifically, 5′-untranscribed expression control sequences may comprise a promoter region, which includes a promoter sequence for transcriptional control of operatively connected nucleic acids. Expression cassettes may also comprise enhancer sequences or upstream activator sequences.

As used herein, the term “promoter” refers to nucleic acid sequences which are located upstream (5′) of the nucleic acid sequences which are to be expressed and control expression of the sequence by providing a recognition and binding site for RNA-polymerases. Promoters may include further recognition and binding sites for additional factors which may be involved in the regulation of gene transcription. Promoters may control the transcription of a prokaryotic or eukaryotic gene. Furthermore, promoters may be inducible, i.e., initiate transcription in response to an inducing agent, or may be constitutive if transcription is not controlled by an inducing agent. Genes which are under the control of inducible promoters are not expressed or are only expressed to a small extent if inducing agents are absent. In the presence of inducing agents the genes are switched on or the level of transcription is increased. This is mediated, in general, by binding of a specific transcription factor.

In addition, the expression cassettes or vectors may comprise other elements, for example, elements which may influence and/or regulate transcription and/or translation of the nucleic acids, amplification and/or reproduction of the expression cassettes or vectors, integration of the expression cassettes or vectors into the genome of host cells, and/or copy number of the expression cassettes or vectors in host cells. Suitable expression cassettes and vectors comprising respective expression cassettes for expressing proteins (e.g., antibodies) are well known.

### Host Cells

In some embodiments, the CD20-binding proteins and EGFR-binding proteins of the invention are produced by the host cells or cell lines as described above.

In some aspects, the invention provides host cells comprising the nucleic acids provided herein or the expression cassettes or vectors provided herein. The nucleic acids may be present in the form of a single copy or of two or more copies and, in some embodiments, are expressed in the host cells. As used herein, the term “host cell” may refer to any cell which can be transformed or transfected with an exogenous nucleic acid. They may be isolated cells or cells comprised in a tissue. The cells may be derived from a multiplicity of tissue types and may comprise primary cells and cell lines. Host cells may be prokaryotic (e.g., E. coli) or eukaryotic (e.g., mammalian, in particular human, yeast and/or insect).

In some embodiments, host cells are bacterial cells such as E. coli, yeast cells such as a Saccharomyces cells (e.g., S. cerevisiae), insect cells such as a Sf9 cells, or mammalian cells such as human cells, for example, tumor-derived human cells, hamster cells (e.g., Chinese Hamster Ovary cells), or primate cells. In some embodiments, the host cells are derived from human myeloid leukemia cells. Examples of cell lines for use herein include, without limitation, K562, KG1, MUTZ-3, NM-F9, NM-D4, NM-H9D8, NM-H9D8-E6, NM H9D8-E6Q12, GT-2X, and cells or cell lines derived from any of these host cells, or mixture of cells or cell lines comprising at least one of those cells. These cell lines and their properties are described in detail in the PCT application WO 2008/028686 A2.

In some embodiments, the host cells are optimized for expression of glycoproteins, in particular antibodies, having a specific glycosylation pattern. In some embodiments, the codon usage in the coding region of the nucleic acids and/or the promoters and the additional elements of the expression cassettes or vectors are compatible with and, in some instances, optimized for the type of host cell used.

### Compositions

The invention also provides compositions comprising any of the CD20-binding proteins or EGFR-binding proteins, the nucleic acids, the expression cassettes and/or vectors, the host cells, or the conjugates described herein. The compositions may also contain more than one of these components. Furthermore, the compositions may comprise one or more additional components selected from buffers, solubilizers, surfactants, carriers, excipients, solvents, and/or diluents.

In some aspects of the invention, the compositions may comprise CD20-binding proteins comprising: a light chain domain having an amino acid sequence of SEQ ID NO:1, or a light chain domain having an amino acid sequence of SEQ ID NO:1 modified to include at least one amino acid substitution selected from a neutral polar amino acid at V₃, a neutral polar amino acid at A₉, a neutral polar amino acid at I₁₀, a positive polar amino acid at I₁₀, a neutral polar amino acid at V₅₉, a negative polar amino acid at V₅₉, a positive polar amino acid at V₁₀₉, and a negative polar amino acid at L₁₅₃; and/or a heavy chain domain having an amino acid sequence of SEQ ID NO:2, or a heavy chain domain having an amino acid sequence of SEQ ID NO:2 modified to include at least one amino acid substitution selected from a neutral polar amino acid at Y₁₀₁, a neutral polar amino acid at L₁₇₈, and a positive polar amino acid at L₁₇₈; and/or a human IgG Fc domain, an Fc domain having an amino acid sequence of SEQ ID NO:3, or an Fc domain having an amino acid sequence of SEQ ID NO:3 modified to include at least one amino acid substitution selected from a positive polar amino acid at position L₁₅, a positive polar amino acid at position I₃₃, a positive polar amino acid at position V₆₂, and a positive polar amino acid at position L₈₉.

In some embodiments, the compositions may comprise CD20-binding proteins comprising: a light chain domain having an amino acid sequence of SEQ ID NO:1 modified to include at least one amino acid substitution selected from V₃Q, A₉S, I₁₀S, I₁₀K, V₅₉S, V₅₉D, V₁₀₉K and L₁₅₃D; and/or a heavy chain domain having an amino acid sequence of SEQ ID NO:2 modified to include at least one amino acid substitution selected from a neutral polar amino acid at Y₁₀₁, a neutral polar amino acid at L₁₇₈, and a positive polar amino acid at L₁₇₈; and/or a human IgG Fc domain or an Fc domain having an amino acid sequence of SEQ ID NO:3 modified to include at least one amino acid substitution selected from L₁₅K, I₃₃K, V₆₂K and L₈₉K.

In some aspects of the invention, the compositions may comprise EGFR-binding proteins comprising a light chain domain having an amino acid sequence of SEQ ID NO:34, or a light chain domain having an amino acid sequence of SEQ ID NO:34 modified to include at least one amino acid substitution selected from a neutral polar amino acid at L₃, a positive polar amino acid at L₃, a neutral polar amino acid at V₉, a negative polar amino acid at V₉, a neutral polar amino acid at I₁₀, a positive polar amino acid at L₁₅₄, and a negative polar amino acid at L₁₅₄; and/or a heavy chain domain having an amino acid sequence of SEQ ID NO:35, or a heavy chain domain having an amino acid sequence of SEQ ID NO:35 modified to include at least one amino acid substitution selected from a positive polar amino acid at L₉₉, a positive polar amino acid at L₁₁₄, a positive polar amino acid at L₁₇₆, and a neutral polar amino acid at L₁₇₆; and/or a human IgG Fc domain, an Fc domain having an amino acid sequence of SEQ ID NO:36, or an Fc domain having an amino acid sequence of SEQ ID NO:36 modified to include at least one amino acid substitution selected from a positive polar amino acid at position L₁₉, a positive polar amino acid at position I₃₇, a positive polar amino acid at position V₆₆, and a positive polar amino acid at position L₉₃.

In some embodiments, the compositions may comprise EGFR-binding proteins comprising: a light chain domain having an amino acid sequence of SEQ ID NO:34 modified to include at least one amino acid substitution selected from L₃Q, L₃K, V₉S, V₉D, I₁₀S, L₁₅₄K and L₁₅₄D; and/or a heavy chain domain having an amino acid sequence of SEQ ID NO:35 modified to include at least one amino acid substitution selected from L₉₉K, L₁₁₄K, L₁₇₆K and L₁₇₆5; and/or a human IgG Fc domain or an Fc domain having an amino acid sequence of SEQ ID NO:36 modified to include at least one amino acid substitution selected from L₁₉K, I₃₇K, V₆₆K and L₉₃K.

In some embodiments, the compositions may comprise the proteins provided herein at a concentration of about 20 mg/ml to about 350 mg/ml. In some embodiments, the protein concentration may be about 30 mg/ml to about 340 mg/ml, about 40 mg/ml to about 330 mg/ml, about 50 mg/ml to about 320 mg/ml, about 60 mg/ml to about 310 mg/ml, about 70 mg/ml to about 300 mg/ml, about 80 mg/ml to about 290 mg/ml, about 90 mg/ml to about 280 mg/ml, about 100 mg/ml to about 270 mg/ml, 110 mg/ml to about 260 mg/ml, 120 mg/ml to about 250 mg/ml, 130 mg/ml to about 240 mg/ml, 140 mg/ml to about 230 mg/ml, 150 mg/ml to about 220 mg/ml, 160 mg/ml to about 210 mg/ml, 170 mg/ml to about 200 mg/ml, or about 180 mg/ml to about 190 mg/ml. In some embodiments, the protein concentration is greater than 100 mg/ml. In some embodiments, the protein concentration is 120±20 mg/ml. In some embodiments, the protein concentration is about 50 mg/ml, about 75 mg/ml, about 100 mg/ml, about 110 mg/ml, about 120 mg/ml, about 130 mg/ml, about 140 mg/ml, or about 150 mg/ml. In some embodiments, the protein concentration is 110 mg/ml, 120 mg/ml, 130 mg/ml, 140 mg/ml, or 150 mg/ml. In some embodiments, the concentration of the CD20-binding proteins in the compositions is greater than the concentration of rituximab (RITUXAN®), formulated for intravenous routes of administration. In other embodiments, the concentration of the EGFR-binding proteins in the compositions is greater than the concentration of cetuximab (ERBITUX®), formulated for intravenous routes of administration.

In some embodiments, the compositions provided herein may comprise additional components such as, for example, at least one buffer, at least one stabilizer and/or at least one surfactant.

Examples of buffers that may be used in the compositions provided herein include, without limitation, histidine buffer, acetic acid buffer, citric acid buffer, L-histidine/HCl buffer, and combinations thereof. The buffer(s) may be present at a concentration of about 1 mM to about 100 mM. In some embodiments, the buffer(s) may be present at a concentration of about 1 mM, about 10 mM, about 20 mM, about 30 mM, about 40 mM, about 50 mM, about 60 mM, about 70 mM, about 80 mM, about 90 mM or about 100 mM.

The pH of the compositions of the invention may be adjusted to about 4.5 to about 7.0. In some embodiments, the pH is 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7. In some embodiments, the pH is about 5.5. In some embodiments, the pH is 5.5±2. In some embodiments, the pH of the composition is adjusted independently from the buffer used. This pH may be obtained by adjustment with an acid or a base, or by using adequate buffer (or mixtures of buffer), or both.

Examples of stabilizers that may be used, e.g., as a primary stabilizer, in the compositions provided herein include, without limitation, salt (e.g., NaCl), carbohydrate, saccharide, amino acid(s) (e.g., methionine), and sugar (e.g., α,α-trehalose dehydrate). Other examples of stabilizers that may be used, e.g, as a secondary stabilizer, in the compositions provided herein include, without limitation, arginine, ornithine, lysine, histidine, glutamic acid, aspartic acid, isoleucine, leucine, alanine, phenylalanine, tyrosine, tryptophan, methionine, serine or proline (e.g., as a hydrochloride). The stabilizer(s) may be present at a concentration of about 5 mM to about 500 mM, about 5 mM to about 25 mM, about 15 mM to about 250 mM, about 100 mM to about 500 mM, or about 150 mM to about 250 mM. In some embodiments, the stabilizer(s) may be present at a concentration of about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, or about 500 mM. In some embodiments, the composition may comprise methionine at a concentration of about 5 mM to about 25 mM, or about 5 mM to about 15 mM. In some embodiments, the methionine concentration is about 10 mM.

Examples of surfactants that may be used in the compositions provided herein include, without limitation, nonionic surfactant(s) such as, for example, polysorbate (e.g., polysorbate 20 and polysorbate 80) and polyethylene-polypropylene copolymer. The surfactant(s) may be present at a concentration of about 0.01% to about 0.1% (weight/volume, w/v), about 0.02% to about 0.08% (w/v), about 0.02% to 0.06% (w/v), or about 0.06% (w/v). In some embodiments, the surfactant(s) may be present at a concentration of about 0.02% (w/v), about 0.03% (w/v), about 0.04% (w/v), about 0.05% (w/v), about 0.06% (w/v), about 0.07% (w/v), about 0.08% (w/v), about 0.09% (w/v), or about 0.1% (w/v).

In some embodiments, the CD20-binding proteins or the EGFR-binding proteins of the compositions provided herein may be lyophilized or in aqueous solution.

In some embodiments, the compositions may or may not be sterile. A sterile composition may be one that is aseptic or free from all living microorganisms and their spores. In some embodiments, the compositions provided herein may further comprise pharmaceutically acceptable carriers, excipients and/or stabilizers (see, e.g., Remington's Pharmaceutical Sciences 16th edition, Osol, A. Ed., 1980, incorporated herein by reference). As used herein, pharmaceutically acceptable carriers may include without limitation solvents, dispersion media, coatings, antibacterial and antifungal agents, and isotonic and absorption delaying agents. Pharmaceutically acceptable carriers, excipients and/or stabilizers are non-toxic to recipients (e.g., human subjects) at the dosages and concentrations used. In some embodiments, the carriers, excipients and/or stabilizers are suitable for intravenous, intramuscular, subcutaneous, parenteral, spinal or epidermal administration (e.g., by injection or infusion).

Examples of carriers, excipients and/or stabilizers include, without limitation, buffers such as phosphate, citrate, and/or other organic acids; antioxidants such as ascorbic acid and/or methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl parabens, catechol, resorcinol, cyclohexanol, 3-pentanol, and/or m-cresol; low molecular weight (e.g., less than about 10 residues) polypeptides; proteins such as serum albumin, gelatin, and/or immunoglobulins; hydrophilic polymers such as olyvinylpyrrolidone; amino acids such as glycine, glutamine, asparagine, histidine, arginine, and/or lysine; monosaccharides, disaccharides, and/or other carbohydrates including glucose, mannose, and/or dextrins; chelating agents such as EDTA; sugars such as sucrose, mannitol, trehalose and/or sorbitol; salt-forming counter-ions such as sodium; metal complexes such as Zn-protein complexes; and/or non-ionic surfactants such as TWEEN®, PLURONIC® and/or polyethylene glycol (PEG).

In some embodiments, the compositions may further comprise adjuvants such as, for example, preservatives, wetting agents, emulsifying agents and/or dispersing agents. In some embodiments, the compositions are sterilized and/or may comprise antibacterial and antifungal agents, for example, paraben, chlorobutanol, and/or phenol sorbic acid. In some embodiments, the composition may also comprise isotonic agents, such as sugars and/or sodium chloride. In some embodiments, the composition may further comprise agents that delay absorption such as aluminum monostearate and/or gelatin.

In some embodiments, the compositions provided herein may further comprise at least one additional active agent including, without limitation, cytotoxic agents, chemotherapeutic agents, cytokines and/or immunosuppressive agents.

In some embodiments, a composition provided herein may be substantially free of any additive that reduces aggregation of the CD20-binding proteins or the EGFR-binding proteins provided herein.

In some embodiments, the compositions provided herein may comprise CD20-binding proteins or EGFR-binding proteins entrapped in microcapsules prepared, for example, by coacervation techniques (e.g., hydroxymethylcellulose or gelatin-microcapsules) or by interfacial polymerization (e.g., poly-(methylmethacylate) microcapsules). In some embodiments, the compositions provided herein may comprise CD20-binding proteins or EGFR-binding proteins entrapped in colloidal drug delivery systems such as, for example, liposomes, albumin microspheres, microemulsions, nano-particles and nanocapsules. In some embodiments, the compositions provided herein may comprise CD20-binding proteins or EGFR-binding proteins entrapped in macroemulsions. See, e.g., Remington's Pharmaceutical Sciences 16th edition, Osol, A. Ed. (1980).

In some embodiments, the compositions provided herein may be sustained-release compositions.

In various aspects of the invention, provided herein are highly concentrated, stable liquid compositions of CD20-binding proteins or EGFR-binding proteins having a reduced tendency to aggregate. As used herein, “highly concentrated” compositions may refer to compositions comprising the proteins provided herein at a concentration of greater than 100 mg/ml (e.g., about 110 mg/ml to about 150 mg/ml). As used herein, “stable” compositions may refer to compositions comprising the proteins provided herein that retain (or essentially retain) physical stability and/or chemical stability and/or biological activity upon storage for an intended period of time (e.g., intended shelf-life of the composition) at an intended temperature (e.g., 2-8° C.). In some embodiments, the compositions may be stable following freezing (to, e.g., −20° C.) and thawing of the compositions, for example, following one or more cycles of freezing and thawing. Various analytical techniques for measuring protein stability are available in the art (see, e.g., Peptide and Protein Drug Delivery, 247-301, Vincent Lee Ed., Marcel Dekker, Inc., New York, N.Y., Pubs., 1991; and Jones, A. Adv. Drug Delivery Rev., 1993, 10: 29-90, each of which are incorporated herein by reference). Stability may be measured at a select temperature for a select time period. Stability may be evaluated qualitatively and/or quantitatively in a variety of different ways including, without limitation, evaluation of aggregate formation (e.g., using size exclusion chromatography, measuring turbidity, and/or visual inspection); assessment of charge heterogeneity using cation exchange chromatography or capillary zone electrophoresis; SDS-PAGE analysis to compare reduced and intact antibody; and/or evaluating biological activity or antigen binding function of the protein. Instability may involve aggregation, deamidation (e.g. Asn deamidation), oxidation (e.g., Met oxidation), isomerization (e.g. Asp isomeriation), clipping/hydrolysis/fragmentation (e.g,. hinge region fragmentation), succinimide formation, and/or unpaired cysteine(s).

In some embodiments, the compositions, for example, the highly concentrated compositions, are formulated for subcutaneous or intramuscular delivery. In some embodiments, the compositions provided herein are considered to be highly concentrated compositions formulated for subcutaneous delivery if the protein concentration in the compositions is greater than 100 mg/ml (e.g., about 110 mg/ml to about 150 mg/ml) and the volume of the compositions is about or less than 2 ml. In some embodiments, the volume of the compositions is about 1.5 ml.

Examples of highly concentrated compositions comprising the CD20-binding proteins or the EGFR-binding proteins provided herein include, without limitation, the following:

(a) 100 to 150 mg/ml protein, 1 to 50 mM of a histidine buffer (e.g., L-histidine/HCl) at a pH of about 5.5, 15 to 250 mM of a stabilizer (e.g., α,α-trehalose dihydrate) and, optionally, methionine as a second stabilizer at a concentration of 5 to 25 mM, and polysorbate 20 or polysorbate 80 at a concentration of 0.02 to 0.06% (w/v);

(b) 120±20 mg/ml protein, 10 to 30 mM or 20 mM of a histidine buffer (e.g., L-histidine/HCl) at a pH of about 5.5, 150 to 250 mM or 210 mM of a stabilizer (e.g., α,α-trehalose dihydrate) and, optionally, methionine as a second stabilizer at a concentration of 5 to 25 mM, 5 to 15 mM or 10 mM, and polysorbate 20 or polysorbate 80 at a concentration of 0.02 to 0.06% (w/v);

(c) 120 mg/ml protein, 10 to 30 mM or 20 mM of a histidine buffer (e.g., L-histidine/HCl) at a pH of about 5.5, 150 to 250 mM or 210 mM of a stabilizer (e.g., α,α-trehalose dihydrate) and, optionally, methionine as a second stabilizer at a concentration of 5 to 25 mM, 5 to 15 mM, or 10 mM, and polysorbate 20 or polysorbate 80 at a concentration of 0.02 to 0.06% (w/v); and

(d) 120 mg/ml protein, 20 mM of L-histidine/HCl at a pH of about 5.5, 210 mM α,α-trehalose dihydrate and, optionally, 10 mM methionine as a second stabilizer, and polysorbate 20 or polysorbate 80 at a concentration of 0.02 to 0.06% (w/v).

### Methods of Use

The invention also provides methods of using the CD20-binding and EGFR-binding proteins and composition, for example, in in vitro, in situ and in vivo applications.

Examples of in vitro and in situ applications in accordance with the invention include, without limitation, cell killing assays, as positive controls for apoptosis assays, for purification or immunoprecipitation of antigen from cells, and for diagnostic assays.

Examples of in vivo applications in accordance with the invention include, without limitation, methods of treatment. In some embodiments, the proteins and protein compositions of the invention may be used to treat one or more condition(s) such as, for example, cancers and/or non-malignant conditions in a subject in need thereof. Thus, in some embodiments, provided herein are methods of treating cancers and/or non-malignant conditions in a subject in need thereof. In some embodiments, the methods of treatment comprise administering to the subject in need thereof a therapeutically effective amount of the proteins and/or compositions of the invention. The term “therapeutically effective amount” is described below.

The proteins and protein compositions of the invention may also be used for the preparation of a medicament for treating cancers and non-malignant conditions in a subject.

As used herein, a subject is a warm-blooded mammal and includes, for instance, humans, primates, horses, cows, swine, goats, sheep, dogs, and cats. In some embodiments, the subject is a non-rodent subject. A non-rodent subject is any subject as defined above, but specifically excluding rodents such as mice, rats, and rabbits. In some embodiments, the subject is a human.

In some embodiments, the proteins provided herein are administered at dosages of about 0.0001 mg/kg to about 100 mg/kg of the recipient (e.g., subject) body weight. In some embodiments, the proteins are administered at dosages of about 0.01 mg/kg to about 5 mg/kg of the recipient body weight. In some embodiments, the proteins are administered at dosages of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg.kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, or 10 mg/ml recipient body weight. In some embodiments, the proteins are administered at dosages of about 0.3 mg/kg body weight, about 1 mg/kg recipient body weight, about 3 mg/kg recipient body weight, about 5 mg/kg recipient body weight, about 10 mg/kg recipient body weight, or within the range of 1-10 mg/kg recipient body weight.

In some embodiments, the proteins provided herein are administered once per week, once every two weeks, once every three weeks, once every four weeks, once a month, once every three months or once every three to six months.

Dosage and frequency may vary depending on the half-life of the protein in the recipient. For example, in general, human antibodies show the longest half-life, followed by humanized antibodies, chimeric antibodies, and nonhuman antibodies.

Actual dosage levels of the proteins provided herein may be varied so as to obtain an amount of the proteins which is effective to achieve the desired response (e.g., therapeutic response) for a particular subject, composition, and mode of administration, without being toxic to the subject. The selected dosage level may depend upon a variety of pharmacokinetic factors including, without limitation, the activity of the proteins, the route of administration, the time of administration, the rate of excretion of the proteins, the duration of administration, other drugs, compounds and/or materials used in combination with the proteins, the age, sex, weight, condition, general health and prior medical history of the subject.

In some embodiments, the proteins and protein compositions provided herein may be administered at therapeutically effective amounts. As used herein, “therapeutically effective amount” of the proteins and/or the protein compositions may result in a decrease in severity of the symptoms of a condition, an increase in frequency and duration of symptom-free periods, or a prevention of impairment or disability due to the condition. For example, for administration of the proteins or compositions of the invention to a subject having tumors, a “therapeutically effective amount” inhibits cell growth or tumor growth by at least about 20%, by at least about 40%, by at least about 60%, or by at least about 80% relative to untreated subjects. The ability of the proteins to inhibit tumor growth may be evaluated in an animal model system predictive of efficacy in human tumors. Alternatively, the proteins may be evaluated by examining the ability of the proteins to inhibit tumor growth in vitro. In some embodiments, a therapeutically effective amount of the CD20-binding proteins or the EGFR-binding proteins may decrease tumor size, or otherwise ameliorate symptoms in a subject. One of ordinary skill in the art would be able to determine such amounts based on such factors as the subject's size, the severity of the subject's symptoms, and the particular composition or route of administration selected.

In some embodiments, the proteins and/or the compositions provided herein may be administered via one or more routes of administration using one or more of a variety of methods known in the art. In some embodiments, the proteins and/or the protein compositions are administered via subcutaneous, intravenous, intramuscular, intradermal, intraperitoneal, spinal or other parenteral routes of administration, for example by injection or infusion. As used herein, the term “parenteral administration” may refer to modes of administration other than enteral and topical administration, usually by injection, and includes, without limitation, subcutaneous, intravenous, intramuscular, intraarterial, intrathecal, intracapsular, intraorbital, intracardiac, intradermal, intraperitoneal, transtracheal, subcuticular, intraarticular, subcapsular, subarachnoid, intraspinal, epidural and intrasternal injection, and infusion. In other embodiments, the proteins and/or the protein compositions may be administered via a nonparenteral route, such as a topical, epidermal or mucosal route of administration, for example, intranasally, orally, vaginally, rectally, sublingually or topically.

Uses for CD20-Binding Proteins

In some embodiments, the CD20-binding proteins and compositions may be used to treat cancers such as, for example, B cell lymphoma, B cell leukemia, non-Hodgkin's lymphoma, lymphocyte predominant Hodgkin's disease, chronic lymphocytic leukemia and small lymphocytic lymphoma and/or other cancers expressing CD20. In some embodiments, the CD20-binding proteins and compositions may be used to treat non-malignant condition such as autoimmune conditions, for example, rheumatoid arthritis, juvenile rheumatoid arthritis, systemic lupus erythematosus, lupus nephritis, ulcerative colitis, Wegener's disease, inflammatory bowel disease, idiopathic thrombocytopenic purpura, thrombotic thrombocytopenic purpura, autoimmune thrombocytopenia, multiple sclerosis, psoriasis, IgA nephropathy, IgM polyneuropathies, myasthenia gravis, vasculitis, diabetes mellitus, Reynaud's syndrome, Sjorgen's syndrome and/or glomerulonephritis.

In some embodiments, the CD20-binding proteins and compositions may be used to induce apoptosis in B cells. In some embodiments, a method of inducing apoptosis in B cells comprises contacting the B cells with the CD20-binding proteins or compositions of the invention.

Uses for EGFR-Binding Proteins

In some embodiments, the EGFR-binding proteins and compositions may be used to treat cancers such as, for example, brain tumors, tumors of the urogenital tract, tumors of the lymphatic system, stomach tumors, laryngeal tumors, monocytic leukemia, lung adenocarcinoma, small-cell lung carcinoma, pancreatic cancer, glioblastoma, breast carcinoma, and/or other cancers expressing EGFR. In some embodiments, the EGFR-binding proteins and compositions may be used to treat non-malignant tumors.

### Kits

Other aspects of the invention provide kits comprising the CD20-binding or EGFR-binding proteins and compositions described herein. In some embodiments, kits may comprise a single container (e.g., a syringe or vial) containing a protein or composition of the invention.

In some embodiment, the volume of the composition may be less than or equal to 2 ml. In such embodiments, the concentration of the protein is greater than 100 mg/ml, for example about 110 mg/ml, about 120 mg/ml, about 130 mg/ml, about 140 mg/ml, or about 150 mg/ml. The kits may comprise a container(s) and a label or package insert on or associated with the container. Suitable containers include without limitation bottles, vials, and syringes. The containers may be formed from a variety of materials such as glass or plastic. The containers may hold a composition which may be administered to a subject and may have a sterile access port (for example the container may be an intravenous solution bag or a vial having a stopper pierceable by a hypodermic injection needle).

The label or package insert may indicate that kits are used for administering the proteins or protein compositions to a subject. The label or package insert may further comprise instructions for administration. As used herein, a “package insert” may refer to instructions customarily included in commercial packages of products that contain information about the product and its use, for example, indications, dosage, administration, contraindications and/or warnings concerning the use of such products. In some embodiments, the package insert may indicate that the proteins and protein compositions are used for treating cancer or autoimmune conditions.

Additionally, the kits may further comprise other containers comprising additional components including, without limitation, pharmaceutically-acceptable buffers, bacteriostatic water for injection (BWFI), phosphate-buffered saline, Ringer's solution and/or dextrose solution. The kits may also comprise other materials including, without limitation, other buffers, diluents, filters, needles and/or syringes.

In some embodiments, provided herein are kits that may be useful for various purposes, e.g., for cell killing assays, as a positive control for apoptosis assays, for purification or immunoprecipitation of antigen from cells. For isolation and purification of antigen, the kit may contain an antibody coupled to beads (e.g., sepharose beads). Kits may contain the antibodies for detection and quantitation of CD20 or EGFR in vitro, e.g., in an ELISA or a Western blot.

The invention is further described by the following numbered paragraphs:

1. A CD20-binding protein comprising:

a light chain domain having an amino acid sequence of SEQ ID NO:1 modified to include at least one amino acid substitution selected from a neutral polar amino acid at V₃, a neutral polar amino acid at A₉, a neutral polar amino acid at I₁₀, a positive polar amino acid at I₁₀, a neutral polar amino acid at V₅₉, a negative polar amino acid at V₅₉, a positive polar amino acid at V₁₀₉, and a negative polar amino acid at L₁₅₃; and/or

a heavy chain domain having an amino acid sequence of SEQ ID NO:2 modified to include at least one amino acid substitution selected from a neutral polar amino acid at Y₁₀₁, a neutral polar amino acid at L₁₇₈, and a positive polar amino acid at L₁₇₈; and/or

an Fc domain having an amino acid sequence of SEQ ID NO:3 modified to include at least one amino acid substitution selected from a positive polar amino acid at position L₁₅, a positive polar amino acid at position I₃₃, a positive polar amino acid at position V₆₂, and a positive polar amino acid at position L₈₉.

2. The CD20-binding protein of paragraph 1, wherein the protein comprises a light chain domain having an amino acid sequence of SEQ ID NO:1 modified to include at least two, at least three, at least four, at least five, or at least six amino acid substitutions selected from a neutral polar amino acid at V₃, a neutral polar amino acid at A₉, a neutral polar amino acid at I₁₀, a positive polar amino acid at I₁₀, a neutral polar amino acid at V₅₉, a negative polar amino acid at V₅₉, a positive polar amino acid at V₁₀₉, and a negatively charged polar amino acid at L₁₅₃.
3. The CD20-binding protein of paragraph 1 or 2, wherein the neutral polar amino acid at V₃, A₉, I₁₀ and/or V₅₉ of SEQ ID NO:1 and/or at Y₁₀₁ and/or L₁₇₈ of SEQ ID NO:2 is asparagine (N), cysteine (C), glutamine (Q), histidine (H), serine (S), threonine (T) or tyrosine (Y).
4. The CD20-binding protein of any of paragraphs 1-3, wherein the negative polar amino acid at V₅₉ and/or L₁₅₃ of SEQ ID NO:1 is aspartic acid (D) or glutamic acid (E).
5. The CD20-binding protein of any of paragraphs 1-4, wherein the positive polar amino acid at I₁₀ and/or V₁₀₉ of SEQ ID NO:1, at L₁₇₈ of SEQ ID NO:2, and/or at L₁₅, I₃₃, V₆₂ and/or L₈₉ of SEQ ID NO:3 is arginine (R) or lysine (K).
6. The CD20-binding protein of any of paragraphs 1-5, wherein the protein comprises a light chain domain having an amino acid sequence of SEQ ID NO:1, a heavy chain domain having an amino acid sequence of SEQ ID NO:2, a human IgG Fc domain, or an Fc domain having an amino acid sequence of SEQ ID NO:3.
7. The CD20-binding protein of paragraph 1, wherein the protein comprises a light chain domain having an amino acid sequence of SEQ ID NO:19, SEQ ID NO:20, SEQ ID NO:21, SEQ ID NO:22, SEQ ID NO:23, SEQ ID NO:25, SEQ ID NO:26, SEQ ID NO:27, SEQ ID NO:28, SEQ ID NO:29 or SEQ ID NO:90.
8. The CD20-binding protein of paragraph 1, wherein the protein comprises a heavy chain domain having an amino acid sequence of SEQ ID NO:24 or SEQ ID NO:88.
9. The CD20-binding protein of paragraph 1, wherein the protein comprises an Fc chain domain having an amino acid sequence of SEQ ID NO:30, SEQ ID NO:31, SEQ ID NO:32 or SEQ ID NO:33.
10. A CD20-binding protein comprising:

a light chain domain having an amino acid sequence of SEQ ID NO:1 modified to include at least one amino acid substitution selected from V₃Q, A₉S, I₁₀S, I₁₀K, V₅₉S, V₅₉D, V₁₀₉K and L₁₅₃D; and/or

a heavy chain domain having an amino acid sequence of SEQ ID NO:2 modified to include at least one amino acid substitution selected from Y₁₀₁H, L₁₇₈S and L₁₇₈K; and/or

an Fc domain having an amino acid sequence of SEQ ID NO:3 modified to include at least one amino acid substitution selected from L₁₅K, I₃₃K, V₆₂K and L₈₉K.

11. The CD20-binding protein of paragraph 10, wherein the protein comprises a light chain domain having an amino acid sequence of SEQ ID NO:1 modified to include at least two, at least three, at least four, at least five, or at least six amino acid substitutions selected from V₃Q, A₉S, I₁₀S, I₁₀K, V₅₉S, V₅₉D, V₁₀₉K and L₁₅₃D.
12. The CD20-binding protein of paragraph 11, wherein the protein comprises a heavy chain domain having an amino acid sequence of SEQ ID NO:2 modified to include an amino acid substitution Y₁₀₁H and an amino acid substitution selected from L₁₇₈S and L₁₇₈K.
13. The CD20-binding protein of paragraph 12, wherein the amino acid sequence of SEQ ID NO:1 is modified to include amino acid substitutions

(a) A₉S and I₁₀S,

(b) V₃Q, A₉S and V₅₉S,

(c) V₃Q, A₉S, I₁₀S and V₅₉S,

(d) V₃Q, A₉S, V₅₉5 and L₁₅₃D, or

(e) V₃Q, A₉S, I₁₀S, V₅₉5 and L₁₅₃D.

14. The CD20-binding protein of any of paragraphs 10-13, wherein the protein comprises a light chain domain having an amino acid sequence of SEQ ID NO:1, a heavy chain domain having an amino acid sequence of SEQ ID NO:2, a human IgG Fc domain, or an Fc domain having an amino acid sequence of SEQ ID NO:3.
15. The CD20-binding protein of paragraph 10, wherein the protein comprises a light chain domain having an amino acid sequence of SEQ ID NO:4, SEQ ID NO:5, SEQ ID NO:6, SEQ ID NO:7, SEQ ID NO:8, SEQ ID NO:10, SEQ ID NO:11, SEQ ID NO:12, SEQ ID NO:13, SEQ ID NO:14, SEQ ID NO:91, SEQ ID NO:92 or SEQ ID NO:93.
16. The CD20-binding protein of paragraph 10, wherein the protein comprises a heavy chain domain having an amino acid sequence of SEQ ID NO:9, SEQ ID NO:89 or SEQ ID NO:94.
17. The CD20-binding protein of paragraph 10, comprising an Fc domain having an amino acid sequence of SEQ ID NO:15, SEQ ID NO:16, SEQ ID NO:17 or SEQ ID NO:18.
18. The CD20-binding protein of any of paragraphs 1-17, wherein the protein is in the form of a monoclonal antibody.
19. The CD20-binding protein of paragraph 18, wherein the monoclonal antibody is a chimeric, human or humanized monoclonal antibody.
20. The CD20-binding protein of any of paragraphs 1-17, wherein the protein is in the form of a fusion protein.
21. The CD20-binding protein of any of paragraphs 1-17, wherein the protein is in the form of an Fab antibody fragment, single-chain Fv antibody fragment, or minibody.
22. The CD20-binding protein of any of paragraphs 1-19, wherein the protein is conjugated to a diagnostic agent or a therapeutic agent.
23. A composition comprising the CD20-binding protein of any of paragraphs 1-22.
24. The composition of paragraph 23, wherein the protein is present at a concentration of about 50 mg/ml to about 250 mg/ml.
25. The composition of paragraph 24, wherein the protein is present at a concentration of about 100 mg/ml to about 200 mg/ml.
26. The composition of paragraph 25, wherein the protein is present at a concentration of about 110 mg/ml to about 150 mg/ml.
27. The composition of paragraph 26, wherein the protein is present at a concentration of about about 120 mg/ml.
28. The composition of paragraph 26, wherein the protein is present at a concentration of about about 130 mg/ml.
29. The composition of any of paragraphs 23-28, wherein the composition further comprises at least one buffer, at least one stabilizer, and/or at least one surfactant.
30. The composition of any of paragraphs 23-29, wherein the composition is liquid.
31. The composition of paragraph 30, wherein the composition is formulated for subcutaneous injection.
32. The composition of any of paragraphs 23-31, wherein the composition is sterile.
33. The composition of any of paragraphs 23-32, wherein the composition further comprises histidine HCl, trehalose dehydrate, methionine and/or polysorbate.
34. The composition of paragraph 23, wherein the composition comprises about 110 to about 130 mg/ml CD20-binding protein, about 10 mM to about 30 mM histidine HCl, about 200 mM to about 220 mM trehalose dehydrate, about 5 mM to about 15 mM methionine, and/or about 0.04% to about 0.08% polysorbate 80.
35. A method of treating a condition in a subject in need thereof, comprising administering to the subject a therapeutically effective amount of the composition of any of paragraphs 23-34.
36. The method of paragraph 35, wherein the condition is cancer or an autoimmune condition.
37. The method of paragraph 36, wherein the cancer is selected from B cell lymphoma, B cell leukemia, non-Hodgkin's lymphoma, lymphocyte predominant Hodgkin's disease, chronic lymphocytic leukemia, and small lymphocytic lymphoma.
38. The method of paragraph 36, wherein the autoimmune disease is selected from: rheumatoid arthritis, juvenile rheumatoid arthritis, systemic lupus erythematosus, lupus nephritis, ulcerative colitis, Wegener's disease, inflammatory bowel disease, idiopathic thrombocytopenic purpura, thrombotic thrombocytopenic purpura, autoimmune thrombocytopenia, multiple sclerosis, psoriasis, IgA nephropathy, IgM polyneuropathies, myasthenia gravis, vasculitis, diabetes mellitus, Reynaud's syndrome, Sjorgen's syndrome and glomerulonephritis.
39. A method of inducing apoptosis in B cells, comprising contacting B cells with the CD20-binding protein of any of paragraphs 1-22, thereby inducing apoptosis in the B cells.
40. A nucleic acid encoding the CD20-binding protein of any of paragraphs 1-22.
41. A vector comprising the nucleic acid of paragraph 40.
42. An expression cassette comprising the nucleic acid of paragraph 40.
43. A host cell comprising the nucleic acid of paragraph 40, the vector of paragraph 41, or the expression cassette of paragraph 42.
44. A method of producing an antibody, comprising culturing the host cell of paragraph 43.
45. A kit comprising a container and the CD20-binding protein of any of paragraphs 1-22 or the composition of any of paragraphs 23-34 contained therein.
46. The kit of paragraph 45, wherein the container is glass or plastic.
47. The kit of paragraph 45 or 46, wherein the container is a vial or a syringe.
48. The kit of any of paragraphs 45-47 wherein the kit further comprises instructions for using the kit.
49. The kit of any of paragraphs 45-48, wherein the kit further comprises a package insert or label indicating that the protein or composition can be used to treat cancer or an autoimmune condition characterized by the overexpression of CD20.
50. The kit of any of paragraphs 45-49, wherein the composition is provided at a volume of less than about 2 ml or is about 2 ml.
51. The kit of paragraph 50, wherein the composition is provided at a volume of about 1.5 ml.
52. An EGFR-binding protein comprising:

a light chain domain having an amino acid sequence of SEQ ID NO:34 modified to include at least one amino acid substitution selected from a neutral polar amino acid at L₃, a positive polar amino acid at L₃, a neutral polar amino acid at V₉, a negative polar amino acid at V₉, a neutral polar amino acid at I₁₀, and a negative polar amino acid at L₁₅₄, and a positive polar amino acid at L₁₅₄; and/or

a heavy chain domain having an amino acid sequence of SEQ ID NO:35 modified to include at least one amino acid substitution selected from a positive polar amino acid at L₉₉, a positive polar amino acid at L₁₁₄, a neutral polar amino acid at L₁₇₆, and a positive polar amino acid at L₁₇₆; and/or

an Fc domain having an amino acid sequence of SEQ ID NO:36 modified to include at least one amino acid substitution selected from: a positive polar amino acid at position L₁₉, a positive polar amino acid at position I₃₇, a positive polar amino acid at position V₆₆, and a positive polar amino acid at position L₉₃.

53. The EGFR-binding protein of paragraph 52, wherein the protein comprises a light chain domain having an amino acid sequence of SEQ ID NO:34 modified to include at least two, at least three, or at least four amino acid substitutions selected from a neutral polar amino acid at L₃, a positive polar amino acid at L₃, a neutral polar amino acid at V₉, a negative polar amino acid at V₉, a neutral polar amino acid at I₁₀, and a negative polar amino acid at L₁₅₄, and a positive polar amino acid at L₁₅₄.
54. The EGFR-binding protein of paragraph 52 or 53, wherein the neutral polar amino acid at L₃, V₉ and/or I₁₀ of SEQ ID NO:34 and/or at L₁₇₆ of SEQ ID NO:35 is asparagine (N), glutamine (Q), histidine (H), serine (S), threonine (T) or tyrosine (Y).
55. The EGFR-binding protein of any of paragraphs 52-54, wherein the negative polar amino acid at V₉ and/or L₁₅₄ of SEQ ID NO:34 is aspartic acid (D) or glutamic acid (E).
56. The EGFR-binding protein of any of paragraphs 52-55, wherein the positive polar amino acid at L₃ and/or L₁₅₄ of SEQ ID NO:34, L₉₉, L₁₁₄ and/or L₁₇₆ of SEQ ID NO:35, and/or L₁₉, I₃₇, V₆₆ and/or L₉₃ of SEQ ID NO: 36 is arginine (R) or lysine (K).
57. The EGFR-binding protein of any of paragraphs 52-56, wherein the protein comprises a light chain domain having an amino acid sequence of SEQ ID NO:34, a heavy chain domain having an amino acid sequence of SEQ ID NO:35, a human IgG Fc domain, or an Fc domain having an amino acid sequence of SEQ ID NO:36.
58. The EGFR-binding protein of paragraph 52, wherein the protein comprises a light chain domain having an amino acid sequence of SEQ ID NO:51, SEQ ID NO:52, SEQ ID NO:53, SEQ ID NO:54, SEQ ID NO:56, SEQ ID NO:57, SEQ ID NO:58, SEQ ID NO:59 or SEQ ID NO:60.
59. The EGFR-binding protein of paragraph 52, wherein the protein comprises a heavy chain domain having an amino acid sequence of SEQ ID NO:55, SEQ ID NO:95 or SEQ ID NO:96.
60. The EGFR-binding protein of paragraph 52, wherein the protein comprises an Fc chain domain having an amino acid sequence of SEQ ID NO:61, SEQ ID NO:62, SEQ ID NO:63 or SEQ ID NO:64.
61. An EGFR-binding protein comprising:

a light chain domain having an amino acid sequence of SEQ ID NO:34 modified to include at least one amino acid substitution selected from L₃Q, L₃K, V₉S, V₉D, I₁₀S, L₁₅₄K and L₁₅₄D; and/or

a heavy chain domain having an amino acid sequence of SEQ ID NO:35 modified to include at least one amino acid substitution selected from L₉₉K, L₁₁₄K, L₁₇₆K and L₁₇₆5; and/or

an Fc domain having an amino acid sequence of SEQ ID NO:36 modified to include at least one amino acid substitution selected from L₁₉K, I₃₇K, V₆₆K and L₉₃K.

62. The EGFR-binding protein of paragraph 61, wherein the protein comprises a light chain domain having an amino acid sequence of SEQ ID NO:34 modified to include at least two, at least three, or at least four amino acid substitutions selected from L₃Q, L₃K, V₉S, V₉D, I₁₀S, L₁₅₄K and L₁₅₄D.
63. The EGFR-binding protein of paragraph 61, wherein the protein comprises a heavy chain domain having an amino acid sequence of SEQ ID NO:35 modified to include at least two or at least three amino acid substitutions selected from L₉₉K, L₁₁₄K, L₁₇₆K and L₁₇₆S.
64. The EGFR-binding protein of paragraph 62, wherein the amino acid sequence of SEQ ID NO:34 is modified to include amino acid substitutions

(a) V₉S and I₁₀S,

(b) L₃Q and V₉S,

(c) L₃Q, V₉S and I₁₀S,

(d) L₃Q, V₉S and L₁₅₄D, or

(e) L₃Q, V₉S, I₁₀S and L₁₅₄D.

65. The EGFR-binding protein of any of paragraphs 61-64, wherein the protein comprises a light chain domain having an amino acid sequence of SEQ ID NO:34, a heavy chain domain having an amino acid sequence of SEQ ID NO:35, a human IgG Fc domain, or an Fc domain having an amino acid sequence of SEQ ID NO:36.
66. The EGFR-binding protein of paragraph 61, wherein the protein comprises a light chain domain having an amino acid sequence of SEQ ID NO:37, SEQ ID NO:38, SEQ ID NO:39, SEQ ID NO:40, SEQ ID NO:42, SEQ ID NO:43, SEQ ID NO:44, SEQ ID NO:45, SEQ ID NO:46, SEQ ID NO:97, SEQ ID NO:98 or SEQ ID NO:99.
67. The EGFR-binding protein of paragraph 61, wherein the protein comprises a heavy chain domain having an amino acid sequence of SEQ ID NO:41, SEQ ID NO:100, SEQ ID NO:101 or SEQ ID NO:102.
68. The EGFR-binding protein of paragraph 61, comprising an Fc domain domain having an amino acid sequence of SEQ ID NO:47, SEQ ID NO:48, SEQ ID NO:49 or SEQ ID NO:50.
69. The EGFR-binding protein of any of paragraphs 61-68, wherein the protein is in the form of a monoclonal antibody.
70. The EGFR-binding protein of paragraph 69, wherein the monoclonal antibody is a chimeric, human or humanized monoclonal antibody.
71. The EGFR-binding protein of any of paragraphs 61-68, wherein the protein is in the form of a fusion protein.
72. The EGFR-binding protein of any of paragraphs 61-68, wherein the protein is in the form of an Fab antibody fragment, single-chain Fv antibody fragment, or minibody.
73. The EGFR-binding protein of any of paragraphs 61-72, wherein the protein is conjugated to a diagnostic agent or a therapeutic agent.
74. A composition comprising the EGFR-binding protein of any of paragraphs 61-73.
75. The composition of paragraph 74, wherein the protein is present at a concentration of about 50 mg/ml to about 250 mg/ml.
76. The composition of paragraph 75, wherein the protein is present at a concentration of about 100 mg/ml to about 200 mg/ml.
77. The composition of paragraph 76 wherein the protein is present at a concentration of about 110 mg/ml to about 150 mg/ml.
78. The composition of paragraph 77, wherein the protein is present at a concentration of about about 120 mg/ml.
79. The composition of paragraph 77, wherein the protein is present at a concentration of about about 130 mg/ml.
80. The composition of any of paragraphs 75-79, wherein the composition further comprises at least one buffer, at least one stabilizer, and/or at least one surfactant.
81. The composition of any of paragraphs 74-80, wherein the composition is liquid.
82. The composition of paragraph 81, wherein the composition is formulated for subcutaneous injection.
83. The composition of any of paragraphs 74-82, wherein the composition is sterile.
84. The composition of any of paragraphs 74-83, wherein the composition further comprises histidine HCl, trehalose dehydrate, methionine and/or polysorbate.
85. The composition of paragraph 74, wherein the composition comprises about 110 to about 130 mg/ml EGFR-binding protein, about 10 mM to about 30 mM histidine HCl, about 200 mM to about 220 mM trehalose dehydrate, about 5 mM to about 15 mM methionine, and/or about 0.04% to about 0.08% polysorbate 80.
86. A method of treating a condition in a subject in need thereof, comprising administering to the subject a therapeutically effective amount of the composition of any of paragraphs 74-85.
87. The method of paragraph 86, wherein the condition is cancer.
88. The method of paragraph 87, wherein the cancer is selected from a brain tumor, a tumor of the urogenital tract, a tumor of the lymphatic system, a stomach tumor, a laryngeal tumor, monocytic leukemia, lung adenocarcinoma, small-cell lung carcinoma, pancreatic cancer, glioblastoma and breast carcinoma.
89. A nucleic acid encoding the EGFR-binding protein of any of paragraphs 52-73.
90. A vector comprising the nucleic acid of paragraph 89.
91. An expression cassette comprising the nucleic acid of paragraph 89.
92. A host cell comprising the nucleic acid of paragraph 89, the vector of paragraph 90, or the expression cassette of paragraph 91.
93. A method of producing an antibody, comprising culturing the host cell of paragraph 92.
94. A kit comprising a container and the EGFR-binding protein of any of paragraphs 52-73 or the composition of any of paragraphs 74-85 contained therein.
95. The kit of paragraph 94, wherein the container is glass or plastic. 96. The kit of paragraph 94 or 95, wherein the container is a vial or a syringe. 97. The kit of any of paragraphs 94-96, wherein the kit further comprises instructions for using the kit.
98. The kit of any of paragraphs 94-97, wherein the kit further comprises a package insert or label indicating that the protein or composition can be used to treat cancer or an autoimmune condition characterized by the overexpression of EGFR.
99. The kit of any of paragraphs 94-98, wherein the composition is provided at a volume of less than about 2 ml or is about to 2 ml.
100. The kit of paragraph 99, wherein the composition is provided at a volume of about 1.5 ml.

### EXAMPLES

### Example 1

Changes in aggregation tendency were assessed for select CD20-binding proteins of the invention, each having one or more amino acid substitution(s) (also referred to as modifications). Individual amino acid modifications are designated M1-M6, listed in Table III. The gene sequences (heavy chain and light chain sequences) used to express rituximab were obtained from U.S. Pat. No. 5,736,137, incorporated herein by reference. The rituximab heavy chain nucleotide sequence is provided herein as SEQ ID NO:73 and the light chain nucleotide sequence is provided herein as SEQ ID NO:67. The genes were synthesized by GENSCRIPT® and each subcloned in the GWIZ expression vector, mutated via PCR mutagenesis (primers synthesized by IDT), and transfected into HEK293 cells. The proteins were purified from the expression medium by capturing onto Protein A resin, with the elution further purified by cation exchange. The proteins were then formulated in 20 mM histidine HCl (pH 6.5) and concentrated to 20 mg/ml. To evaluate aggregation, solutions of each protein were incubated at an elevated temperature and analyzed via size exclusion HPLC. Binding to the targeted CD20 antigen was evaluated via an immunofluorescence assay where the proteins were incubated with Raji cells (B-Cells) and analyzed via flow cytometry after a fluorescently labeled secondary antibody was bound to the bound proteins.

TABLE III

| CD20-Binding | | | | |
|---|---|---|---|---|
| Protein | | Amino Acid | | Mutation/ |
| Modification | Chain | Position | Residue | Modification |
| M1 (V₃Q) | Light chain | 3 | Val | Gln |
| | (SEQ ID NO: 1) | | | |
| M2 (A₉S) | Light chain | 9 | Ala | Ser |
| | (SEQ ID NO: 1) | | | |
| M3 (I₁₀S) | Light chain | 10 | Ile | Ser |
| | (SEQ ID NO: 1) | | | |
| M4 (V₅₉S) | Light chain | 59 | Val | Ser |
| | (SEQ ID NO: 1) | | | |
| M5 (L₁₅₃K) | Light chain | 153 | Leu | Lys |
| | (SEQ ID NO: 1) | | | |
| M6 (L₁₇₈K) | Fab heavy chain | 178 | Leu | Lys |
| | (SEQ ID NO: 2) | | | |

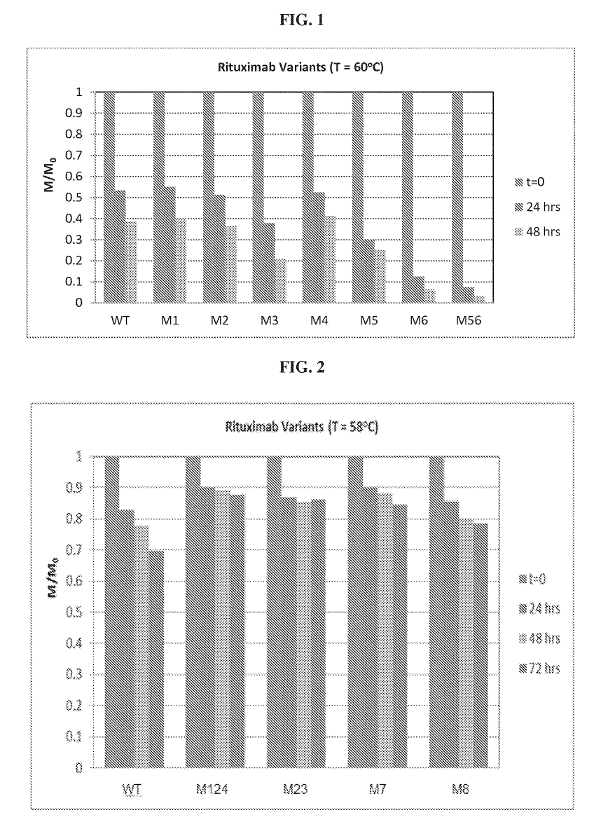

The effect of the amino acid modifications on protein aggregation are shown in FIG. 1. Several of the modifications had a neutral effect on aggregation (M1, M2 and M4), while others (M3, M5 and M6) had a destabilizing effect (increased aggregation).

### Example 2

Aggregation Assays.

Changes in aggregation tendency were assessed for several other CD20-binding proteins, each having one or more amino acid substitution(s). The additional proteins (M7-M9) are shown in Table IV. The protein with the M9 modification is located in the CDR of Rituximab.

TABLE IV

| CD20-Binding | | | | |
|---|---|---|---|---|
| Protein | | Amino Acid | | Mutation/ |
| Modification | Chain | Position | Residue | Modification |
| M1 (V₃Q) | Light chain | 3 | Val | Gln |
| | (SEQ ID NO: 1) | | | |
| M2 (A₉S) | Light chain | 9 | Ala | Ser |
| | (SEQ ID NO: 1) | | | |
| M3 (I₁₀S) | Light chain | 10 | Ile | Ser |
| | (SEQ ID NO: 1) | | | |
| M4 (V₅₉S) | Light chain | 59 | Val | Ser |
| | (SEQ ID NO: 1) | | | |
| M7 (L₁₅₃D) | Light chain | 153 | Leu | Asp |
| | (SEQ ID NO: 1) | | | |
| M8 (L₁₇₈S) | Fab heavy chain | 178 | Leu | Ser |
| | (SEQ ID NO: 2) | | | |
| M9 (Y₁₀₁S) | Fab heavy chain | 101 | Tyr | Ser |
| | (SEQ ID NO: 2) | | | |

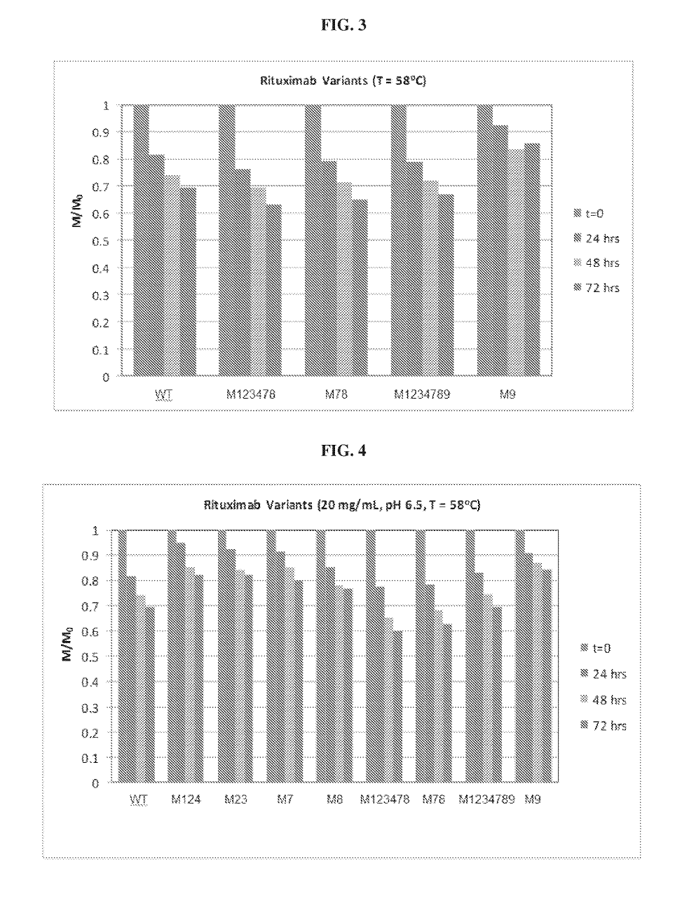

The following CD20-binding proteins with select combinations of amino acid modifications were generated and tested for aggregation tendency: M1/M2/M4; M2/M3; M1/M2/M3/M4/M7/M8; M7/M8; and M1/M2/M3/M4/M7/M8/M9. The amino acid substitution effects on protein aggregation are shown in FIGS. 2-5.

In this Example, the aggregation temperature was lowered to 58° C. because 60° C. appeared to be too harsh. Proteins with a combination of M1/M2/M4 modifications, proteins with a combination of M2/M3 modifications, and proteins with an M7 modification showed significant improvement in aggregation tendency (e.g., were more stable)—the aggregation rates were approximately 2-3 times slower than control proteins. Proteins with an M8 modification showed modest improvement. A photograph of the solutions after the incubation supported these aggregations results, showing that the solution containing wild type rituximab turned into a turbid gel, while the solution containing CD20-binding protein with an M8 modification turned only slightly cloudy, and solutions containing proteins with M1/M2/M4 modifications, M2/M3 modifications, or an M7 modification remained clear (data not shown).

CD20-binding proteins with a combination of M1/M2/M3/M4/M7/M8 modifications, M7/M8 modifications, and M1/M2/M3/M4/M7/M8/M9 modifications were destabilizing (e.g., increased protein aggregation) relative to wild type rituximab. A repeat aggregation experiment was conducted (see FIG. 4), which reproduced these results. A photograph of the solutions after the incubation supported these aggregation results, showing that the solution containing proteins with M1/M2/M3/M4/M7/M8 modifications and the solution containing proteins with M7/M8 modifications were the only solutions to become turbid during the experiment (data not shown).

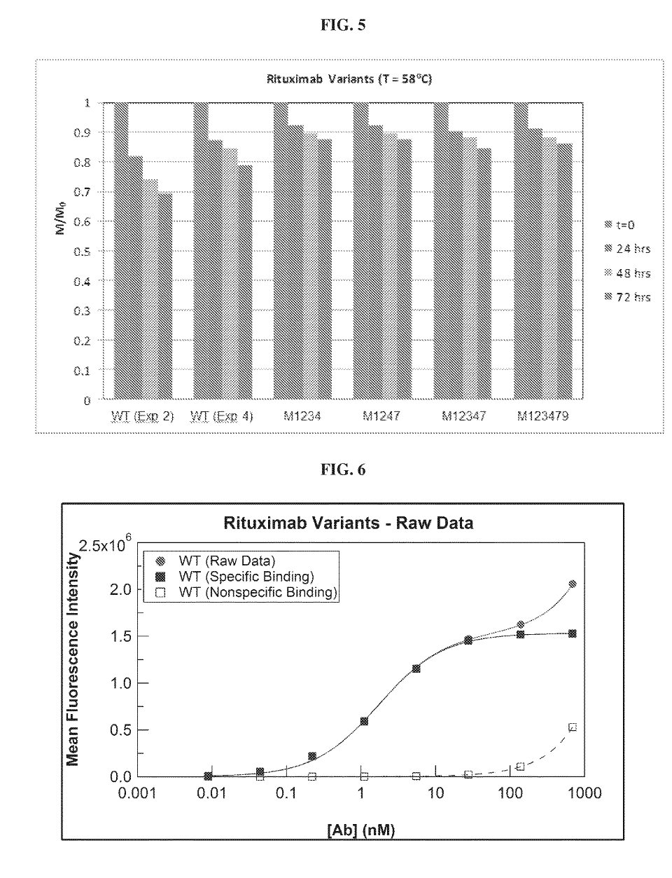

Those CD20-binding proteins that specifically excluded the M8 modification showed enhanced protein stability (see FIG. 5). This finding may be a result of batch-to-batch variation. FIG. 5 is a graph showing a comparison of two different batches of wild type rituximab (Exp 2 and Exp 4). Rituximab from the second batch (Exp 4) is more stable than rituximab from the first batch (Exp 2), though rituximab from the second batch is not as stable as the CD20-binding proteins shown in FIG. 5. Based on these results, there appears to be a limit to the extent to which improved stability can be achieved. There is little difference in the aggregation tendency among proteins with M2/M3 modifications, proteins with M1/M2/M4 modifications, proteins with M1/M2/M3/M4 modifications, proteins with M1/M2/M4/M7 modifications, proteins with M1/M2/M3/M4/M7 modifications, and proteins with M1/M2/M3/M4/M7/M8 modifications.

For a more direct comparison, rate constant values (fitted to a 2^{nd} order rate law) were computed for all of the CD20-binding proteins of the invention. Aggregation was slowed by up to a factor of 2.8 (M1/M2/M3/M4 & M1/M2/M4/M7) (Table V). Each of the CD20-binding proteins that contain an M6 mutation enhanced aggregation (e.g., decreased stability).

TABLE V

| Variant | k ml/(mg*hr) | k_{WT}/k |
|---|---|---|
| WT (Exp 2) | 3.18E−04 | 1.00 |
| WT (Exp 4) | 2.01E−04 | 1.58 |
| M7 | 1.41E−04 | 2.25 |
| M8 | 2.32E−04 | 1.37 |
| M9 | 1.48E−04 | 2.15 |
| M2/3 | 1.52E−04 | 2.09 |
| M7/8 | 4.14E−04 | 0.77 |
| M1/2/4 | 1.19E−04 | 2.67 |
| M1/2/3/4 | 1.14E−04 | 2.79 |
| M1/2/4/7 | 1.13E−04 | 2.80 |
| M1/2/3/4/7 | 1.40E−04 | 2.27 |
| M1/2/3/4/5/8 | 4.62E−04 | 0.69 |
| M123479 | 1.28E−04 | 2.49 |
| M1234789 | 3.94E−04 | 0.81 |

The results presented above show that the CD20-binding proteins presented herein reduce the rate of aggregation. There appears to be some discrepancy in the exact extent to which the amino acid modifications or the combination of modifications reduce aggregation. It is possible that there are differences in the glycosylation pattern from batch-to-batch that will influence aggregation but it is more likely that differences are the result of nucleation seeds (e.g., small aggregates, misfolded mAb, mAb fragments, and/or trace amounts of host cell protein or protein A) that differ in number from batch-to-batch, but are too few in number to be detected by SEC-HPLC or SDS-PAGE. If this is the case, the amount of pre-existing nucleation seeds may affect the initial loss of monomer.

Target Antigen Binding Assays.

Binding affinity of an antibody to its target antigen can be an indicator of antibody efficacy. An indirect immunofluorescence cell-based binding assay was conducted to determine the dissociation constant (KD) between select CD20-binding proteins of the invention and the targeted antigen (CD20 protein on the surface of B-Cells). FIG. 6 shows raw and analyzed binding curves for wild type rituximab. Wild type rituximab at varying concentrations was incubated with Raji cells (a Burkitt's lymphoma cell line). A fluorescently labeled secondary mAb (Alexafluor 488 goat anti-human IgG) was added, which bound to the bound rituximab, and the fluorescence intensity was determined via flow cytometry. The raw data (FIG. 6) indicated that there is both specific (single site saturation) and nonspecific (non-saturating) binding, with the latter becoming significant at high concentrations. Because of this, the raw data was fitted to the following equation:

$${f}_{Total} = \frac{{f}_{max} [ Ab ]}{[ Ab ] + {K}_{D}} + NS \cdot [ Ab ] ,$$

where the total fluorescence intensity observed (f_{Total}) is the combination of a non-cooperative hyperbolic binding curve (specific binding) and a linear binding curve (nonspecific binding). In the above equation, fₘₐₓ is the maximum amount of fluorescence intensity associated with all antigen binding bound to the mAb, [Ab] is the concentration of the antibody, and NS is the slope of the nonspecific binding curve. The analyzed curves presented in FIG. 6 are:

f_{nonspecific}=NS·[Ab]

f_{Specific}=f_{Total}−f_{Nonspecific}

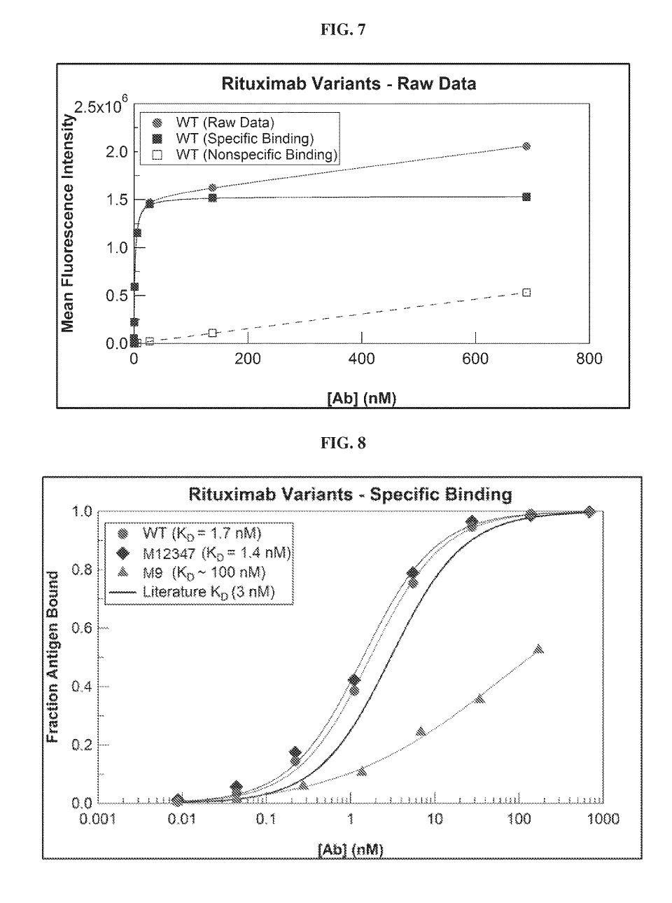

For clarity, rather than a log scale for mAb concentration (x-axis), FIG. 7 shows mAb concentration on a linear scale. It shows how the raw data exhibits nonsaturating behavior at higher concentrations that increase linearly. For variant binding, FIG. 8 shows the fraction antigen bound (fluorescence of specific binding relative to the computed fₘₐₓ) for the rituximab wild type, proteins with M1/M2/M3/M4/M7 modifications, and proteins with the M8 modification in addition to a binding curve computed from a KD value found in the literature. This plot shows information required in a minimal fashion to assess the effect of the amino acid modifications on antigen binding. Binding of wild type rituximab was compared to select CD20-binding proteins with all of the stabilizing mutations outside of the CDR (i.e., proteins with M1/M2/M3/M4/M7 modifications) and to proteins with a CDR mutation (M9). Wild type rituximab expressed in the lab and proteins with M1/M2/M3/M4/M7 modifications bind with the same affinity as the commercial product. Also, the CDR mutation eliminated the affinity for the target antigen.

SEQUENCES

SEQ ID NO: 1
QIVLSQSPAI LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 2
QVQLQQPGAE LVKPGASVKM SCKASGYTFT SYNMHWVKQT PGRGLEWIGA IYPGNGDTSY NQKFKGKATL
TADKSSSTAY MQLSSLTSED SAVYYCARST YYGGDWYFNV WGAGTTVTVS AASTKGPSVF PLAPSSKSTS
GGTAALGCLV KDYFPEPVTV SWNSGALTSG VHTFPAVLQS SGLYSLSSVV TVPSSSLGTQ TYICNVNHKP
SNTKVDKKVE PKSC
SEQ ID NO: 3
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVYT LPPSRDELTK
NQVSLTCLVK GFYPSDIAVE WESNGQPENN YKTTPPVLDS DGSFFLYSKL TVDKSRWQQG NVFSCSVMHE
ALHNHYTQKS LSLSPGK
SEQ ID NO: 4
QIQLSQSPAI LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 5
QIVLSQSPSI LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 6
QIVLSQSPAS LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 7
QIVLSQSPAI LSASPGEKVT MTCRASSSVS YIHWFQQKPG SSPKPWIYAT SNLASGVPSR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 8
QIVLSQSPAI LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NADQSGNSQE SVTEQDSKDS TYSLSSTLTL SKADYEKHKV YACEVTHQGL SSPVTKSFNR
GEC
SEQ ID NO: 9
QVQLQQPGAE LVKPGASVKM SCKASGYTFT SYNMHWVKQT PGRGLEWIGA IYPGNGDTSY NQKFKGKATL
TADKSSSTAY MQLSSLTSED SAVYYCARST YYGGDWYFNV WGAGTTVTVS AASTKGPSVF PLAPSSKSTS
GGTAALGCLV KDYFPEPVTV SWNSGALTSG VHTFPAVSQS SGLYSLSSVV TVPSSSLGTQ TYICNVNHKP
SNTKVDKKVE PKSC
SEQ ID NO: 10
QIVLSQSPSS LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 11
QIQLSQSPSI LSASPGEKVT MTCRASSSVS YIHWFQQKPG SSPKPWIYAT SNLASGVPSR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 12
QIQLSQSPSS LSASPGEKVT MTCRASSSVS YIHWFQQKPG SSPKPWIYAT SNLASGVPSR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 13
QIQLSQSPSI LSASPGEKVT MTCRASSSVS YIHWFQQKPG SSPKPWIYAT SNLASGVPSR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NADQSGNSQE SVTEQDSKDS TYSLSSTLTL SKADYEKHKV YACEVTHQGL SSPVTKSFNR
GEC
SEQ ID NO: 14
QIQLSQSPSS LSASPGEKVT MTCRASSSVS YIHWFQQKPG SSPKPWIYAT SNLASGVPSR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NADQSGNSQE SVTEQDSKDS TYSLSSTLTL SKADYEKHKV YACEVTHQGL SSPVTKSFNR
GEC
SEQ ID NO: 15
DKTHTCPPCP APELKGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVYT LPPSRDELTK
NQVSLTCLVK GFYPSDIAVE WESNGQPENN YKTTPPVLDS DGSFFLYSKL TVDKSRWQQG NVFSCSVMHE
ALHNHYTQKS LSLSPGK
SEQ ID NO: 16
DKTHTCPPCP APELLGGPSV FLFPPKPKDT LMKSRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVYT LPPSRDELTK
NQVSLTCLVK GFYPSDIAVE WESNGQPENN YKTTPPVLDS DGSFFLYSKL TVDKSRWQQG NVFSCSVMHE
ALHNHYTQKS LSLSPGK
SEQ ID NO: 17
DKTHTCPPCP APELLGGPSV FLFPPKPKDT LMISRTPEVT CVVVDVSHED PEVKFNWYVD GKEVHNAKTK
PREEQYNSTY RVVSVLTVLH QDWLNGKEYK CKVSNKALPA PIEKTISKAK GQPREPQVYT LPPSRDELTK
NQVSLTCLVK GFYPSDIAVE WESNGQPENN YKTTPPVLDS DGSFFLYSKL TVDKSRWQQG NVFSCSVMHE
ALHNHYTQKS LSLSPGK
SEQ ID NO: 18
DKTHTCPPCP APELLGGPSV FLFPPKPKDT LMISRTPEVT CVVVDVSHED PEVKFNWYVD GVEVHNAKTK
PREEQYNSTY RVVSVLTVKH QDWLNGKEYK CKVSNKALPA PIEKTISKAK GQPREPQVYT LPPSRDELTK
NQVSLTCLVK GFYPSDIAVE WESNGQPENN YKTTPPVLDS DGSFFLYSKL TVDKSRWQQG NVFSCSVMHE
ALHNHYTQKS LSLSPGK
SEQ ID NO: 19
QIXLSQSPAI LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 20
QIVLSQSPXI LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 21
QIVLSQSPAX LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 22
QIVLSQSPAI LSASPGEKVT MTCRASSSVS YIHWFQQKPG SSPKPWIYAT SNLASGVPXR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 23
QIVLSQSPAI LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NAXQSGNSQE SVTEQDSKDS TYSLSSTLTL SKADYEKHKV YACEVTHQGL SSPVTKSFNR
GEC
SEQ ID NO: 24
QVQLQQPGAE LVKPGASVKM SCKASGYTFT SYNMHWVKQT PGRGLEWIGA IYPGNGDTSY NQKFKGKATL
TADKSSSTAY MQLSSLTSED SAVYYCARST YYGGDWYFNV WGAGTTVTVS AASTKGPSVF PLAPSSKSTS
GGTAALGCLV KDYFPEPVTV SWNSGALTSG VHTFPAVXQS SGLYSLSSVV TVPSSSLGTQ TYICNVNHKP
SNTKVDKKVE PKSC
SEQ ID NO: 25
QIVLSQSPXX LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 26
QIXLSQSPXI LSASPGEKVT MTCRASSSVS YIHWFQQKPG SSPKPWIYAT SNLASGVPXR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 27
QIXLSQSPXX LSASPGEKVT MTCRASSSVS YIHWFQQKPG SSPKPWIYAT SNLASGVPXR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 28
QIXLSQSPXI LSASPGEKVT MTCRASSSVS YIHWFQQKPG SSPKPWIYAT SNLASGVPXR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NAXQSGNSQE SVTEQDSKDS TYSLSSTLTL SKADYEKHKV YACEVTHQGL SSPVTKSFNR
GEC
SEQ ID NO: 29
QIXLSQSPXX LSASPGEKVT MTCRASSSVS YIHWFQQKPG SSPKPWIYAT SNLASGVPXR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NAXQSGNSQE SVTEQDSKDS TYSLSSTLTL SKADYEKHKV YACEVTHQGL SSPVTKSFNR
GEC
SEQ ID NO: 30
DKTHTCPPCP APELXGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVYT LPPSRDELTK
NQVSLTCLVK GFYPSDIAVE WESNGQPENN YKTTPPVLDS DGSFFLYSKL TVDKSRWQQG NVFSCSVMHE
ALHNHYTQKS LSLSPGK
SEQ ID NO: 31
DKTHTCPPCP APELLGGPSV FLFPPKPKDT LMXSRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVYT LPPSRDELTK
NQVSLTCLVK GFYPSDIAVE WESNGQPENN YKTTPPVLDS DGSFFLYSKL TVDKSRWQQG NVFSCSVMHE
ALHNHYTQKS LSLSPGK
SEQ ID NO: 32
DKTHTCPPCP APELLGGPSV FLFPPKPKDT LMISRTPEVT CVVVDVSHED PEVKFNWYVD GXEVHNAKTK
PREEQYNSTY RVVSVLTVLH QDWLNGKEYK CKVSNKALPA PIEKTISKAK GQPREPQVYT LPPSRDELTK
NQVSLTCLVK GFYPSDIAVE WESNGQPENN YKTTPPVLDS DGSFFLYSKL TVDKSRWQQG NVFSCSVMHE
ALHNHYTQKS LSLSPGK
SEQ ID NO: 33
DKTHTCPPCP APELLGGPSV FLFPPKPKDT LMISRTPEVT CVVVDVSHED PEVKFNWYVD GVEVHNAKTK
PREEQYNSTY RVVSVLTVXH QDWLNGKEYK CKVSNKALPA PIEKTISKAK GQPREPQVYT LPPSRDELTK
NQVSLTCLVK GFYPSDIAVE WESNGQPENN YKTTPPVLDS DGSFFLYSKL TVDKSRWQQG NVFSCSVMHE
ALHNHYTQKS LSLSPGK
SEQ ID NO: 34
DILLTQSPVI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 35
QVQLKQSGPG LVQPSQSLSI TCTVSGFSLT NYGVHWVRQS PGKGLEWLGV IWSGGNTDYN TPFTSRLSIN
KDNSKSQVFF KMNSLQSNDT AIYYCARALT YYDYEFAYWG QGTLVTVSAA STKGPSVFPL APSSKSTSGG
TAALGCLVKD YFPEPVTVSW NSGALTSGVH TFPAVLQSSG LYSLSSVVTV PSSSLGTQTY ICNVNHKPSN
TKVDKRVEPK S
SEQ ID NO: 36
PKSCDKTHTC PPCPAPELLG GPSVFLFPPK PKDTLMISRT PEVTCVVVDV SHEDPEVKFN WYVDGVEVHN
AKTKPREEQY NSTYRVVSVL TVLHQDWLNG KEYKCKVSNK ALPAPIEKTI SKAKGQPREP QVYTLPPSRD
ELTKNQVSLT CLVKGFYPSD IAVEWESNGQ PENNYKTTPP VLDSDGSFFL YSKLTVDKSR WQQGNVFSCS
VMHEALHNHY TQKSLSLSPG K
SEQ ID NO: 37
DIQLTQSPVI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 38
DILLTQSPSI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 39
DILLTQSPVS LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 40
DILLTQSPVI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNADQSGNSQ ESVTEQDSKD STYSLSSTLT LSKADYEKHK VYACEVTHQG LSSPVTKSFN
RGEC
SEQ ID NO: 41
QVQLKQSGPG LVQPSQSLSI TCTVSGFSLT NYGVHWVRQS PGKGLEWLGV IWSGGNTDYN TPFTSRLSIN
KDNSKSQVFF KMNSLQSNDT AIYYCARALT YYDYEFAYWG QGTLVTVSAA STKGPSVFPL APSSKSTSGG
TAALGCLVKD YFPEPVTVSW NSGALTSGVH TFPAVSQSSG LYSLSSVVTV PSSSLGTQTY ICNVNHKPSN
TKVDKRVEPK S
SEQ ID NO: 42
DILLTQSPVI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 43
DILLTQSPVI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 44
DILLTQSPVI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 45
DILLTQSPVI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 46
DILLTQSPVI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 47
PKSCDKTHTC PPCPAPELKG GPSVFLFPPK PKDTLMISRT PEVTCVVVDV SHEDPEVKFN WYVDGVEVHN
AKTKPREEQY NSTYRVVSVL TVLHQDWLNG KEYKCKVSNK ALPAPIEKTI SKAKGQPREP QVYTLPPSRD
ELTKNQVSLT CLVKGFYPSD IAVEWESNGQ PENNYKTTPP VLDSDGSFFL YSKLTVDKSR WQQGNVFSCS
VMHEALHNHY TQKSLSLSPG K
SEQ ID NO: 48
PKSCDKTHTC PPCPAPELLG GPSVFLFPPK PKDTLMKSRT PEVTCVVVDV SHEDPEVKFN WYVDGVEVHN
AKTKPREEQY NSTYRVVSVL TVLHQDWLNG KEYKCKVSNK ALPAPIEKTI SKAKGQPREP QVYTLPPSRD
ELTKNQVSLT CLVKGFYPSD IAVEWESNGQ PENNYKTTPP VLDSDGSFFL YSKLTVDKSR WQQGNVFSCS
VMHEALHNHY TQKSLSLSPG K
SEQ ID NO: 49
PKSCDKTHTC PPCPAPELLG GPSVFLFPPK PKDTLMISRT PEVTCVVVDV SHEDPEVKFN WYVDGKEVHN
AKTKPREEQY NSTYRVVSVL TVLHQDWLNG KEYKCKVSNK ALPAPIEKTI SKAKGQPREP QVYTLPPSRD
ELTKNQVSLT CLVKGFYPSD IAVEWESNGQ PENNYKTTPP VLDSDGSFFL YSKLTVDKSR WQQGNVFSCS
VMHEALHNHY TQKSLSLSPG K
SEQ ID NO: 50
PKSCDKTHTC PPCPAPELLG GPSVFLFPPK PKDTLMISRT PEVTCVVVDV SHEDPEVKFN WYVDGVEVHN
AKTKPREEQY NSTYRVVSVL TVKHQDWLNG KEYKCKVSNK ALPAPIEKTI SKAKGQPREP QVYTLPPSRD
ELTKNQVSLT CLVKGFYPSD IAVEWESNGQ PENNYKTTPP VLDSDGSFFL YSKLTVDKSR WQQGNVFSCS
VMHEALHNHY TQKSLSLSPG K
SEQ ID NO: 51
DIXLTQSPVI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 52
DILLTQSPXI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 53
DILLTQSPVX LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 54
DILLTQSPVI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNAXQSGNSQ ESVTEQDSKD STYSLSSTLT LSKADYEKHK VYACEVTHQG LSSPVTKSFN
RGEC
SEQ ID NO: 55
QVQLKQSGPG LVQPSQSLSI TCTVSGFSLT NYGVHWVRQS PGKGLEWLGV IWSGGNTDYN TPFTSRLSIN
KDNSKSQVFF KMNSLQSNDT AIYYCARALT YYDYEFAYWG QGTLVTVSAA STKGPSVFPL APSSKSTSGG
TAALGCLVKD YFPEPVTVSW NSGALTSGVH TFPAVXQSSG LYSLSSVVTV PSSSLGTQTY ICNVNHKPSN
TKVDKRVEPK S
SEQ ID NO: 56
DILLTQSPXX LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 57
DIXLTQSPXI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 58
DIXLTQSPXX LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 59
DIXLTQSPXI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNAXQSGNSQ ESVTEQDSKD STYSLSSTLT LSKADYEKHK VYACEVTHQG LSSPVTKSFN
RGEC
SEQ ID NO: 60
DIXLTQSPXX LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNAXQSGNSQ ESVTEQDSKD STYSLSSTLT LSKADYEKHK VYACEVTHQG LSSPVTKSFN
RGEC
SEQ ID NO: 61
PKSCDKTHTC PPCPAPELXG GPSVFLFPPK PKDTLMISRT PEVTCVVVDV SHEDPEVKFN WYVDGVEVHN
AKTKPREEQY NSTYRVVSVL TVLHQDWLNG KEYKCKVSNK ALPAPIEKTI SKAKGQPREP QVYTLPPSRD
ELTKNQVSLT CLVKGFYPSD IAVEWESNGQ PENNYKTTPP VLDSDGSFFL YSKLTVDKSR WQQGNVFSCS
VMHEALHNHY TQKSLSLSPG K
SEQ ID NO: 62
PKSCDKTHTC PPCPAPELLG GPSVFLFPPK PKDTLMXSRT PEVTCVVVDV SHEDPEVKFN WYVDGVEVHN
AKTKPREEQY NSTYRVVSVL TVLHQDWLNG KEYKCKVSNK ALPAPIEKTI SKAKGQPREP QVYTLPPSRD
ELTKNQVSLT CLVKGFYPSD IAVEWESNGQ PENNYKTTPP VLDSDGSFFL YSKLTVDKSR WQQGNVFSCS
VMHEALHNHY TQKSLSLSPG K
SEQ ID NO: 63
PKSCDKTHTC PPCPAPELLG GPSVFLFPPK PKDTLMISRT PEVTCVVVDV SHEDPEVKFN WYVDGXEVHN
AKTKPREEQY NSTYRVVSVL TVLHQDWLNG KEYKCKVSNK ALPAPIEKTI SKAKGQPREP QVYTLPPSRD
ELTKNQVSLT CLVKGFYPSD IAVEWESNGQ PENNYKTTPP VLDSDGSFFL YSKLTVDKSR WQQGNVFSCS
VMHEALHNHY TQKSLSLSPG K
SEQ ID NO: 64
PKSCDKTHTC PPCPAPELLG GPSVFLFPPK PKDTLMISRT PEVTCVVVDV SHEDPEVKFN WYVDGVEVHN
AKTKPREEQY NSTYRVVSVL TVXHQDWLNG KEYKCKVSNK ALPAPIEKTI SKAKGQPREP QVYTLPPSRD
ELTKNQVSLT CLVKGFYPSD IAVEWESNGQ PENNYKTTPP VLDSDGSFFL YSKLTVDKSR WQQGNVFSCS
VMHEALHNHY TQKSLSLSPG K
SEQ ID NO: 65
MTTPRNSVNG TFPAEPMKGP IAMQSGPKPL FRRMSSLVGP TQSFFMRESK TLGAVQIMNG LFHIALGGLL
MIPAGIYAPI CVTVWYPLWG GIMYIISGSL LAATEKNSRK CLVKGKMIMN SLSLFAAISG MILSIMDILN
IKISHFLKME SLNFIRAHTP YINIYNCEPA NPSEKNSPST QYCYSIQSLF LGILSVMLIF AFFQELVIAG
IVENEWKRTC SRPKSNIVLL SAEEKKEQTI EIKEEVVGLT ETSSQPKNEE DIEIIPIQEE EEEETETNFP
EPPQDQESSP IENDSSP
SEQ ID NO: 66
MRPSGTAGAA LLALLAALCP ASRALEEKKV CQGTSNKLTQ LGTFEDHFLS LQRMFNNCEV VLGNLEITYV
QRNYDLSFLK TIQEVAGYVL IALNTVERIP LENLQIIRGN MYYENSYALA VLSNYDANKT GLKELPMRNL
QEILHGAVRF SNNPALCNVE SIQWRDIVSS DFLSNMSMDF QNHLGSCQKC DPSCPNGSCW GAGEENCQKL
TKIICAQQCS GRCRGKSPSD CCHNQCAAGC TGPRESDCLV CRKFRDEATC KDTCPPLMLY NPTTYQMDVN
PEGKYSFGAT CVKKCPRNYV VTDHGSCVRA CGADSYEMEE DGVRKCKKCE GPCRKVCNGI GIGEFKDSLS
INATNIKHFK NCTSISGDLH ILPVAFRGDS FTHTPPLDPQ ELDILKTVKE ITGFLLIQAW PENRTDLHAF
ENLEIIRGRT KQHGQFSLAV VSLNITSLGL RSLKEISDGD VIISGNKNLC YANTINWKKL FGTSGQKTKI
ISNRGENSCK ATGQVCHALC SPEGCWGPEP RDCVSCRNVS RGRECVDKCN LLEGEPREFV ENSECIQCHP
ECLPQAMNIT CTGRGPDNCI QCAHYIDGPH CVKTCPAGVM GENNTLVWKY ADAGHVCHLC HPNCTYGCTG
PGLEGCPTNG PKIPSIATGM VGALLLLLVV ALGIGLFMRR RHIVRKRTLR RLLQERELVE PLTPSGEAPN
QALLRILKET EFKKIKVLGS GAFGTVYKGL WIPEGEKVKI PVAIKELREA TSPKANKEIL DEAYVMASVD
NPHVCRLLGI CLTSTVQLIT QLMPFGCLLD YVREHKDNIG SQYLLNWCVQ IAKGMNYLED RRLVHRDLAA
RNVLVKTPQH VKITDFGLAK LLGAEEKEYH AEGGKVPIKW MALESILHRI YTHQSDVWSY GVTVWELMTF
GSKPYDGIPA SEISSILEKG ERLPQPPICT IDVYMIMVKC WMIDADSRPK FRELIIEFSK MARDPQRYLV
IQGDERMHLP SPTDSNFYRA LMDEEDMDDV VDADEYLIPQ QGFFSSPSTS RTPLLSSLSA TSNNSTVACI
DRNGLQSCPI KEDSFLQRYS SDPTGALTED SIDDTFLPVP EYINQSVPKR PAGSVQNPVY HNQPLNPAPS
RDPHYQDPHS TAVGNPEYLN TVQPTCVNST FDSPAHWAQK GSHQISLDNP DYQQDFFPKE AKPNGIFKGS
TAENAEYLRV APQSSEFIGA
SEQ ID NO: 67
GTC GAC ATG GAT TTT CAG GTG CAG ATT ATC AGC TTC CTG CTA ATC AGT GCT TCA GTC
ATA ATG TCC AGA GGA CAA ATT GTT CTC TCC CAG TCT CCA GCA ATC CTG TCT GCA TCT
CCA GGG GAG AAG GTC ACA ATG ACT TGC AGG GCC AGC TCA AGT GTA AGT TAC ATC CAC
TGG TTC CAG CAG AAG CCA GGA TCC TCC CCC AAA CCC TGG ATT TAT GCC ACA TCC AAC
CTG GCT TCT GGA GTC CCT GTT CGC TTC AGT GGC AGT GGG TCT GGG ACT TCT TAC TCT
CTC ACA ATC AGC AGA GTG GAG GCT GAA GAT GCT GCC ACT TAT TAC TGC CAG CAG TGG
ACT AGT AAC CCA CCC ACG TTC GGA GGG GGG ACC AAG CTG GAA ATC AAA CGT ACG GTG
GCT GCA CCA TCT GTC TTC ATC TTC CCG CCA TCT GAT GAG CAG TTG AAA TCT GGA ACT
GCC TCT GTT GTG TGC CTG CTG AAT AAC TTC TAT CCC AGA GAG GCC AAA GTA CAG TGG
AAG GTG GAT AAC GCC CTC CAA TCG GGT AAC TCC CAG GAG AGT GTC ACA GAG CAG GAC
AGC AAG GAC AGC ACC TAC AGC CTC AGC AGC ACC CTG ACG CTG AGC AAA GCA GAC TAC
GAG AAA CAC AAA GTC TAC GCC TGC GAA GTC ACC CAT CAG GGC CTG AGC TCG CCC GTC
ACA AAG AGC TTC AAC AGG GGA GAG TGT TGA GCG GCC GC
SEQ ID NO: 68
CC AGA GGA CAA ATT CAA CTC TCC CAG TCT CC
SEQ ID NO: 69
CC CAG TCT CCA TCA ATC CTG TCT GC
SEQ ID NO: 70
AG TCT CCA GCA AGC CTG TCT GCA TC
SEQ ID NO: 71
CT GGA GTC CCT AGT CGC TTC AGT GG
SEQ ID NO: 72
GTG GAT AAC GCC GAC CAA TCG GGT AAC
SEQ ID NO: 73
GTC GAC ATG GGT TGG AGC CTC ATC TTG CTC TTC CTT GTC GCT GTT GCT ACG CGT GTC
CTG TCC CAG GTA CAA CTG CAG CAG CCT GGG GCT GAG CTG GTG AAG CCT GGG GCC TCA
GTG AAG ATG TCC TGC AAG GCT TCT GGC TAC ACA TTT ACC AGT TAC AAT ATG CAC TGG
GTA AAA CAG ACA CCT GGT CGG GGC CTG GAA TGG ATT GGA GCT ATT TAT CCC GGA AAT
GGT GAT ACT TCC TAC AAT CAG AAG TTC AAA GGC AAG GCC ACA TTG ACT GCA GAC AAA
TCC TCC AGC ACA GCC TAC ATG CAG CTC AGC AGC CTG ACA TCT GAG GAC TCT GCG GTC
TAT TAC TGT GCA AGA TCG ACT TAC TAC GGC GGT GAC TGG TAC TTC AAT GTC TGG GGC
GCA GGG ACC ACG GTC ACC GTC TCT GCA GCT AGC ACC AAG GGC CCA TCG GTC TTC CCC
CTG GCA CCC TCC TCC AAG AGC ACC TCT GGG GGC ACA GCG GCC CTG GGC TGC CTG GTC
AAG GAC TAC TTC CCC GAA CCG GTG ACG GTG TCG TGG AAC TCA GGC GCC CTG ACC AGC
GGC GTG CAC ACC TTC CCG GCT GTC CTA CAG TCC TCA GGA CTC TAC TCC CTC AGC AGC
GTG GTG ACC GTG CCC TCC AGC AGC TTG GGC ACC CAG ACC TAC ATC TGC AAC GTG AAT
CAC AAG CCC AGC AAC ACC AAG GTG GAC AAG AAA GCA GAG CCC AAA TCT TGT GAC AAA
ACT CAC ACA TGC CCA CCG TGC CCA GCA CCT GAA CTC CTG GGG GGA CCG TCA GTC TTC
CTC TTC CCC CCA AAA CCC AAG GAC ACC CTC ATG ATC TCC CGG ACC CCT GAG GTC ACA
TGC GTG GTG GTG GAC GTG AGC CAC GAA GAC CCT GAG GTC AAG TTC AAC TGG TAC GTG
GAC GGC GTG GAG GTG CAT AAT GCC AAG ACA AAG CCG CGG GAG GAG CAG TAC AAC AGC
ACG TAC CGT GTG GTC AGC GTC CTC ACC GTC CTG CAC CAG GAC TGG CTG AAT GGC AAG
GAG TAC AAG TGC AAG GTC TCC AAC AAA GCC CTC CCA GCC CCC ATC GAG AAA ACC ATC
TCC AAA GCC AAA GGG CAG CCC CGA GAA CCA CAG GTG TAC ACC CTG CCC CCA TCC CGG
GAT GAG CTG ACC AAG AAC CAG GTC AGC CTG ACC TGC CTG GTC AAA GGC TTC TAT CCC
AGC GAC ATC GCC GTG GAG TGG GAG AGC AAT GGG CAG CCG GAG AAC AAC TAC AAG ACC
ACG CCT CCC GTG CTG GAC TCC GAC GGC TCC TTC TTC CTC TAC AGC AAG CTC ACC GTG
GAC AAG AGC AGG TGG CAG CAG GGG AAC GTC TTC TCA TGC TCC GTG ATG CAT GAG GCT
CTG CAC AAC CAC TAC ACG CAG AAG AGC CTC TCC CTG TCT CCG GGT AAA TGA GCG GCC
GC
SEQ ID NO: 74
C TTC CCG GCT GTC TCA CAG TCC TCA GG
SEQ ID NO: 75
GCA AGA TCG ACT TCC TAC GGC GGT G
SEQ ID NO: 77
GTC GAC ATG GAT TTT CAG GTG CAG ATT ATC AGC TTC CTG CTA ATC AGT GCT TCA GTC
ATA ATG TCC AGA GGA GAC ATC CTG TTG ACT CAG AGC CCT GTG ATC CTG TCC GTG TCC
CCT GGC GAG AGG GTC TCC TTC TCT TGT CGA GCG AGC CAG TCC ATC GGC ACC AAC ATC
CAC TGG TAT CAG CAG AGG ACC AAC GGG TCC CCC AGG CTC CTT ATT AAG TAT GCT AGC
GAG AGC ATC AGC GGC ATC CCC TCC AGG TTC TCC GGC AGC GGT AGC GGA ACC GAC TTC
ACC CTT TCT ATC AAC TCC GTG GAG AGC GAG GAC ATC GCG GAC TAT TAC TGC CAG CAG
AAT AAC AAC TGG CCA ACC ACC TTC GGC GCA GGC ACT AAG CTG GAG CTG AAG CGC ACC
GTG GCG GCT CCC AGC GTG TTC ATC TTC CCC CCG AGC GAC GAG CAG CTG AAA AGC GGT
ACT GCA AGC GTC GTT TGC CTC CTG AAC AAC TTC TAC CCC AGG GAG GCC AAG GTG CAG
TGG AAA GTG GAC AAC GCC CTG CAG AGC GGG AAC AGC CAA GAG TCA GTG ACC GAG CAG
GAC TCC AAG GAC AGC ACC TAC AGC CTC AGC TCA ACG CTG ACC CTG AGC AAG GCC GAT
TAC GAG AAG CAC AAG GTG TAC GCC TGT GAG GTG ACC CAT CAG GGC CTG TCT TCT CCC
GTC ACC AAA AGC TTC AAC AGA GGC GAG TGC TAA GCG GCC GC
SEQ ID NO: 77
GGA GAC ATC CAG TTG ACT CAG AGC
SEQ ID NO: 78
GCT CTG AGT CAA CTG GAT GTC TCC
SEQ ID NO: 79
ACT CAG AGC CCT TCG ATC CTG TCC GTG TC
SEQ ID NO: 80
GAC ACG GAC AGG ATC GAA GGG CTC TGA GT
SEQ ID NO: 81
G TTG ACT CAG AGC CCT GTG AGC CTG TCC GTG TC
SEQ ID NO: 82
GAC ACG GAC AGG CTC ACA GGG CTC TGA GTC AAC
SEQ ID NO: 83
G TTG ACT CAG AGC CCT TCG AGC CTGT CCG TG
SEQ ID NO: 84
CAC GGA CAG GCT CGA AGG GCT CTG AGT CAA C
SEQ ID NO: 85
TG GAC AAC GCC GAT CAG AGC GGG AAC AG
SEQ ID NO: 86
CTG TTC CCG CTC TGA TCG GCG TTG TCC A
SEQ ID NO: 87
GTC GAC ATG GGT TGG AGC CTC ATC TTG CTC TTC CTT GTC GCT GTT GCT ACG CGT GTC
CTG TCC CAG GTG CAG CTG AAG CAG AGC GGC CCG GGG CTC GTC CAG CCC TCG CAG AGC
CTG AGC ATC ACC TGC ACG GTG AGC GGC TTC AGC CTG ACC AAC TAC GGG GTG CAC TGG
GTC CGG CAG TCG CCC GGC AAG GGG CTC GAG TGG CTG GGC GTG ATC TGG AGC GGC GGG
AAC ACC GAC TAC AAC ACC CCC TTC ACG AGC CGC CTG AGC ATC AAC AAG GAC AAC AGC
AAG TCG CAG GTG TTC TTC AAG ATG AAC AGC CTC CAG AGC AAC GAC ACC GCC ATC TAC
TAC TGC GCG CGG GCC CTG ACC TAC TAC GAC TAC GAG TTC GCC TAC TGG GGC CAG GGG
ACC CTG GTC ACG GTG AGC GCC GCG AGC ACC AAG GGC CCG AGC GTG TTC CCC CTC GCC
CCC TCG AGC AAG AGC ACC AGC GGC GGG ACC GCC GCC CTG GGC TGC CTG GTC AAG GAC
TAC TTC CCC GAG CCG GTG ACG GTG AGC TGG AAC TCG GGG GCC CTC ACC AGC GGC GTC
CAC ACC TTC CCC GCG GTG CTG CAG AGC AGC GGG CTG TAC AGC CTC AGC TCG GTG GTC
ACC GTG CCC AGC AGC AGC CTG GGC ACG CAG ACC TAC ATC TGC AAC GTG AAC CAC AAG
CCC AGC AAC ACC AAG GTC GAC AAG CGC GTG GAG CCG AAG TCG CCC AAG AGC TGC GAC
AAG ACC CAC ACG TGC CCG CCC TGC CCC GCC CCC GAG CTG CTC GGC GGG CCC AGC GTG
TTC CTG TTC CCG CCC AAG CCC AAG GAC ACC CTG ATG ATC AGC CGG ACC CCC GAG GTC
ACC TGC GTG GTG GTC GAC GTG AGC CAC GAG GAT CCG GAG GTG AAG TTC AAC TGG TAC
GTC GAC GGC GTG GAG GTG CAC AAC GCC AAG ACG AAG CCC CGC GAG GAG CAG TAC AAC
AGC ACC TAC CGG GTC GTG TCG GTG CTC ACC GTC CTG CAC CAG GAC TGG CTG AAC GGG
AAG GAG TAC AAG TGC AAG GTG AGC AAC AAG GCC CTC CCC GCG CCC ATC GAG AAG ACC
ATC AGC AAG GCC AAG GGC CAG CCG CGC GAG CCC CAG GTG TAC ACG CTG CCC CCC AGC
CGG GAC GAG CTG ACC AAG AAC CAG GTC AGC CTC ACC TGC CTG GTG AAG GGG TTC TAC
CCG TCG GAC ATC GCC GTG GAG TGG GAG AGC AAC GGC CAG CCC GAG AAC AAC TAC AAG
ACC ACG CCC CCG GTC CTG GAC AGC GAC GGC AGC TTC TTC CTC TAC AGC AAG CTG ACC
GTG GAC AAG AGC CGC TGG CAG CAG GGG AAC GTG TTC TCG TGC AGC GTC ATG CAC GAG
GCC CTG CAC AAC CAC TAC ACC CAG AAG AGC CTC AGC CTG AGC CCC GGC AAG TGA TAA
GCG GCC GC
SEQ ID NO: 88
QVQLQQPGAE LVKPGASVKM SCKASGYTFT SYNMHWVKQT PGRGLEWIGA IYPGNGDTSY NQKFKGKATL
TADKSSSTAY MQLSSLTSED SAVYYCARST XYGGDWYFNV WGAGTTVTVS AASTKGPSVF PLAPSSKSTS
GGTAALGCLV KDYFPEPVTV SWNSGALTSG VHTFPAVLQS SGLYSLSSVV TVPSSSLGTQ TYICNVNHKP
SNTKVDKKVE PKSC
SEQ ID NO: 89
QVQLQQPGAE LVKPGASVKM SCKASGYTFT SYNMHWVKQT PGRGLEWIGA IYPGNGDTSY NQKFKGKATL
TADKSSSTAY MQLSSLTSED SAVYYCARST HYGGDWYFNV WGAGTTVTVS AASTKGPSVF PLAPSSKSTS
GGTAALGCLV KDYFPEPVTV SWNSGALTSG VHTFPAVLQS SGLYSLSSVV TVPSSSLGTQ TYICNVNHKP
SNTKVDKKVE PKSC
SEQ ID NO: 90
QIVLSQSPAI LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKK YACEVTHQGL SSPVTKSFNR
GEC
SEQ ID NO: 91
QIVLSQSPAK LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 92
QIVLSQSPAI LSASPGEKVT MTCRASSSVS YIHWFQQKPG SSPKPWIYAT SNLASGVPDR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 93
QIVLSQSPAI LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTKA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 94
QVQLQQPGAE LVKPGASVKM SCKASGYTFT SYNMHWVKQT PGRGLEWIGA IYPGNGDTSY NQKFKGKATL
TADKSSSTAY MQLSSLTSED SAVYYCARST YYGGDWYFNV WGAGTTVTVS AASTKGPSVF PLAPSSKSTS
GGTAALGCLV KDYFPEPVTV SWNSGALTSG VHTFPAVKQS SGLYSLSSVV TVPSSSLGTQ TYICNVNHKP
SNTKVDKKVE PKSC
SEQ ID NO: 95
QVQLKQSGPG LVQPSQSLSI TCTVSGFSLT NYGVHWVRQS PGKGLEWLGV IWSGGNTDYN TPFTSRLSIN
KDNSKSQVFF KMNSLQSNDT AIYYCARXT YYDYEFAYWG QGTLVTVSAA STKGPSVFPL APSSKSTSGG
TAALGCLVKD YFPEPVTVSW NSGALTSGVH TFPAVLQSSG LYSLSSVVTV PSSSLGTQTY ICNVNHKPSN
TKVDKRVEPK S
SEQ ID NO: 96
QVQLKQSGPG LVQPSQSLSI TCTVSGFSLT NYGVHWVRQS PGKGLEWLGV IWSGGNTDYN TPFTSRLSIN
KDNSKSQVFF KMNSLQSNDT AIYYCARALT YYDYEFAYWG QGTXVTVSAA STKGPSVFPL APSSKSTSGG
TAALGCLVKD YFPEPVTVSW NSGALTSGVH TFPAVLQSSG LYSLSSVVTV PSSSLGTQTY ICNVNHKPSN
TKVDKRVEPK S
SEQ ID NO: 97
DIKLTQSPVI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 98
DILLTQSPDI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 99
DILLTQSPVI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNAKQSGNSQ ESVTEQDSKD STYSLSSTLT LSKADYEKHK VYACEVTHQG LSSPVTKSFN
RGEC
SEQ ID NO: 100
QVQLKQSGPG LVQPSQSLSI TCTVSGFSLT NYGVHWVRQS PGKGLEWLGV IWSGGNTDYN TPFTSRLSIN
KDNSKSQVFF KMNSLQSNDT AIYYCARAKT YYDYEFAYWG QGTLVTVSAA STKGPSVFPL APSSKSTSGG
TAALGCLVKD YFPEPVTVSW NSGALTSGVH TFPAVLQSSG LYSLSSVVTV PSSSLGTQTY ICNVNHKPSN
TKVDKRVEPK S
SEQ ID NO: 101
QVQLKQSGPG LVQPSQSLSI TCTVSGFSLT NYGVHWVRQS PGKGLEWLGV IWSGGNTDYN TPFTSRLSIN
KDNSKSQVFF KMNSLQSNDT AIYYCARALT YYDYEFAYWG QGTKVTVSAA STKGPSVFPL APSSKSTSGG
TAALGCLVKD YFPEPVTVSW NSGALTSGVH TFPAVLQSSG LYSLSSVVTV PSSSLGTQTY ICNVNHKPSN
TKVDKRVEPK S
SEQ ID NO: 102
QVQLKQSGPG LVQPSQSLSI TCTVSGFSLT NYGVHWVRQS PGKGLEWLGV IWSGGNTDYN TPFTSRLSIN
KDNSKSQVFF KMNSLQSNDT AIYYCARALT YYDYEFAYWG QGTLVTVSAA STKGPSVFPL APSSKSTSGG
TAALGCLVKD YFPEPVTVSW NSGALTSGVH TFPAVKQSSG LYSLSSVVTV PSSSLGTQTY ICNVNHKPSN
TKVDKRVEPK S

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A CD20-binding protein comprising:
a light chain domain having an amino acid sequence of SEQ ID NO:1 modified to include at least one amino acid substitution selected from the group consisting of: a neutral polar amino acid at V₃, a neutral polar amino acid at 9, a neutral polar amino acid at I₁₀, a neutral polar amino acid at V₅₉ and a negatively charged polar amino acid at L₁₅₃; and/or
a heavy chain domain having an amino acid sequence of SEQ ID NO:2 modified to include an amino acid substitution selected from the group consisting of a neutral polar amino acid at Y₁₀₁ and a neutral polar amino acid at L₁₇₈; and/or
an Fc domain having an amino acid sequence of SEQ ID NO:3 modified to include at least one amino acid substitution selected from the group consisting of: a positive polar amino acid at position L₁₅, a positive polar amino acid at position I₃₃, a positive polar amino acid at position V₆₂, and a positive polar amino acid at position L₈₉.

2. The CD20-binding protein of claim 1, wherein the protein comprises a light chain domain having an amino acid sequence of SEQ ID NO:1 modified to include at least two, at least three, at least four, or at least five amino acid substitutions selected from the group consisting of: a neutral polar amino acid at V₃, a neutral polar amino acid at 9, a neutral polar amino acid at I₁₀, a neutral polar amino acid at V₅₉, and a negatively charged polar amino acid at L₁₅₃.

3. The CD20-binding protein of claim 1 or 2, wherein the neutral polar amino acid at V₃, 9, I₁₀ and V₅₉ of SEQ ID NO:1 and at Y₁₀₁ and L₁₇₈ of SEQ ID NO:2 is selected from the group consisting of: asparagine (N), cysteine (C), glutamine (Q), histidine (H), serine (S), threonine (T) or tyrosine (Y).

4. The CD20-binding protein of any of claims 1-3, wherein the negatively charged polar amino acid at L₁₅₃ of SEQ ID NO:1 is aspartic acid (D) or glutamic acid (E).

5. The CD20-binding protein of any of claims 1-4, wherein the positive polar amino acid at L₁₅, I₃₃, V₆₂ and L₈₉ of SEQ ID NO:3 is arginine (R) or lysine (K).

6. The CD20-binding protein of any of any of claims 1-5, wherein the protein comprises a light chain domain having an amino acid sequence of SEQ ID NO:1, a heavy chain domain having an amino acid sequence of SEQ ID NO:2, a human IgG Fc domain, or an Fc domain having an amino acid sequence of SEQ ID NO:3.

7. The CD20-binding protein of claim 1, wherein the protein comprises a light chain domain having an amino acid sequence of SEQ ID NO:19, SEQ ID NO:20, SEQ ID NO:21, SEQ ID NO:22, SEQ ID NO:23, SEQ ID NO:25, SEQ ID NO:26, SEQ ID NO:27, SEQ ID NO:28, or SEQ ID NO:29.

8. The CD20-binding protein of claim 1, wherein the protein comprises a heavy chain domain having an amino acid sequence of SEQ ID NO:24 or SEQ ID NO:88.

9. The CD20-binding protein of claim 1, wherein the protein comprises an Fc chain domain having an amino acid sequence of SEQ ID NO:30, SEQ ID NO:31, SEQ ID NO:32, or SEQ ID NO:33.

10. A CD20-binding protein comprising:
a light chain domain having an amino acid sequence of SEQ ID NO:1 modified to include at least one amino acid substitution selected from the group consisting of: V₃Q, 9S, I₁₀S, V₅₉5 and L₁₅₃D; and/or
a heavy chain domain having an amino acid sequence of SEQ ID NO:2 modified to include an amino acid substitution selected from the group consisting of a neutral polar amino acid at Y₁₀₁H and a neutral polar amino acid at L₁₇₈S; and/or
an Fc domain having an amino acid sequence of SEQ ID NO:3 modified to include at least one amino acid substitution selected from the group consisting of: L₁₅K, I₃₃K, V₆₂K and L₈₉K.

11. The CD20-binding protein of claim 10, wherein the protein comprises a light chain domain having an amino acid sequence of SEQ ID NO:1 modified to include at least two, at least three, at least four, or at least five amino acid substitutions selected from the group consisting of: V₃Q, 9S, T₁₀S, V₅₉S and L₁₅₃D.

12. The CD20-binding protein of claim 11, wherein the amino acid sequence of SEQ ID NO:1 is modified to include amino acid substitutions
(f) 9S and I₁₀S,
(g) V₃Q, 9S and V₅₉S,
(h) V₃Q, 9S, I₁₀S and V₅₉S,
(i) V₃Q, 9S, V₅₉S and L₁₅₃D, or
(j) V₃Q, 9S, I₁₀S, V₅₉S and L₁₅₃D.

13. The CD20-binding protein of any of any of claims 10-12, wherein the protein comprises a light chain domain having an amino acid sequence of SEQ ID NO:1, a heavy chain domain having an amino acid sequence of SEQ ID NO:2, a human IgG Fc domain, or an Fc domain having an amino acid sequence of SEQ ID NO:3.

14. The CD20-binding protein of claim 10, wherein the protein comprises a light chain domain having an amino acid sequence of SEQ ID NO:4, SEQ ID NO:5, SEQ ID NO:6, SEQ ID NO:7, SEQ ID NO:8, SEQ ID NO:10, SEQ ID NO:11, SEQ ID NO:12, SEQ ID NO:13, or SEQ ID NO:14.

15. The CD20-binding protein of claim 10, wherein the protein comprises a heavy chain domain having an amino acid sequence of SEQ ID NO:9.

16. The CD20-binding protein of claim 10, comprising an Fc domain having an amino acid sequence of SEQ ID NO:15, SEQ ID NO:16, SEQ ID NO:17, or SEQ ID NO:18.

17. The CD20-binding protein of any of claims 1-16, wherein the protein is in the form of a monoclonal antibody

18. The CD20-binding protein of claim 17, wherein the monoclonal antibody is a chimeric, human or humanized monoclonal antibody.

19. The CD20-binding protein of any of claims 1-16, wherein the protein is in the form of a fusion protein.

20. The CD20-binding protein of any of claims 1-16, wherein the protein is in the form of an Fab antibody fragment, single-chain Fv antibody fragment, or minibody.

21. The CD20-binding protein of any of claims 1-20, wherein the protein is conjugated to a diagnostic agent or a therapeutic agent.

22. A composition comprising the CD20-binding protein of any of claims 1-21.

23. The composition of claim 22, wherein the protein is present at a concentration of about 50 mg/ml to about 250 mg/ml.

24. The composition of claim 23, wherein the protein is present at a concentration of about 100 mg/ml to about 200 mg/ml.

25. The composition of claim 24, wherein the protein is present at a concentration of about 110 mg/ml to about 150 mg/ml.

26. The composition of claim 25, wherein the protein is present at a concentration of about about 120 mg/ml.

27. The composition of claim 25, wherein the protein is present at a concentration of about about 130 mg/ml.

28. The composition of any of claims 22-27, wherein the composition further comprises at least one buffer, at least one stabilizer, and/or at least one surfactant.

29. The composition of any of claims 22-28, wherein the composition is liquid.

30. The composition of claim 29, wherein the composition is formulated for subcutaneous injection.

31. The composition of any of claims 22-30, wherein the composition is sterile.

32. The composition of any of claims 22-31, wherein the composition further comprises histidine HCl, trehalose dehydrate, methionine and/or polysorbate.

33. The composition of claim 22, wherein the composition comprises about 110 to about 130 mg/ml CD20-binding protein, about 10 mM to about 30 mM histidine HCl, about 200 mM to about 220 mM trehalose dehydrate, about 5 mM to about 15 mM methionine, and/or about 0.04% to about 0.08% polysorbate 80.

34. A method of treating a condition in a subject in need thereof, comprising administering to the subject a therapeutically effective amount of the composition of any of claims 22-33.

35. The method of claim 34, wherein the condition is cancer or an autoimmune condition.

36. The method of claim 35, wherein the cancer is selected from the group consisting of: B cell lymphoma, B cell leukemia, non-Hodgkin's lymphoma, lymphocyte predominant Hodgkin's disease, chronic lymphocytic leukemia, and small lymphocytic lymphoma.

37. The method of claim 35, wherein the autoimmune disease is selected from the group consisting of: rheumatoid arthritis, juvenile rheumatoid arthritis, systemic lupus erythematosus, lupus nephritis, ulcerative colitis, Wegener's disease, inflammatory bowel disease, idiopathic thrombocytopenic purpura, thrombotic thrombocytopenic purpura, autoimmune thrombocytopenia, multiple sclerosis, psoriasis, IgA nephropathy, IgM polyneuropathies, myasthenia gravis, vasculitis, diabetes mellitus, Reynaud's syndrome, Sjorgen's syndrome and glomerulonephritis.

38. A method of inducing apoptosis in B cells, comprising contacting B cells with the CD20-binding protein of any of claims 1-21, thereby inducing apoptosis in the B cells.

39. A nucleic acid encoding the CD20-binding protein of any of claims 1-21.

40. A vector comprising the nucleic acid of claim 39.

41. An expression cassette comprising the nucleic acid of claim 39.

42. A host cell comprising the nucleic acid of claim 39, the vector of claim 40, or the expression cassette of claim 41.

43. A method of producing an antibody, comprising culturing the host cell of claim 42.

44. A kit comprising a container and the CD20-binding protein of any of claims 1-21 or the composition of any of claims 22-23 contained therein.

45. The kit of claim 44, wherein the container is glass or plastic.

46. The kit of claim 44 or 45, wherein the container is a vial or a syringe.

47. The kit of any of claims 44-46, wherein the kit further comprises instructions for using the kit.

48. The kit of any of claims 44-47, wherein the kit further comprises a package insert or label indicating that the protein or composition can be used to treat cancer or an autoimmune condition characterized by the overexpression of CD20.

49. The kit of any of claims 44-48, wherein the composition is provided at a volume of less than about 2 ml or is about 2 ml.

50. The kit of claim 49, wherein the composition is provided at a volume of about 1.5 ml.

51. An EGFR-binding protein comprising:
a light chain domain having an amino acid sequence of SEQ ID NO:34 modified to include at least one amino acid substitution selected from the group consisting of: a neutral polar amino acid at L₃, a neutral polar amino acid at V₉, a neutral polar amino acid at I₁₀, and a negatively charged polar amino acid at L₁₅₄; and/or
a heavy chain domain having an amino acid sequence of SEQ ID NO:35 modified to include an amino acid substitution of a neutral polar amino acid at L₁₇₆; and/or
an Fc domain having an amino acid sequence of SEQ ID NO:36 modified to include at least one amino acid substitution selected from the group consisting of: a positive polar amino acid at position L₁₉, a positive polar amino acid at position I₃₇, a positive polar amino acid at position V₆₆, and a positive polar amino acid at position L₉₃.

52. The EGFR-binding protein of claim 51, wherein the protein comprises a light chain domain having an amino acid sequence of SEQ ID NO:34 modified to include at least two, at least three, or at least four amino acid substitutions selected from the group consisting of: a neutral polar amino acid at L₃, a neutral polar amino acid at V₉, a neutral polar amino acid at I₁₀, and a negatively charged polar amino acid at L₁₅₃.

53. The EGFR-binding protein of claim 51 or 52, wherein the neutral polar amino acid at L₃, V₉ and I₁₀ of SEQ ID NO:34 and at L₁₇₆ of SEQ ID NO:35 is selected from the group consisting of: asparagine (N), cysteine (C), glutamine (Q), histidine (H), serine (S), threonine (T) or tyrosine (Y).

54. The EGFR-binding protein of any of claims 51-53, wherein the negatively charged polar amino acid at L₁₅₄ of SEQ ID NO:34 is aspartic acid (D) or glutamic acid (E).

55. The EGFR-binding protein of any of claims 51-54, wherein the positive polar amino acid at L₁₉, I₃₇, V₆₆ or L₉₃ of SEQ ID NO: 36 is arginine (R) or lysine (K).

56. The EGFR-binding protein of any of any of claims 51-55, wherein the protein comprises a light chain domain having an amino acid sequence of SEQ ID NO:34, a heavy chain domain having an amino acid sequence of SEQ ID NO:35, a human IgG Fc domain, or an Fc domain having an amino acid sequence of SEQ ID NO:36.

57. The EGFR-binding protein of claim 51, wherein the protein comprises a light chain domain having an amino acid sequence of SEQ ID NO:51, SEQ ID NO:52, SEQ ID NO:53, SEQ ID NO:54, SEQ ID NO:56, SEQ ID NO:57, SEQ ID NO:58, SEQ ID NO:59, or SEQ ID NO:60.

58. The EGFR-binding protein of claim 51, wherein the protein comprises a heavy chain domain having an amino acid sequence of SEQ ID NO:55.

59. The EGFR-binding protein of claim 51, wherein the protein comprises an Fc chain domain having an amino acid sequence of SEQ ID NO:61, SEQ ID NO:62, SEQ ID NO:63 or SEQ ID NO:64.

60. An EGFR-binding protein comprising:
a light chain domain having an amino acid sequence of SEQ ID NO:34 modified to include at least one amino acid substitution selected from the group consisting of: L₃Q, V₉S, I₁₀S and L₁₅₄D; and/or
a heavy chain domain having an amino acid sequence of SEQ ID NO:35 modified to include an amino acid substitution of L₁₇₆S; and/or
an Fc domain having an amino acid sequence of SEQ ID NO:36 modified to include at least one amino acid substitution selected from the group consisting of: L₁₉K, I₃₇K, V₆₆K and L₉₃K.

61. The EGFR-binding protein of claim 60, wherein the protein comprises a light chain domain having an amino acid sequence of SEQ ID NO:34 modified to include at least two, at least three, or at least four amino acid substitutions selected from the group consisting of: L₃Q, V₉S, I₁₀S and I₁₅₄D.

62. The EGFR-binding protein of claim 61, wherein the amino acid sequence of SEQ ID NO:34 is modified to include amino acid substitutions
(f) V₉S and I₁₀S,
(g) L₃Q and V₉S,
(h) L₃Q, V₉S and I₁₀S,
(i) L₃Q, V₉S and L₁₅₄D, or
(j) L₃Q, V₉5, 110 S and L₁₅₄D.

63. The EGFR-binding protein of any of any of claims 60-62, wherein the protein comprises a light chain domain having an amino acid sequence of SEQ ID NO:34, a heavy chain domain having an amino acid sequence of SEQ ID NO:35, a human IgG Fc domain, or an Fc domain having an amino acid sequence of SEQ ID NO:36.

64. The EGFR-binding protein of claim 60, wherein the protein comprises a light chain domain having an amino acid sequence of SEQ ID NO:37, SEQ ID NO:38, SEQ ID NO:39, SEQ ID NO:40, SEQ ID NO:42, SEQ ID NO:43, SEQ ID NO:44, SEQ ID NO:45, or SEQ ID NO:46.

65. The EGFR-binding protein of claim 60, wherein the protein comprises a heavy chain domain having an amino acid sequence of SEQ ID NO:41.

66. The EGFR-binding protein of claim 60, comprising an Fc domain domain having an amino acid sequence of SEQ ID NO:47, SEQ ID NO:48, SEQ ID NO:49 or SEQ ID NO:50.

67. The EGFR-binding protein of any of claims 51-66, wherein the protein is in the form of a monoclonal antibody.

68. The EGFR-binding protein of claim 67, wherein the monoclonal antibody is a chimeric, human or humanized monoclonal antibody.

69. The EGFR-binding protein of any of claims 51-66, wherein the protein is in the form of a fusion protein.

70. The EGFR-binding protein of any of claims 51-66, wherein the protein is in the form of an Fab antibody fragment, single-chain Fv antibody fragment, or minibody.

71. The EGFR-binding protein of any of claims 51-70, wherein the protein is conjugated to a diagnostic agent or a therapeutic agent.

72. A composition comprising the EGFR-binding protein of any of claims 51-71.

73. The composition of claim 72, wherein the protein is present at a concentration of about 50 mg/ml to about 250 mg/ml.

74. The composition of claim 73, wherein the protein is present at a concentration of about 100 mg/ml to about 200 mg/ml.

75. The composition of claim 74, wherein the protein is present at a concentration of about 110 mg/ml to about 150 mg/ml.

76. The composition of claim 75, wherein the protein is present at a concentration of about about 120 mg/ml.

77. The composition of claim 75, wherein the protein is present at a concentration of about about 130 mg/ml.

78. The composition of any of claims 72-77, wherein the composition further comprises at least one buffer, at least one stabilizer, and/or at least one surfactant.

79. The composition of any of claims 72-78, wherein the composition is liquid.

80. The composition of claim 79, wherein the composition is formulated for subcutaneous injection.

81. The composition of any of claims 72-80, wherein the composition is sterile.

82. The composition of any of claims 72-81, wherein the composition further comprises histidine HCl, trehalose dehydrate, methionine and/or polysorbate.

83. The composition of claim 72, wherein the composition comprises about 110 to about 130 mg/ml EGFR-binding protein, about 10 mM to about 30 mM histidine HCl, about 200 mM to about 220 mM trehalose dehydrate, about 5 mM to about 15 mM methionine, and/or about 0.04% to about 0.08% polysorbate 80.

84. A method of treating a condition in a subject in need thereof, comprising administering to the subject a therapeutically effective amount of the composition of any of claims 72-83.

85. The method of claim 84, wherein the condition is cancer.

86. The method of claim 85, wherein the cancer is selected from the group consisting of: a brain tumor, a tumor of the urogenital tract, a tumor of the lymphatic system, a stomach tumor, a laryngeal tumor, monocytic leukemia, lung adenocarcinoma, small-cell lung carcinoma, pancreatic cancer, glioblastoma, breast carcinoma.

87. A nucleic acid encoding the EGFR-binding protein of any of claims 72-83.

88. A vector comprising the nucleic acid of claim 87.

89. An expression cassette comprising the nucleic acid of claim 87.

90. A host cell comprising the nucleic acid of claim 87, the vector of claim 88, or the expression cassette of claim 89.

91. A method of producing an antibody, comprising culturing the host cell of claim 90.

92. A kit comprising a container and the EGFR-binding protein of any of claims 51-71 or the composition of any of claims 72-83 contained therein.

93. The kit of claim 92, wherein the container is glass or plastic.

94. The kit of claim 92 or 93, wherein the container is a vial or a syringe.

95. The kit of any of claims 92-94, wherein the kit further comprises instructions for using the kit.

96. The kit of any of claims 92-95, wherein the kit further comprises a package insert or label indicating that the protein or composition can be used to treat cancer or an autoimmune condition characterized by the overexpression of EGFR.

97. The kit of any of claims 92-96, wherein the composition is provided at a volume of less than about 2 ml or is about to 2 ml.
